(19) United States
(12) Patent Application Publication
(10) Pub. No.: US 2025/0099375 A9
(43) Pub. Date: Mar. 27, 2025

(54) Expandable multi-excipient dosage form

(71) Applicant: Aron H. Blaesi, Cambridge, MA (US)

(72) Inventors: Aron H. Blaesi, Cambridge, MA (US); Nannaji Saka, Cambridge, MA (US)

(73) Assignee: Aron H. Blaesi, Cambridge, MA (US)

(21) Appl. No.: 17/327,721

(22) Filed: May 23, 2021

Related U.S. Application Data
(63) Continuation of application No. PCT/US2021/022857, filed on Mar. 17, 2021.
(60) Provisional application No. 62/991,052, filed on Mar. 17, 2020.
(60) Provisional application No. 63/158,870, filed on Mar. 9, 2021.
(60) Provisional application No. 63/085,893, filed on Sep. 30, 2020.

(51) Int. Cl. A61K 9/00; A61K 9/70; A61K 47/32; A61K 47/38

(52) U.S. Cl. CPC A61K 9/0065; A61K 9/70; A61K 47/32; A61K 47/38

(57) ABSTRACT

Many drug therapies could be greatly improved by dosage forms that reside in the stomach for prolonged time and release the drug slowly. In this specification, therefore, an expandable, multi-excipient dosage form for prolonged release is disclosed. The dosage form generally comprises a three-dimensional structural framework of solid elements. The elements comprise at least a drug, at least a physiological fluid-absorptive excipient, and at least a strength-enhancing excipient. Upon ingestion, the three-dimensional structural framework expands in at least one dimension and forms an expanded semi-solid mass that can be retained in the stomach and release drug over prolonged time.

### CROSS-REFERENCE TO RELATED INVENTIONS

This application is a continuation of, and incorporates herein by reference in its entirety, the International Application No. PCT/US2021/022857 filed on Mar. 17, 2021 and titled “Expandable, multi-excipient structured dosage form for prolonged drug release”, which claims priority to and the benefit of the U.S. Provisional Application No. 62/991,052 filed on Mar. 17, 2020, the U.S. Provisional Application No. 63/085,893 filed on Sep. 30, 2020, and the U.S. Provisional Application No. 63/158,870 filed on Mar. 9, 2021. All foregoing applications are hereby incorporated by reference in their entirety.

This application is related to, and incorporates herein by reference in their entirety, the U.S. application Ser. No.15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, the U.S. application Ser. No. 16/860,911 filed on Apr. 28, 2020 and titled “Expandable structured dosage form for immediate drug delivery”, the U.S. application Ser. No. 16/916,208 filed on Jun. 30, 2020 and titled “Dosage form comprising structural framework of two-dimensional elements”, the International Application No. PCT/US19/19004 filed on Feb. 21, 2019 and titled “Expanding structured dosage form”, and the International Application No. PCT/US19/52030 filed on Sep. 19, 2019 and titled “Dosage form comprising structured solid-solution framework of sparingly-soluble drug and method for manufacture thereof”.

### BACKGROUND OF THE INVENTION

The prevalent oral-delivery dosage forms, the tablets and capsules, are porous solids of compacted drug and excipient particles. As shown schematically in FIG. 1a, the typical ingested dosage form may fragment into its particulate constituents in the stomach and release drug molecules. The drug particles and molecules may then traverse along the gastrointestinal tract, and the drug molecules may be absorbed by the blood stream. Drug that reaches the end of the gastrointestinal tract may be excreted.

By the traditional solid dosage forms, however, many kinds of drug cannot be optimally delivered. For example, drugs that are soluble at very low pH, but insoluble at higher pH, may be absorbed only in the upper gastrointestinal tract. The residence time in the upper part is generally short, which may limit the amount of drug absorbed and the bioavailability, and preclude prolonged drug delivery. Consequently, the efficacy, safety, and convenience of the drug therapy may be compromised.

Drug absorption could be extended by dosage forms that reside in the stomach for prolonged time and release drug slowly. Indeed, over the years several gastroretentive devices have been proposed. The most common are the floating and the expandable dosage forms.

The floating dosage forms are designed to float over the gastric contents in the upper stomach, thus preventing their passage into the small intestine. The concept, however, generally requires that the stomach is frequently filled with food and drink, and that the patient is in the upright posture. Because of these impractical requirements such dosage forms may not be preferred.

Expandable dosage forms should be smaller than the diameter of the esophagus (˜15 mm) to facilitate ingestion, FIG. 1b. But in the stomach they should expand to a size greater than the diameter of the pylorus (˜13-20 mm) to preclude passage into the small intestine. To date, however, a safe, ingestible dosage form that expands rapidly and releases drug slowly into the stomach is not available.

Accordingly, in the International Application No. PCT/US19/19004 the present inventors (Blaesi and Saka) have introduced fibrous dosage forms that expand rapidly due to fast water absorption by the thin fibers. The dosage form may then form a viscous gel from which drug molecules are released slowly.

In the prior disclosure, the non-limiting experimental dosage forms that expanded to twice their initial length in 15 minutes released 80% of the drug in about two hours. In some cases, however, the therapeutic benefits of the expandable, gastroretentive dosage forms may be even greater if the drug release time could be further prolonged.

In the present disclosure, therefore, new formulations and dosage form microstructures are presented to stabilize and strengthen the expanded dosage form without compromising its fast expansion. Concepts for controlling and extending the range of the drug release time from the stabilized, expanded dosage form are also disclosed.

### SUMMARY OF THE INVENTION

Generally, the dosage forms disclosed herein comprise a three-dimensional structural framework of solid elements. The elements comprise at least a drug, at least a physiological fluid-absorptive excipient, and at least a strength-enhancing excipient. Upon ingestion, the three-dimensional structural framework expands in at least one dimension and forms an expanded semi-solid mass that can be retained in the stomach and release drug over prolonged time.

More specifically, in one aspect, the invention herein comprises a fiber for pharmaceutical dosage form fabrication or construction comprising at least one active ingredient and at least two excipients forming the fiber; said at least two excipients comprising one or more fluid-absorptive polymeric constituents and one or more strength-enhancing polymeric constituents; wherein upon exposure to physiological fluid, said one or more strength-enhancing excipients form a fluid-permeable, semi-solid network mechanically supporting the fiber; and said one or more fluid-absorptive excipients transition to a viscous mass or a viscous solution expanding said fiber along at least one dimension with absorption of said physiological fluid.

In another aspect, the invention herein comprises a fiber for pharmaceutical dosage form fabrication comprising at least one active ingredient and at least two excipients forming the fiber; said at least two excipients comprising one or more fluid-absorptive polymeric constituents within which the solubility of a physiological fluid (e.g., gastric fluid) is greater than 600 mg/ml; said at least two excipients further comprising one or more strength-enhancing polymeric constituents; said one or more strength-enhancing polymeric constituents having an elastic modulus in the range between 0.2 MPa and 500 MPa and a strain at fracture greater than 0.2 after soaking with a physiological fluid (e.g., gastric fluid) under physiological conditions; wherein upon exposure to a physiological fluid, said one or more strength-enhancing excipients form a fluid-permeable, semi-solid network mechanically supporting the fiber; and said one or more fluid-absorptive excipients transition to a viscous mass or a viscous solution expanding said fiber along at least one dimension with absorption of said physiological fluid.

In some embodiments, the solubility of physiological fluid in the absorptive excipient is greater than 750 mg/ml.

In some embodiments, rate of penetration of physiological/body fluid into an absorptive excipient under physiological conditions is greater than the average thickness of the fiber, element, or elements divided by 3600 seconds.

In some embodiments, at least one absorptive excipient comprises hydroxypropyl methylcellulose.

In some embodiments, the molecular weight of said hydroxypropyl methylcellulose excipient is in the range between 30 kg/mol and 1000 kg/mol (e.g., between 50 kg/mol and 300 kg/mol).

In some embodiments, at least one absorptive excipient is selected from the group comprising hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, or vinylpyrrolidone-vinyl acetate copolymer.

In some embodiments, the molecular weight of at least one absorptive excipient is in the range of 30 kg/mol to 100,000 kg/mol (e.g., between 50 kg/mol and 100,000 kg/mol).

In some embodiments, the solubility of a relevant physiological fluid in at least a strength-enhancing excipient is no greater than 750 mg/ml (e.g., no greater than 600 mg/ml) under physiological conditions.

In some embodiments, at least a strength-enhancing excipient comprises an elastic modulus in the range of 0.3 MPa-150 MPa (e.g., 0.5 MPa-100 MPa) after soaking with a physiological fluid under physiological conditions.

In some embodiments, at least a strength-enhancing excipient comprises a tensile strength in the range of 0.05 MPa-200 MPa (e.g., 0.1 MPa-100 MPa) after soaking with a physiological fluid under physiological conditions.

In some embodiments, at least a strength-enhancing excipient comprises a strain at fracture greater than 0.3 (e.g., greater than 0.4, or greater than 0.5, or greater than 0.6) after soaking with a physiological fluid under physiological conditions.

In some embodiments, the volume or weight fraction of the one or more absorptive excipients in the fiber is in the range between 0.1 and 0.85 (e.g., between 0.15 and 0.8, or between 0.15 and 0.75).

In some embodiments, the volume or weight fraction of the one or more strength-enhancing excipients in the fiber is in the range between 0.15 and 0.9 (e.g. 0.2-0.9, 0.25-0.9, 0.3-0.9).

In some embodiments, at least one strength-enhancing excipient comprises an enteric polymer.

In some embodiments, at least one strength-enhancing excipient comprises an enteric polymer, said enteric polymer having a solubility at least 10 times greater in basic solution having a pH value greater than 7 than in acidic solution having a pH value no greater than 5.

In some embodiments, at least one strength-enhancing excipient comprises methacrylic acid-ethyl acrylate copolymer.

In some embodiments, at least one strength-enhancing excipient is selected from the group comprising hydroxypropyl methyl cellulose acetate succinate, polyvinyl acetate, ethyl acrylate polymers (e.g., polymers including ethyl acrylate), methacrylate polymers (e.g., polymers including methacrylate), ethyl acrylate-methylmethacrylate copolymers, Poly[Ethyl acrylate, methyl methacrylate, trimethylammonioethyl methacrylate chloride], Poly[Ethyl acrylate, methyl methacrylate, trimethylammonioethyl methacrylate chloride], and ethylcellulose.

In some embodiments, said at least two excipients form a solid solution through the thickness of the fiber.

In some embodiments, one or more phases comprising strength-enhancing excipient are substantially connected or substantially contiguous along the length of the fiber.

In some embodiments, said fiber comprises a plurality of segments having substantially the same weight fraction of physiological fluid-absorptive excipient distributed within the segments.

In some embodiments, said fiber comprises a plurality of segments having substantially the same weight fraction of strength-enhancing excipient distributed within the segments.

In some embodiments, upon exposure to a physiological fluid under physiological conditions, the diffusivity of absorptive polymeric excipient through said fiber is no greater than 10⁻¹² m²/s (e.g., no greater than 0.5×10⁻¹² m²/s, or no greater than 0.2×10⁻¹² m²/s).

In some embodiments, upon exposure to a physiological fluid under physiological conditions, the diffusivity of said physiological fluid through said fiber is greater than 0.2×10⁻¹² m²/s (e.g., greater than 0.5×10⁻¹² m²/s, or greater than 10⁻¹² m²/s).

In some embodiments, upon exposure to a physiological fluid, said fiber expands to a length between 1.3 and 4 times its length prior to exposure to said physiological fluid.

In some embodiments, upon exposure to a physiological fluid, said fiber expands in all dimensions.

In some embodiments, upon exposure to a physiological fluid, said fiber transitions to a semi-solid mass.

In some embodiments, upon exposure to a physiological fluid, said fiber transitions to a semi-solid mass, and wherein the one or more strength-enhancing excipients form a connected network through the semi-solid mass.

In some embodiments, said expanded fiber or semi-solid mass maintains its length between 1.3 and 4 times the initial length for prolonged time upon prolonged exposure to a physiological fluid.

In some embodiments, an expanded semi-solid mass comprises an elastic modulus in the range of 0.005 MPa-30 MPa (e.g., between 0.005 MPa-20 MPa, or 0.02 MPa-20 MPa).

In some embodiments, an expanded semi-solid mass comprises a tensile strength in the range between 0.002 MPa and 20 MPa (e.g., between 0.005 MPa and 15 MPa).

In another aspect, the invention herein comprises a pharmaceutical dosage form comprising a drug-containing solid comprising an outer surface and an internal three dimensional structural framework of one or more thin structural elements, said framework contiguous with and terminating at said outer surface; said elements having segments spaced apart from adjoining segments, thereby defining one or more free spaces in the drug-containing solid; said elements further comprising at least one active ingredient and at least two excipients; said at least two excipients comprising at least one physiological fluid-absorptive polymeric constituent and at least one strength-enhancing polymeric constituent; whereby upon immersion in a physiological fluid, said fluid percolates at least one free space and diffuses into one or more said elements, so that the framework expands in at least one dimension and transitions to a semi-solid mass; wherein said semi-solid mass releases the drug over prolonged time.

In some embodiments, upon exposure to a physiological fluid, said strength-enhancing excipient forms a fluid-permeable, semi-solid network to mechanically support said framework; and said fluid-absorptive excipient transitions to a semi-solid or viscous mass expanding said framework along at least one dimension with absorption of said physiological fluid.

In a further aspect, a pharmaceutical dosage form comprises a drug-containing solid comprising an outer surface and an internal three dimensional structural framework of one or more thin structural elements, said framework contiguous with and terminating at said outer surface; said elements having segments spaced apart from adjoining segments, thereby defining one or more interconnected free spaces through the drug-containing solid; said elements further comprising at least one active ingredient and at least two excipients; said at least two excipients comprising at least one physiological fluid-absorptive polymeric constituent and at least one strength-enhancing polymeric constituent; wherein upon exposure to a physiological fluid, said strength-enhancing excipient forms a fluid-permeable, semi-solid network mechanically supporting said framework; and said fluid-absorptive excipient transitions to a viscous mass or a viscous solution expanding said framework along at least one dimension with absorption of said physiological fluid.

In a further aspect a pharmaceutical dosage form herein comprises a drug-containing solid comprising an outer surface and an internal three dimensional structural framework of one or more thin structural elements, said framework contiguous with and terminating at said outer surface; said elements having segments spaced apart from adjoining segments, thereby defining one or more interconnected free spaces through the drug-containing solid; said elements further comprising at least one active ingredient and at least two excipients; said at least two excipients comprising one or more fluid-absorptive polymeric constituents within which the solubility of a physiological fluid (e.g., gastric fluid) is greater than 600 mg/ml; said at least two excipients further comprising one or more strength-enhancing polymeric constituents; said one or more strength-enhancing polymeric constituents having an elastic modulus in the range between 0.1 MPa and 500 MPa and a strain at fracture greater than 0.2 after soaking with a physiological fluid (e.g., gastric fluid) under physiological conditions; wherein upon exposure to a physiological fluid, said one or more strength-enhancing excipients form a fluid-permeable, semi-solid network mechanically supporting the fiber; and said one or more fluid-absorptive excipients transition to a viscous mass or a viscous solution expanding said fiber along at least one dimension with absorption of said physiological fluid.

In some embodiments, one or more phases comprising strength-enhancing excipient form a substantially continuous or connected structure along the lengths of one or more structural elements.

In some embodiments, one or more phases comprising strength-enhancing excipient form a substantially continuous or connected structure through the three dimensional structural framework.

In some embodiments, upon ingestion by a human or animal subject, physiological fluid percolates at least one free space and diffuses into one or more said elements, thereby expanding said framework in all dimensions and transitioning said framework to a semi-solid mass releasing said drug over time.

In some embodiments, upon exposure to a physiological fluid, said framework expands to a length between 1.3 and 4 times its length prior to exposure to said physiological fluid.

In some embodiments, upon prolonged exposure to a physiological fluid, said expanded framework or semi-solid mass maintains its length between 1.3 and 4 times the initial length for prolonged time.

In some embodiments, the semi-solid mass comprises a substantially continuous or connected network of one or more strength-enhancing excipients.

In some embodiments, the semi-solid mass comprises a substantially continuous or connected network of strength-enhancing excipient that extends over the length, width, and thickness of said semi-solid mass.

In some embodiments, one or more phases comprising strength-enhancing excipient extend along the lengths of the structural elements.

In some embodiments, the average thickness of the one or more structural elements is in the range of 1 μm to 1.5 mm.

In some embodiments, one or more interconnected free spaces form an open pore network that extends over a length at least equal to the thickness of the drug-containing solid.

In some embodiments, one or more interconnected free spaces terminate at the outer surface of the drug-containing solid.

In some embodiments, the free space is contiguous.

In some embodiments, the effective free spacing between segments across one or more interconnected free spaces on average is in the range of 1 μm-2.5 mm.

In some embodiments, the free spacing between segments of the one or more structural elements is precisely controlled across the drug-containing solid.

In some embodiments, the three dimensional structural framework comprises a single continuous structure through the drug-containing solid.

In some embodiments, the volume fraction of structural elements within the drug-containing solid is in the range between 0.2 and 0.98 (e.g., 0.25-0.98 or 0.3-0.98).

In some embodiments, the three dimensional structural framework comprises criss-crossed stacked layers of fibers.

In some embodiments, the solubility of physiological fluid in at least one absorptive excipients is greater than 700 mg/ml (e.g., greater than 775 mg/ml, or greater than 825 mg/ml).

In some embodiments, rate of penetration of physiological/body fluid into an absorptive excipient under physiological conditions is greater than the average thickness of the elements divided by 3600 seconds.

In some embodiments, at least one absorptive excipient comprises hydroxypropyl methylcellulose.

In some embodiments, the molecular weight of said hydroxypropyl methyl cellulose excipient is in the range between 45 kg/mol and 500 kg/mol.

In some embodiments, at least one absorptive excipient is selected from the group comprising hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, or vinylpyrrolidone-vinyl acetate copolymer.

In some embodiments, the molecular weight of at least one absorptive excipient is in the range of 50 kg/mol to 10,000 kg/mol.

In some embodiments, the solubility of a relevant physiological fluid in at least a strength-enhancing excipient is no greater than 750 mg/ml under physiological conditions.

In some embodiments, at least a strength-enhancing excipient comprises an elastic modulus in the range of 0.5 MPa-100 MPa after soaking with a physiological fluid under physiological conditions.

In some embodiments at least a strength-enhancing excipient comprises a tensile strength in the range of 0.05 MPa-100 MPa after soaking with a physiological fluid under physiological conditions.

In some embodiments, at least a strength-enhancing excipient comprises a strain at fracture greater than 0.5 after soaking with a physiological fluid under physiological conditions.

In some embodiments, the volume or weight fraction of the one or more absorptive excipients in the fiber is in the range between 0.15 and 0.8.

In some embodiments, the volume or weight fraction of the one or more strength-enhancing excipients in the fiber is in the range between 0.25 and 0.9.

In some embodiments, at least one strength-enhancing excipient comprises an enteric polymer.

In some embodiments, at least one strength-enhancing excipient comprises an enteric polymer, said enteric polymer having a solubility at least 10 times greater in basic solution having a pH value greater than 7 than in acidic solution having a a pH value no greater than 5.

In some embodiments, at least one strength-enhancing excipient comprises methacrylic acid-ethyl acrylate copolymer.

In some embodiments, at least one strength-enhancing excipient is selected from the group comprising hydroxypropyl methyl cellulose acetate succinate, polyvinyl acetate, ethyl acrylate polymers (e.g., polymers including ethyl acrylate), methacrylate polymers (e.g., polymers including methacrylate), ethyl acrylate-methylmethacrylate copolymers, Poly[Ethyl acrylate, methyl methacrylate, trimethylammonioethyl methacrylate chloride], Poly[Ethyl acrylate, methyl methacrylate, trimethylammonioethyl methacrylate chloride], and ethylcellulose.

In some embodiments, said at least two excipients form a solid solution through the thickness of the fiber.

In some embodiments, one or more phases comprising strength-enhancing excipient are substantially connected or substantially contiguous along the length of the fiber.

In some embodiments, an element or framework comprises a plurality of segments having substantially the same weight fraction of physiological fluid-absorptive excipient distributed within the segments.

In some embodiments, an element or framework comprises a plurality of segments having substantially the same weight fraction of strength-enhancing excipient distributed within the segments.

In some embodiments, upon exposure to a physiological fluid under physiological conditions, the diffusivity of absorptive polymeric excipient through said fiber is no greater than 10⁻¹² m²/s (e.g., no greater than 0.5×10⁻¹² m²/s, or no greater than 0.2×10⁻¹² m²/s).

In some embodiments, upon exposure to a physiological fluid under physiological conditions, the diffusivity of said physiological fluid through said fiber is greater than 0.2×10⁻¹² m²/s (e.g., greater than 0.5×10⁻¹² m²/s, or greater than 10⁻¹² m²/s).

In some embodiments, at least one free space is filled with matter removable by a physiological fluid under physiological conditions.

In some embodiments, upon immersion in a physiological fluid, the drug-containing solid transitions to a semi-solid mass comprising a length in the range between 1.3 and 3.5 times its length prior to exposure to said physiological fluid within no more than 300 minutes of immersion in said physiological fluid.

In some embodiments, upon immersion in a physiological fluid, the drug-containing solid transitions to a semi-solid mass comprising a length in the range between 1.3 and 3.5 times its length prior to exposure to said physiological fluid within no more than 100 minutes of immersion in said physiological fluid.

In some embodiments, said expanded fiber or semi-solid mass maintains its length between 1.3 and 4 times the initial length for prolonged time.

In some embodiments, an expanded semi-solid mass comprises an elastic modulus in the range of 0.002 MPa-10 MPa.

In some embodiments, an expanded semi-solid mass comprises a tensile strength in the range between 0.001 MPa and 10 MPa.

In some embodiments, eighty percent of the drug content is released from the drug containing solid into a physiological fluid within 1 hour to 30 days after immersion of the drug-containing solid into said physiological fluid under physiological conditions.

In some embodiments, eighty percent of the drug content is released from the drug containing solid into a physiological fluid within 2 hours to 150 hours after immersion of the drug-containing solid into said physiological fluid under physiological conditions.

In some embodiments, upon ingestion by a human or animal subject, said dosage form is gastroretentive.

Elements of embodiments described with respect to one aspect of the invention can be applied with respect to another aspect. By way of example but not by way of limitation, certain embodiments of the claims described with respect to the first aspect can include features of the claims described with respect to the second or third aspect, and vice versa.

This invention may be better understood by reference to the accompanying drawings, attention being called to the fact that the drawings are primarily for illustration, and should not be regarded as limiting.

### BRIEF DESCRIPTION OF THE DRAWINGS

The objects, embodiments, features, and advantages of the present invention are more fully understood when considered in conjunction with the following accompanying drawings:

FIG. 1 presents schematics of the passage of dosage forms through the gastrointestinal tract: (a) conventional dosage form, and (b) expandable fibrous dosage form. The symbols are designated as follows: t is the time; t₀ is the time when the dosage form enters the stomach; t₁, t₂, and t₃ are specific times after the dosage form entered the stomach where t₁<t₂<t₃; t_{dis} is the time when the expandable fibrous dosage form disintegrates; tₜᵣ is the gastrointestinal transit time;

FIG. 2 shows a non-limiting example of an element for dosage form construction or fabrication as disclosed herein, and the expansion and microstructural evolution upon immersion in a dissolution fluid; t₁, t₂, and t₃ are specific times after immersion of the element in the dissolution fluid, where t₁<t₂<t₃;

FIG. 3 shows a non-limiting example of a pharmaceutical dosage form according to the invention herein and the expansion and drug release processes upon immersion in a dissolution fluid;

FIG. 4 shows another non-limiting example of a pharmaceutical dosage form according to the invention herein and the expansion and drug release processes upon immersion in a dissolution fluid;

FIG. 5 illustrates a non-limiting course of a dosage form herein after ingestion by a human or animal subject at the following times: (a) t=t₀, (b) t=t₁>t₀, (c) t=t₂>t₁, and (d) t≈t_{dis}. The times are designated as follows: t₀ is time when dosage form enters the stomach and t_{dis} is time when dosage form disintegrates or fragments;

FIG. 6 shows another non-limiting example of a pharmaceutical dosage form according to the invention herein and the expansion and drug release processes upon immersion in a dissolution fluid;

FIG. 7 presents non-limiting schematics of water absorption and water concentration in the fiber: (a) initial solid fiber comprising a solid solution of sparingly-soluble drug, absorptive excipient (HPMC), and strength-enhancing excipient (an enteric excipient), and (b) semi-solid or viscous fiber at time t after immersion in acidic water. Because the solubility of acidic water in HPMC is high but low in the enteric excipient and very small in the drug, the HPMC-enteric excipient-drug-water solution may separate out into three phases: (1) a highly viscous solution of water, HPMC, and dissolved drug molecules, (2) water-plasticized enteric excipient, and (3) drug particles;

FIG. 8 shows a non-limiting schematic microstructure of an expanding dosage form: (a) initial structure and (b) structure at time t after exposure to a physiological or dissolution fluid;

FIG. 9 presents non-limiting schematics of drug release by expanded fibrous dosage forms: (a) φ˜0, (b) 0<φ<1, and (c) φ˜1. φ is the volume fraction of fibers in the dosage form. The time increases towards the right in the sequences of the schematics;

FIG. 10 is a non-limiting schematic illustrating drug release from an expanded fiber containing both drug particles and drug molecules: (a) expanded fiber at a specific time after immersion in a stirred dissolution fluid, (b) drug concentration versus radius, r, assuming an infinitesimally thin interfacial region and a quasi-steady concentration profile in the particle-depleted region. Also shown are: particle-dispersed region (A), interfacial region (B), particle-depleted region (C) in the fiber, and the dissolution fluid (D) outside the fiber;

FIG. 11 shows a non-limiting schematic of drug release from an expanded, semi-solid dosage form with 2R/λ˜1: (a) dosage form at a specific time after immersion in a stirred dissolution fluid, (b) drug concentration versus distance, x, assuming a quasi-steady concentration profile in the particle-depleted region. A: particle-dispersed region, B: interfacial region, C: particle-depleted region, and D: dissolution fluid;

FIG. 12 shows a non-limiting schematic of an expanded, semi-solid dosage form exposed to cyclic loading;

FIG. 13 presents a non-limiting dosage form according to the invention herein along with its microstructure;

FIG. 14 presents a non-limiting fibrous microstructure of a dosage form herein, and a histogram of the length of fiber segments between adjacent contacts;

FIG. 15 shows another non-limiting fibrous microstructure herein, and a histogram of the angle between contacting fibers;

FIG. 16 presents a non-limiting example of a point contact between elements or segments;

FIG. 17 is a non-limiting example of a line contact between elements or segments;

FIG. 18 presents non-limiting microstructures of elements herein prior and after exposure to a physiological fluid: (a) solid solution of drug molecules, absorptive excipient, and strength-enhancing excipient prior exposure to a physiological fluid, (b) solid solution of drug molecules, absorptive excipient, and strength-enhancing excipient after exposure to a physiological fluid, (c) core-shell structure comprising a core of drug and absorptive excipient, and a shell of strength-enhancing excipient prior exposure to a physiological fluid, (d) core-shell structure comprising a core of drug and absorptive excipient, and a shell of strength-enhancing excipient after exposure to a physiological fluid, (e) dispersed particles of absorptive excipient and drug in a matrix of strength-enhancing excipient prior exposure to a physiological fluid, (f) dispersed particles of absorptive excipient and drug in a matrix of strength-enhancing excipient after exposure to a physiological fluid, (g) dispersed particles of strength-enhancing excipient in a matrix of drug and absorptive excipient prior exposure to a physiological fluid, and (h) dispersed particles of strength-enhancing excipient in a matrix of drug and absorptive excipient after exposure to a physiological fluid;

FIG. 19 shows a non-limiting example of a dosage form as disclosed herein and its expansion and drug release after exposure to a physiological fluid;

FIG. 20 presents scanning electron micrographs of a non-limiting single fiber and a non-limiting fibrous dosage form according to the invention herein;

FIG. 21 shows images of a single fiber at various times after immersion in a dissolution fluid. The fiber transitioned from solid to viscous and swelled both radially and axially;

FIG. 22 presents experimental results of the expansion of single fibers after immersion in a dissolution fluid: (a) normalized radial expansion, ΔR/R₀, versus time after immersion, t, (b) normalized axial expansion, ΔL/L₀, versus t, (c) ΔR/R₀, versus t^{1/2}, and (d) ΔL/L₀ versus t^{1/2};

FIG. 23 presents experimentally-derived images of the fibrous dosage forms after immersion in a dissolution fluid. The volume fractions of fibers in the solid dosage forms, φ, were: (a) φ=0.16, (b) φ=0.39, and (c) φ=0.56;

FIG. 24 plots experimental results of the normalized longitudinal expansion, ΔL/L₀, of fibrous dosage forms after immersion in a dissolution fluid: (a) ΔL/L₀ versus time, t, after immersion, and (b) ΔL/L₀ versus t^{1/2}/R₀;

FIG. 25 displays experimental results of drug release by single fibers after immersion in a dissolution fluid: (a) fraction of drug released, m_{d}/M₀, versus time, t, after immersion and (b) m_{d}/M₀ versus t^{1/2}/R₀;

FIG. 26 presents experimental results of drug release by fibrous dosage forms after immersion in a dissolution fluid: (a) fraction of drug released, m_{d}/M₀, versus time after immersion, t, and (b) measured data and calculated curves of m_{d}/M₀ versus t^{1/2}. The calculated curves were obtained by Eq. (43) using cₛ=0.05 mg/ml, c_{d,0}=37.9 mg/ml, D_{d}=3.24×10⁻¹⁰ m²/s, R=83 μm, and H=2 mm.

FIG. 27 shows a semi-log plot of t_{0.8} versus φ. The straight line is: t_{0.8}=0.77×exp(7.06φ);

FIG. 28 presents scanning electron micrographs of dosage forms dip-coated with enteric excipient: (a) Low-magnification image of top and (b) front views of the microstructure, and (c) high-magnification image of the cross-section of a coated fiber;

FIG. 29 shows top-view images of dosage forms after immersion in a dissolution fluid: (a) uncoated dosage form, and (b) enteric coated dosage form;

FIG. 30 plots the normalized radial expansion of the dosage forms, ΔR_{df}/R_{df,0}, versus time, t, after immersion in the dissolution fluid;

FIG. 31 shows images of expanded, fluid-soaked dosage forms during diametral compression: (a) uncoated and (b) coated dosage form;

FIG. 32 presents results of the diametrial compression test of expanded, fluid-soaked dosage forms: (a) load per unit length, P, versus displacement, δ, of uncoated and coated dosage forms and (b) dP/dδ versus δ. The inset of FIG. 6a shows a schematic of the loads applied on a homogeneous, isotropic, linear elastic cylinder compressed by diametrically opposed flat platen. P is the load intensity or force per unit thickness. R_{df} is the radius of the cylinder (or expanded dosage form). The small arrows represent the Hertzian contact pressure distributed over the contact width 2a;

FIG. 33 shows images of an expanded, coated dosage form before (left) and after diametral compression (right). The compression-tested coated dosage form had visible cracks within the axis of symmetry;

FIG. 34 depicts the position and structure of an uncoated dosage form after administration to a fasted dog. Dry food was given 4-6 hours after administration; it is visible in the bottom row images. The images were obtained by biplanar fluoroscopy. They show the abdomen in lateral projection (cranial left, caudal right);

FIG. 35 depicts the position and structure of a coated dosage form after administration to a fasted dog. Dry food was given 4-6 hours and 30 hours after administration. The images were obtained by biplanar fluoroscopy. They show the abdomen in lateral projection (cranial left, caudal right);

FIG. 36 Expansion of dosage form radius in vivo and comparison with in vitro data: (a) uncoated and coated dosage forms in vivo, and (b) in vivo/in vitro comparison of uncoated and coated dosage forms;

FIG. 37 depicts fluoroscopic image sequences during contraction pulsing by the stomach walls: (a) uncoated dosage form in the stomach 2 hours after administration, and (b) coated dosage form in the stomach 7 hours after administration;

FIG. 38 presents experimental results of the sorption of physiological fluid by a strength-enhancing excipient herein;

FIG. 39 plots the nominal tensile stress, σ, versus engineering strain, ε, in thin, acidic water-soaked tensile specimen films of Eudragit L100-55. The stress was derived as: σ=F/Wh where F is the force applied by the grips, W the width of the thin section of the specimen film, and h its thickness. The engineering strain, ε=ΔL/L₀ where ΔL is the distance travelled by the grips and L₀ the initial distance between grips.

FIG. 40 presents non-limiting schematics of gastro-intestinal passage of drug, and drug concentration in blood versus time. Top row: granular dosage form. Bottom row: expandable fibrous dosage form.

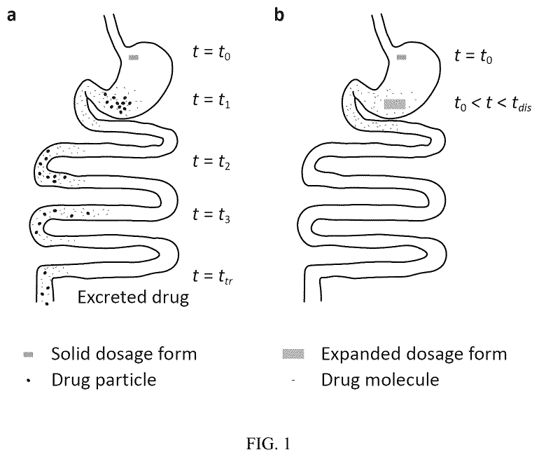

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Moreover, in the disclosure herein, the terms “one or more active ingredients” and “drug” are used interchangeably. As used herein, an “active ingredient” or “active agent” or “drug” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

Furthermore, in the context of some embodiments herein, a three dimensional structural framework (or network) of one or more elements comprises a drug-containing structure (e.g., an assembly or an assemblage or an arrangement or a skeleton or a skeletal structure or a three-dimensional lattice structure of one or more drug-containing elements) that extends over a length, width, and thickness greater than 100 μm. This includes, but is not limited to drug-containing structures that extend over a length, width, and thickness greater than 200 μm, or greater than 300 μm, or greater than 500 μm, or greater than 700 μm, or greater than 1 mm, or greater than 1.25 mm, or greater than 1.5 mm, or greater than 2 mm.

In other embodiments, a three dimensional structural framework (or network) of drug-containing elements may comprise a drug-containing structure (e.g., an assembly or an assemblage or a skeleton or a skeletal structure of one or more elements) that extends over a length, width, and thickness greater than the average thickness of at least one element (or at least one segment) in the three dimensional structural framework (or network) of elements. This includes, but is not limited to drug-containing structures that extend over a length, width, and thickness greater than 1.5, or greater than 2, or greater than 2.5, or greater than 3, or greater than 3.5, or greater than 4 times the average thickness of at least one element (or at least one segment) in the three dimensional structural framework (or network) of elements.

In some embodiments, a three dimensional structural framework (or network) of drug-containing elements is continuous. Furthermore, in some embodiments, the drug-containing elements are bonded to each other or interpenetrating.

It may be noted that the terms “three dimensional structural network”, “three dimensional structural framework”, and “three dimensional lattice structure” are used interchangeably herein. Also, the terms “three dimensional structural framework of drug-containing elements”, “three dimensional structural framework of elements”, “three dimensional structural framework of one or more elements”, “three dimensional structural framework of one or more drug-containing elements”, “three dimensional framework of elements”, “three dimensional structural framework of fibers”, “three dimensional framework”, “structural framework”, etc. are used interchangeably herein.

In the invention herein, a “structural element” or “element” refers to a two-dimensional element (or 2-dimensional structural element), or a one-dimensional element (or 1-dimensional structural element), or a zero-dimensional element (or 0-dimensional structural element).

As used herein, a two-dimensional structural element is referred to as having a length and width much greater than its thickness. In the present disclosure, the length and width of a two-dimensional structural element are greater than 2 times its thickness. An example of such an element is a “sheet”. A one-dimensional structural element is referred to as having a length much greater than its width or thickness. In the present disclosure, the length of a one-dimensional structural element is greater than 2 times its width and thickness. An example of such an element is a “fiber”. A zero-dimensional structural element is referred to as having a length and width of the order of its thickness. In the present disclosure, the length and width of a zero-dimensional structural element are no greater than 2 times its thickness. Furthermore, the thickness of a zero-dimensional element is less than 2.5 mm. Examples of such zero-dimensional elements are “particles” or “beads” and include polyhedra, spheroids, ellipsoids, or clusters thereof.

Moreover, in the invention herein, a segment of a one-dimensional element is a fraction of said element along its length. A segment of a two-dimensional element is a fraction of said element along its length and/or width. A segment of a zero-dimensional element is a fraction of said element along its length and/or width and/or thickness. The terms “segment of a one-dimensional element”, “fiber segment”, “segment of a fiber”, and “segment” are used interchangeably herein. Also, the terms “segment of a two-dimensional element” and “segment” are used interchangeably herein. Also, the terms “segment of a zero-dimensional element” and “segment” are used interchangeably herein.

As used herein, the terms “fiber”, “fibers”, “one or more fibers”, “one or more drug-containing fibers”, and “drug-containing fibers”, are used interchangeably. They are understood as the solid, drug-containing structural elements (or building blocks) that make up part of or the entire three dimensional structural network (e.g., part of or the entire dosage form structure, or part of or the entire structure of a drug-containing solid, etc.). A fiber has a length much greater than its width and thickness. In the present disclosure, a fiber is referred to as having a length greater than 2 times its width and thickness (e.g., the length is greater than 2 times the fiber width and the length is greater than 2 times the fiber thickness). This includes, but is not limited to a fiber length greater than 3 times, or greater than 4 times, or greater than 5 times, or greater than 6 times, or greater than 8 times, or greater than 10 times, or greater than 12 times the fiber width and thickness. In other embodiments that are included but not limiting in the disclosure herein, the length of a fiber may be greater than 0.3 mm, or greater than 0.5 mm, or greater than 1 mm, or greater than 2.5 mm.

Moreover, as used herein, the term “fiber segment” or “segment” refers to a fraction of a fiber along the length of said fiber.

In the invention herein, fibers (or fiber segments) may be bonded, and thus they may serve as building blocks of “assembled structural elements” with a geometry different from that of the original fibers. Such assembled structural elements include two-dimensional elements, one-dimensional elements, or zero-dimensional elements.

In the invention herein, drug release from a solid element (or a solid dosage form, or a solid matrix, or a drug-containing solid) refers to the conversion of drug (e.g., one or more drug particles, or drug molecules, or clusters thereof, etc.) that is/are embedded in or attached to the solid element (or the solid dosage form, or the solid matrix, or three dimensional structural framework, or the drug-containing solid) to drug in a dissolution medium.

A sparingly-soluble drug herein comprises an active ingredient or drug with a solubility in physiological fluid or body fluids (or a dissolution medium or an aqueous solution) smaller than 1 mg/ml under physiological conditions. This includes, but is not limited to a solubility in physiological fluid or body fluid under physiological conditions smaller than 0.5 mg/ml, or smaller than 0.2 mg/ml, or smaller than 0.1 mg/ml, or smaller than 0.05 mg/ml, or even smaller. It may be noted that the terms “sparingly-soluble drug”, “sparingly water-soluble drug”, and “poorly-soluble drug” are used interchangeably herein.

As used herein, the terms “dissolution medium”, “physiological fluid”, “body fluid”, “dissolution fluid”, “medium”, “fluid”, “aqueous solution”, and “penetrant” are used interchangeably. They are understood as any fluid produced by or contained in a human body under physiological conditions, or any fluid that resembles a fluid produced by or contained in a human body under physiological conditions. Generally, a dissolution fluid contains water and thus may be aqueous. Examples include, but are not limited to: water, saliva, stomach fluid, gastrointestinal fluid, saline, etc. at a temperature of 37° C. and a pH value adjusted to the relevant physiological condition.

In the invention herein, moreover, an “absorptive excipient” is referred to as an excipient that is “absorptive” of gastric or a relevant physiological fluid under physiological conditions. Generally, said absorptive excipient is a solid, or a semi-solid, or a viscoelastic material in the dry state at room temperature. Upon contact with (e.g., immersion in) gastric or a relevant physiological fluid under physiological conditions, however, said absorptive excipient can absorb said fluid and form solutions or mixtures with said fluid having a weight fraction of gastric or relevant physiological fluid greater than 0.4. This includes, but is not limited to the formation of solutions or mixtures with a weight fraction of gastric or relevant physiological fluid greater than 0.5, or greater than 0.6, or greater than 0.7, or greater than 0.75, or greater than 0.8, or greater than 0.85, or greater than 0.9, or greater than 0.95. In other words, the solubility of gastric fluid or a relevant physiological fluid in the absorptive excipient under physiological conditions generally is greater than about 400 mg/ml. This includes, but is not limited to solubility of gastric or relevant physiological fluid in an absorptive excipient greater than 500 mg/ml, or greater than 600 mg/ml, or greater than 700 mg/ml, or greater than 750 mg/ml, or greater than 800 mg/ml, or greater than 850 mg/ml, or greater than 900 mg/ml, or greater than 950 mg/ml. Preferably, absorptive excipient is mutually soluble with a relevant physiological fluid. In the invention herein, a “relevant physiological fluid” is understood as the relevant physiological fluid surrounding the dosage form in the relevant physiological application. For example, if the dosage form is a gastroretentive dosage form, a relevant physiological fluid is gastric fluid. Non-limiting examples of preferred absorptive, high-molecular-weight excipients may include, but are not limited to water-soluble polymers of large molecular weight and with amorphous molecular structure, such as hydroxypropyl methylcellulose with a molecular weight greater than 50 kg/mol or hydroxypropyl methylcellulose with a molecular weight in the range between 50 kg/mol and 300 kg/mol. The terms “physiological fluid-absorptive excipient”, “absorptive excipient”, “fluid-absorptive excipient”, and “water-absorptive excipient” are used interchangeably herein.

In the invention herein, moreover, a “strength-enhancing excipient”, too, generally is a solid, or a semi-solid, or a viscoelastic material in the dry state at room temperature. Upon contact with (e.g., immersion in) gastric or a relevant physiological fluid under physiological conditions, however, said strength-enhancing excipient is far less absorptive of said fluid, and thus it remains a semi-solid, or viscoelastic, or highly viscous material. Generally, the solubility of gastric or relevant physiological fluid in strength-enhancing excipient under physiological conditions is no greater than 800 mg/ml. This includes, but is not limited to a solubility of gastric or a relevant physiological fluid in strength-enhancing excipient under physiological conditions no greater than 750 mg/ml, or no greater than 700 mg/ml, or no greater than 650 mg/ml, or no greater than 600 mg/ml, or no greater than 550 mg/ml, or no greater than 500 mg/ml, or no greater than 450 mg/ml, or no greater than 400 mg/ml. In the non-limiting extreme case, the relevant physiological fluid can be insoluble or practically insoluble in the strength-enhancing excipient.

Typically, however, a relevant physiological fluid is sparingly-soluble in a strength-enhancing excipient. Thus, upon immersion of said strength-enhancing excipient in said relevant physiological fluid, the stiffness (e.g., the elastic modulus) or the viscosity of said strength-enhancing excipient may decrease somewhat compared with the stiffness or viscosity of the dry strength-enhancing excipient. Similarly, upon immersion of strength-enhancing excipient in a relevant physiological fluid, the strain at fracture of said strength-enhancing excipient may increase compared with the strain at fracture of the dry strength-enhancing excipient. Because the strength-enhancing excipient can be a semi-solid or viscoelastic or highly viscous material even after prolonged immersion in a relevant physiological fluid, it is also referred to herein as “stabilizing excipient”, “viscoelastic excipient”, or “semi-solid excipient”.

In the invention herein, moreover, a “solid solution” of at least two constituents (e.g., at least two excipients) is referred to as a solid having at least two constituents that are partially or entirely dissolved (e.g., molecularly dispersed or molecularly mixed) in each other. This includes, but is not limited to a first constituent (e.g., a first excipient) that is dissolved or molecularly dispersed or molecularly mixed in a second constituent (e.g., a second excipient), or a second constituent that is dissolved or molecularly dispersed or molecularly mixed in a first constituent. The solid solution may have a molecular arrangement or crystal structure that is the same or similar to that of the first constituent, or it may have a molecular arrangement or crystal structure that is the same or similar to that of the second constituent, or it may have a molecular arrangement or crystal structure that is different from that of the first constituent and also different from that of the second constituent. Often times, however, the at least two constituents are amorphous polymers, and the resulting solid solution is an amorphous polymer, too. Often times, moreover, and in preferred embodiments, the concentrations of the at least two molecularly dispersed constituents forming the solid solution are substantially uniform across the solid solution. By way of example but not by way of limitation, in a solid material a solid solution may be experimentally detected or shown by such methods as Differential Scanning calorimetry, x-ray spectroscopy, Fourier-transform infrared spectroscopy, Raman spectroscopy, and so on. For further information related to solid solutions, see, e.g., the International Application No. PCT/US19/52030 filed on Sep. 19, 2019 and titled “Dosage form comprising structured solid-solution framework of sparingly-soluble drug and method for manufacture thereof” and any references therein.

Further information related to the definition, characteristics, features, composition, analysis etc. of the disclosed dosage forms, and the elements for fabricating or constructing them, is provided throughout this specification.

### Scope of the Invention

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

By way of example but not by way of limitation, it is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Furthermore, where compositions, articles, and devices are described as having, including, or comprising specific components, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. Headers are provided for organizational purposes and are not meant to be limiting

### DETAILED DESCRIPTION OF THE INVENTION

### Aspects of an Element

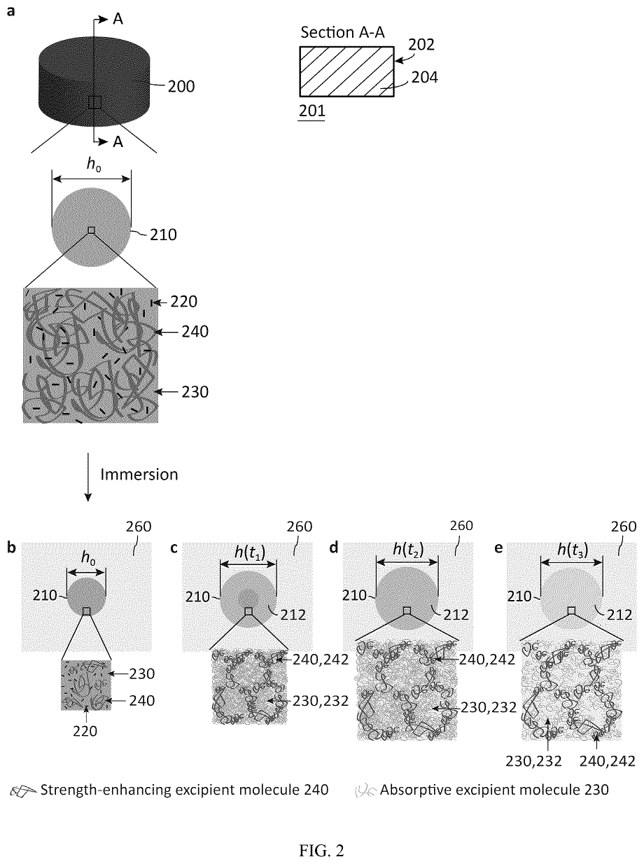

As shown schematically in FIG. 2a, the dosage forms 200 disclosed herein generally comprise a drug-containing solid 201 having an outer surface 202 and an internal structure 204 including one or more elements 210. A non-limiting example of a single element 210, such as a fiber 210, for pharmaceutical dosage form construction or fabrication is illustrated in the insets of FIG. 2a. The fiber or element 210 includes at least one active ingredient 220 and at least two excipients 230, 240 forming the element or fiber 210. The at least two excipients 230, 240 comprise one or more fluid-absorptive polymeric constituents 230 and one or more strength-enhancing polymeric constituents 240.

Upon exposure to physiological fluid 260, such as saliva, gastric fluid, a fluid that resembles a physiological fluid, and so on, the one or more strength-enhancing excipients 240 form a fluid-permeable, semi-solid network 242 to mechanically support the element or fiber 210, FIGS. 2b-2e. Also, the one or more fluid-absorptive excipients 230 transition to a viscous mass, or a viscous solution 232, expanding said element or fiber 210 along at least one dimension with absorption of said physiological fluid 260.

FIG. 2a presents a further non-limiting example of a single element 210, such as a fiber 210, for pharmaceutical dosage form construction or fabrication. The element or fiber 210 includes at least one active ingredient 220 and at least two excipients 230, 240 forming the element or fiber 210. The at least two excipients 230, 240 comprise one or more fluid-absorptive polymeric constituents 230, within which the solubility of a physiological fluid is greater than 600 mg/ml. The at least two excipients 230, 240 further comprise one or more strength-enhancing polymeric constituents 240. The one or more strength-enhancing polymeric constituents 240 have an elastic modulus in the range between 0.1 MPa and 200 MPa and a strain at fracture greater than 0.5 after soaking with said physiological fluid under physiological conditions. Preferably, moreover, the one or more strength-enhancing polymeric constituents 240 form one or more phases that are substantially connected and/or substantially contiguous along length of the element or fiber 210. Generally, a “phase” is understood herein as a region or space within an element or fiber 210 throughout which many or all physical properties are substantially uniform or constant. By way of example but not by way of limitation, a solid solution including strength-enhancing excipient 240 comprises a “phase comprising one or more strength-enhancing excipients”.

Upon exposure to said physiological fluid 260, said fluid 260 diffuses into said element or fiber 210, thereby expanding said element or fiber 210 in at least one dimension to a length between 1.3 and 4 times its length prior to exposure to said physiological fluid 260, FIGS. 2b and 2c. By way of example but not by way of limitation, the initial thickness of said element or fiber 210 (also referred to herein as the “thickness prior to exposure to said physiological fluid”), h₀, may expand to a length, h=1.3-4 times h₀, upon exposure to said physiological fluid 260.

Also, upon exposure to said physiological fluid 260, said element or fiber 210 transitions to a semi-solid mass 212. The semi-solid mass 212 may maintain its length between 1.3 and 4 times the initial length for prolonged time, FIGS. 2c-2e. The term “the semi-solid mass maintains its length between 1.3 and 4 times the initial length for prolonged time” is referred to herein as a semi-solid mass immersed in an unstirred or lightly stirred dissolution fluid (e.g., acidic water), wherein said immersed semi-solid mass maintains its length between 1.3 and 4 times the initial length for an extended time, such as a time greater than 1 hour, or a time greater than 2 hours, or a time greater than 5 hours, or an even longer time.

Moreover, upon exposure to said physiological fluid, the one or more strength-enhancing excipients 240 form a fluid-permeable, semi-solid network 242 within or through the element or fiber 210 or semi-solid mass 212 to mechanically support the element or fiber or semi-solid mass 210, 212. Also, the one or more fluid-absorptive excipients 230 transition to a viscous mass, or a viscous solution 232, expanding said element or fiber 210 in at least one dimension with absorption of said physiological fluid 260.

It may be noted that generally, one or more strength-enhancing excipients form a “fluid-permeable, semi-solid network to mechanically support the element or fiber or semi-solid mass” within or through an element, if the mechanical strength or stiffness (e.g., the elastic modulus, or the tensile strength, etc.) of said element after exposure to a physiological fluid is substantially greater than the mechanical strength or stiffness of an element comprising fluid-absorptive excipient alone (e.g., no strength-enhancing excipient) after exposure to said physiological fluid. By way of example but not by way of limitation, one or more strength-enhancing excipients form a “fluid-permeable, semi-solid network to mechanically support the element or fiber or semi-solid mass” within or through an element, if the tensile strength or the elastic modulus of said element after exposure to a physiological fluid is at least two times greater than that of a corresponding element comprising fluid-absorptive excipient alone (e.g., no strength-enhancing excipient) after exposure to said physiological fluid. This includes, but is not limited to the tensile strength or the elastic modulus of an element with one or more strength-enhancing excipients forming a “fluid-permeable, semi-solid network to mechanically support the element or fiber or semi-solid mass” after exposure to a physiological fluid at least three times greater, or at least four times greater, or at least five times greater, or at least six times greater, or at least seven times greater than that of a corresponding element comprising fluid-absorptive excipient alone (e.g., no strength-enhancing excipient) after exposure to said physiological fluid.

Additional aspects and embodiments of structural elements or fibers according to the invention herein are described throughout this specification. Any more aspects and embodiments of structural elements or fibers obvious to a person of ordinary skill in the art are all within the spirit and scope of this invention.

### Aspects of the Dosage Form

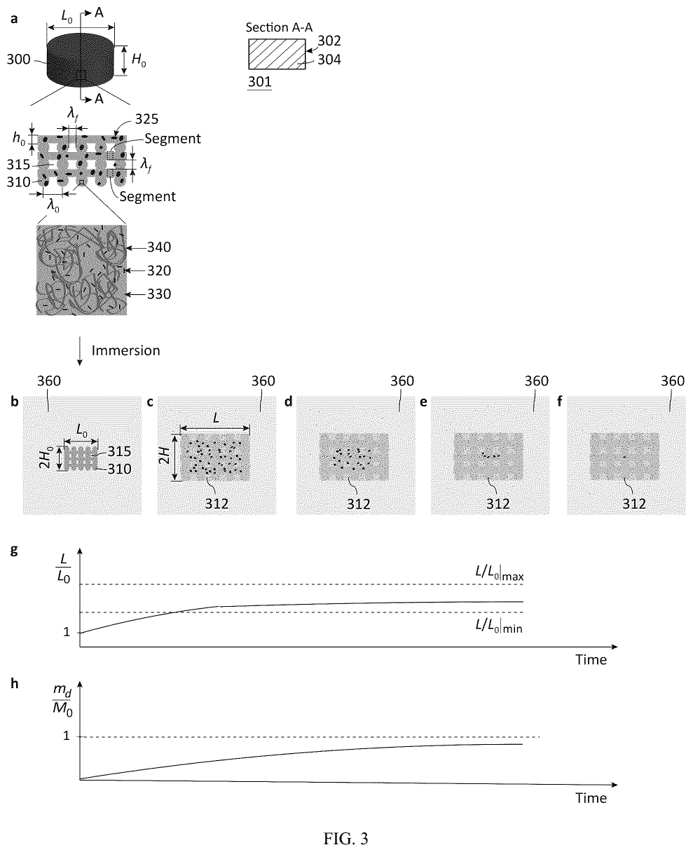

FIG. 3a presents a non-limiting example of a pharmaceutical dosage form disclosed herein. The dosage form 300 comprises a drug-containing solid 201 having an outer surface 302 and an internal three dimensional structural framework 304 (e.g., a lattice structure, a network, a skeleton, etc.) of one or more thin, structural elements 310. In the invention herein, a structural element is understood “thin” if its thickness (e.g., its smallest dimension) is much smaller than the length, or width, or thickness of the dosage form. Thin, structural elements 310 are also referred to herein as “elements” or “structural elements”. The structural elements 310 may comprise fibers, beads, sheets, or combinations thereof.

The framework 304 is contiguous with and terminates at said outer surface 302. In preferred embodiments, the structural framework 304 forms a single continuous or connected structure through the drug-containing solid 301 (e.g., in this case all elements 310 may be bonded to at least another element 310 to form a single continuous structure). In preferred embodiments, moreover, the one or more thin structural elements 310 are orderly or substantially orderly arranged.

The elements 310 further comprise segments spaced apart from segments of adjoining elements or segments, thereby defining one or more free spaces 315 within the drug-containing solid 301. In preferred embodiments, the free spaces 315 are interconnected through or across the drug-containing solid 301. In the invention herein, a free space 315 may generally be referred to as “interconnected through or across the drug-containing solid” if it extends (e.g., if the free space 315 is continuous or connected) over a length at least half the thickness of the drug-containing solid 301. This includes, but is not limited to free space 315 extending over a length at least two-third the thickness of the drug-containing solid 301, or free space 315 extending over a length at least equal to the thickness of the drug-containing solid 301. A free space 315 may also be considered interconnected across or through the drug-containing solid 301 herein if it extends over a length at least twice the thickness of one or more elements 310. Furthermore, in preferred embodiments one or more interconnected free spaces 315 are connected to the outer surface 302. Thus no walls (e.g., walls comprising the three dimensional structural framework 304 of elements 310) must be ruptured to obtain an interconnected free space 315 (e.g., an open channel of free space 315) from the outer surface 302 of the drug-containing solid 301 to a point or position (or to any point) in said interconnected free space 315. Generally, moreover, at least one of said one or more interconnected free spaces 315 is filled with matter removable by a physiological fluid under physiological conditions (e.g., a gas, a solid that is highly soluble in said physiological fluid, etc.).

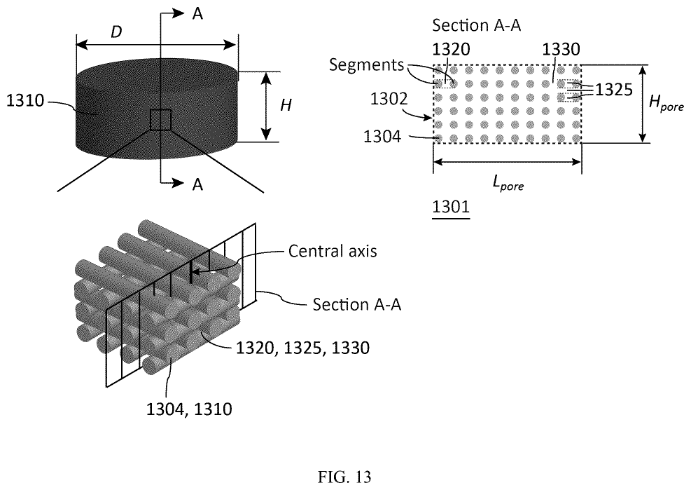

A non-limiting example of a preferred internal three dimensional structural framework 304 comprises a plurality of criss-crossed stacked layers of fibrous elements 310. Herein criss-crossed stacked layers of fibrous elements 310 are referred to as plies (e.g., “layers” or “planes”) of fibers 310 or fiber segments that are stacked in a cross-ply arrangement. In cross-ply arrangements, fibers 310 (or fiber segments) in a ply (or “layer” or “plane”) are oriented transversely or at an angle to the fibers 310 in the ply above or below. Moreover, in cross-ply structures the free space 315 typically extends over the entire length, width, and thickness of the drug-containing solid 301. More so, the free space 315 is contiguous and terminates at the outer surface 302 of the drug-containing solid 301. Further details about how interconnected free spaces 315 are defined herein, what they may be composed of, and how their length may be measured are provided in FIG. 13 herein, and in section “Embodiments of the dosage form”.

In the invention herein, moreover, the structural elements 310 comprise at least an active ingredient 320, 325 (e.g., at least a drug) and at least two excipients 330, 340 (also referred to herein as “dual excipient”). Typically, the at least one active ingredient 320, 325 is dispersed in at least one of said at least two excipients 330, 340 as active ingredient molecules 320 or as particles 325 comprising said at least one active ingredient. Thus, the at least two excipients 330, 340 (or all excipients, or the excipient in its totality) may form a continuous or connected structure through one or more elements 310 (e.g., through the thickness of one or more elements, and/or through the length of one or more elements, and/or through the width of one or more elements) or through the three-dimensional structural framework 304. In some preferred embodiments, moreover, said at least two excipients 330, 340 may form a solid solution.

Said at least two excipients 330, 340 further comprise at least a physiological fluid-absorptive polymeric constituent 340 (e.g., a water-absorptive polymeric constituent) and at least a strength-enhancing polymeric constituent 340.

As shown schematically in the non-limiting FIG. 3b, upon immersion of the dosage form 300 or drug-containing solid 301 in a dissolution fluid 260 (e.g., acidic water, gastric fluid, a relevant physiological fluid, a fluid that resembles a relevant physiological fluid, etc.), the fluid 360 may percolate or access interconnected free space 315, and wet the structural framework 304, 310. In the invention herein, a surface (e.g., a surface of the three dimensional structural framework 304, 310) is “wetted by a fluid” if said fluid contacts (e.g., is in contact with) said surface. Moreover, a surface is generally understood herein as “uniformly wetted” by a fluid if at least 20-70 percent of the area of said surface is in contact (e.g., in direct contact) with said fluid. In preferred embodiments herein, upon immersion of the drug-containing solid 301 in a physiological fluid 360, at least 60-70 percent of the surface of the three dimensional structural framework 304, 310 (or a coating of the three dimensional structural framework 304, 310) is wetted by (e.g., contacted by) said fluid at a time in the range between the time of immersion and 600 seconds after the time of immersion.

The fluid 360 may then diffuse or penetrate into the three dimensional structural framework 304 or the elements 310 or segments it surrounds. Moreover, as water or dissolution fluid or physiological fluid 360 diffuses into the elements 310, and the fluid 360 mass and volume in the elements 310 increases, they may expand. In some embodiments, therefore, the drug-containing solid 301 or the three-dimensional structural framework 304, 310 expand due to the penetration (e.g., the diffusion or inflow) of physiological or body fluid 360 into the three dimensional structural framework of elements 304, 310.

It may be noted that within or through the one or more elements or framework 210, 304, 310, as was shown schematically in FIGS. 2c-2e, the one or more strength-enhancing excipients 240 may form a fluid-permeable, semi-solid network 242 to mechanically support the elements or framework 210, 304, 310. Also, the one or more fluid-absorptive excipients 230, 330 may transition to a viscous mass, or a viscous solution 232, expanding said element or framework 210, 304, 310 in at least one dimension with absorption of said physiological fluid 260, 360.

Moreover, if the three-dimensional structural framework 304, 310 is uniformly wetted, and the composition and geometry (e.g., the thickness of the elements, etc.) are substantially uniform across the three-dimensional structural framework 304, 310, the drug-containing solid 301 or three-dimensional structural framework 304, 310 may expand uniformly and in all dimensions as shown schematically in the non-limiting FIG. 3c. The terms “expanding in all dimensions”, “expand in all dimensions”, or “expansion in all dimensions” are understood as an increase in a length of a sample (e.g., the length, and/or width, and/or thickness, etc. of said sample) and an increase in volume of said sample. Thus, pure shear deformation is not considered “expansion in all dimensions” herein.

The expansion of the dosage form 300 or drug-containing solid 301 can be quite substantial, as shown schematically in FIGS. 3b and 3c. Thus, in some embodiments, at least one dimension of the drug-containing solid 301, 304 (e.g., a side length of the drug-containing solid or framework 301, 304, the thickness of the drug-containing solid or framework 301, 304, etc.) expands to at least 1.3 times the initial value (e.g., the initial length or the length prior to exposure to said physiological fluid 360) upon immersion in said physiological fluid 360. This includes, but is not limited to at least one dimension of the drug-containing solid expanding to at least 1.35 times, or at least 1.4 times, or at least 1.45 times, or at least 1.5 times, or at least 1.55 times, or at least 1.6 times, or at least 1.65 times, or at least 1.7 times, or at least 1.75 times, or at least 1.8 times, or at least 1.85 times, or at least 1.9 times the initial value upon immersion in or exposure to a dissolution fluid 360.

Furthermore, in some embodiments the dosage form 300 or drug-containing solid or framework 301, 304 expands to at least 2 times its initial volume upon immersion in or upon exposure to a physiological fluid 360 under physiological conditions. This includes, but is not limited to a drug-containing solid or framework 301, 304 expanding to at least 2.5 times, or at least 3 times, or at least 3.5 times, or at least 4 times, or at least 4.5 times, or at least 5 times, or at least 5.5 times its initial volume upon immersion in or exposure to a physiological fluid 360.

The rate of expansion generally depends on the rate at which physiological fluid 360 is absorbed by the structural framework 304, 310 (e.g., by one or more absorptive polymeric excipients 330, etc.), and the presence and stringency of constraints to expansion. The absorption rate of physiological fluid 360 by the framework 304, 310 is typically increased if the specific surface area (e.g., the surface area to volume ratio) of the framework 304, 310 is increased. Thus, if the elements 310 are thin, the surface area to volume ratio is typically large, and the rate at which physiological fluid 360 is absorbed by the framework 304, 310 can be fast.

Constraints to expansion may, for example, originate from non-uniformities in the physiological fluid 360 concentration across the three dimensional structural framework 304, 310. By way of example but not by way of limitation, a wet element or segment may absorb physiological fluid, but expansion of said wet element or segment may be constrained if it is connected (e.g., attached) to a dry solid element or segment that does not expand. Thus, to minimize constraints to expansion, uniform wetting of elements in the structural framework can be crucial. Uniform wetting is enabled, among others, by interconnected free spaces (e.g., by a continuous free space through which physiological fluid may percolate), and by a hydrophilic surface composition of the three-dimensional structural framework of elements.

The expansion of one or more elements 310 or of the framework 304 or of the drug-containing solid 301 may also be constrained if the stiffness, or an elastic modulus, or a plastic modulus of a strength-enhancing excipient 340 network within an element or fiber 310 is too large. Thus, after exposure to a physiological fluid 360, the stiffness, or an elastic modulus, or a plastic modulus of one or more strength-enhancing excipients 340 should generally not be too large, so that expansion of the dosage form (or of the drug-containing solid 301, or of the framework 304, or of one or more elements 310) is not excessively constrained. However, the stiffness, or an elastic modulus, or a plastic modulus of one or more strength-enhancing excipients 340 should also be large enough to ensure that the strength-enhancing excipient 340 network mechanically supports or stabilizes the one or more elements or framework 304, 310 sufficiently after exposure to a physiological fluid 360. Preferably, therefore, after soaking with physiological fluid 360 under physiological conditions, the one or more strength-enhancing polymeric constituents 340 have an elastic modulus in the range between 0.1 MPa and 200 MPa.

Similarly, to ensure that semi-solid strength-enhancing excipient network does not fracture upon expansion, and is highly connected in an expanded element or semi-solid mass, one or more strength enhancing polymeric constituents 340 may have a strain at fracture greater than 0.5, or even greater.

As the fluid 360 concentration in the structural framework or elements 304, 310 or segments increases, they may further transition from solid to semi-solid or viscoelastic. Thus, upon diffusion or penetration of physiological fluid 360 into the three-dimensional structural framework, or into one or more elements, or into one or more segments 304, 310, the drug-containing solid 301 (or the three-dimensional structural framework 304, or one or more elements 310, or one or more segments) may transition from solid to a semi-solid or viscoelastic mass 312.

Because the concentration of excipient in the semi-solid or viscoelastic mass 312 decreases as it absorbs water or physiological fluid, the stiffness of the semi-solid or viscous mass 312 generally decreases as it expands. In the invention herein, therefore, for ensuring that the stiffness and strength of the semi-solid or viscoelastic mass 312 remains so large that the (mechanical or geometric) integrity of the semi-solid or viscoelastic mass 312 is substantially preserved for prolonged time under the relevant physiological conditions, the normalized expansion of the drug-containing solid 301 (or of the framework 304 or of the semi-solid or viscoelastic mass 312) may be limited. More specifically, in some embodiments, a length, width, thickness, diameter, etc. of the drug-containing solid 301 (or of the framework of of the semi-solid or viscoelastic mass 312) may expand to no more than 5 times the initial value (e.g., the initial length of the drug-containing solid or framework prior to exposure to said physiological fluid) upon immersion in a physiological fluid. This includes, but is not limited to a length, width, thickness, diameter, etc. of the drug-containing solid (or of the framework or of the semi-solid or viscous mass) expanding to no more than 4.5 times, or no more than 4 times, or no more than 3.5 times, or no more than 3, or no more than 2.5 times the initial value prior to exposure to said physiological fluid.

Concomitant with the entrance or penetration of fluid 360 into the elements 310, drug molecules 320 may be released from the drug-containing solid 301 or semi-solid or viscoelastic mass 312 into the physiological fluid 360. By way of example but not by way of limitation, drug molecules may diffuse from the drug-containing solid 301 or semi-solid or viscoelastic dosage form 312 into the physiological fluid 360, FIGS. 3c-3f. If the amount of drug per unit volume of the semi-solid or viscoelastic mass is far greater than the solubility, and the semi-solid or viscoelastic mass 312 is several millimeters thick and stabilized or preserved for prolonged time, the drug release time can be prolonged.

As a result, upon immersion of the drug-containing solid or dosage form in a physiological fluid, said fluid may percolate at least an interconnected free space and diffuse into one or more elements (e.g., fibers), so that the framework expands in at least one dimension and transitions to a semi-solid mass. The expanded semi-solid mass may have a length between 1.3 and 4 times the initial length of the drug-containing solid prior to exposure to said physiological fluid. The semi-solid mass may further release drug over prolonged time (e.g., over a time greater than an hour, or over a time greater than two hours, or over a time greater than 5 hours, etc.).

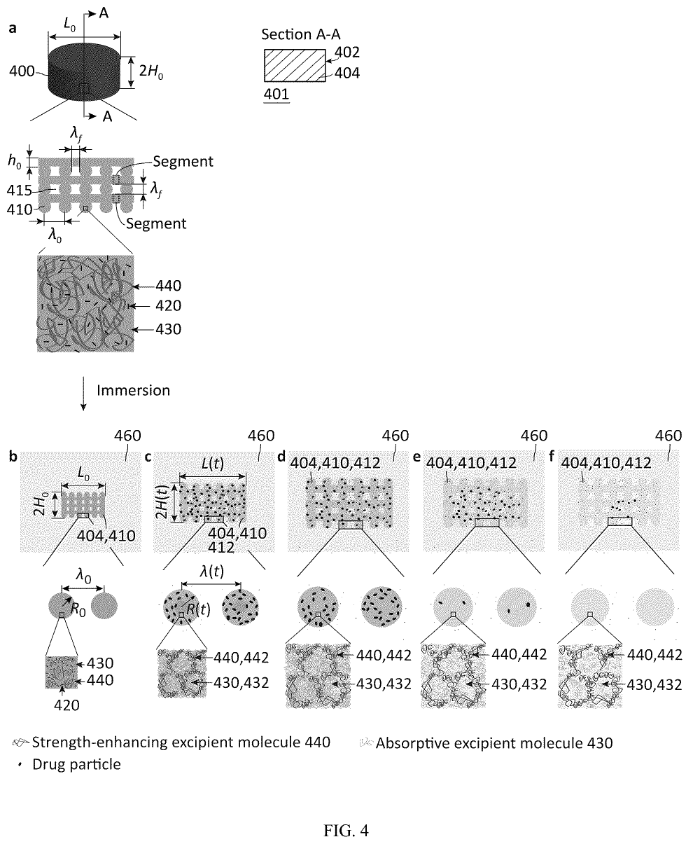

FIG. 4a schematically shows a further non-limiting example of a pharmaceutical dosage form disclosed herein. The dosage form 400 comprises a drug-containing solid 401 having an outer surface 402 and an internal three dimensional structural framework 404 of one or more thin structural elements 410. (It may be noted that in some preferred embodiments, the framework 404 comprises criss-crossed stacked layers of one or more fibers 410.) The framework 404 is contiguous with and terminates at said outer surface 402. The elements 410 have segments spaced apart from adjoining segments, thereby defining one or more interconnected free spaces 415 through the drug-containing solid 401. The elements 410 further comprise at least one active ingredient 420 and at least two excipients 430, 440. The at least two excipients 430, 440 comprise one or more physiological fluid-absorptive polymeric excipients or constituents 430 and one or more strength-enhancing polymeric excipients or constituents 440.

Upon exposure to a physiological fluid 460, said one or more fluid-absorptive excipients 430 transition to a viscous mass or a viscous solution 432, FIGS. 4b-4c. Said one or more fluid-absorptive excipients or viscous mass or viscous solution 430, 432 further expand said framework 404 along at least one dimension with absorption of said physiological fluid 460, FIGS. 4b-4f. Also, upon exposure to a physiological fluid 460, said one or more strength-enhancing excipients 440 form a fluid-permeable, semi-solid network 442 to mechanically support said framework 404, as illustrated schematically in the non-limiting FIGS. 4c-4f.

FIG. 4a also presents another non-limiting example of a pharmaceutical dosage form disclosed herein. The dosage form 400 comprises a drug-containing solid 401 having an outer surface 402 and an internal three dimensional structural framework 404 of one or more thin structural elements 410. (It may be noted that in some preferred embodiments, the framework 404 comprises criss-crossed stacked layers of one or more fibers 410.) The framework 404 is contiguous with and terminates at said outer surface 402. The elements 410 have segments spaced apart from adjoining segments, thereby defining one or more interconnected free spaces 415 through the drug-containing solid 401. The elements 410 further comprise at least one active ingredient 420 and at least two excipients 430, 440.

The at least two excipients 430, 440 comprise one or more fluid-absorptive polymeric constituents or excipients 430 within which 430 the solubility of a physiological fluid is greater than 600 mg/ml. The at least two excipients 430, 440 further comprise one or more strength-enhancing polymeric constituents or excipients 440. After soaking with said physiological fluid under physiological conditions, the one or more strength-enhancing polymeric constituents 440 have an elastic modulus in the range between 0.1 MPa and 200 MPa, and a strain at fracture greater than 0.5. Preferably, moreover, the one or more strength-enhancing polymeric constituents 440 form one or more phases that are substantially connected and/or substantially contiguous along the lengths of one or more structural elements 410.

As shown schematically in FIGS. 4b-4f, upon immersion in, or upon exposure to, said physiological fluid 460, said fluid 460 percolates at least one interconnected free space 415 and diffuses into one or more elements 410, thereby expanding said framework 404 in at least one dimension to a length between 1.3 and 4 times its length prior to exposure to said physiological fluid 460. By way of example but not by way of limitation, the initial thickness of said framework 404 (also referred to herein as the “thickness prior to exposure to said physiological fluid”), H₀, may expand to a thickness, H=1.3-4 times H₀, upon exposure of said framework 404 to said physiological fluid 460. Similarly, the initial length of said framework 404, L₀, may expand to a length, L=1.3-4 times L₀, upon exposure of said framework 404 to said physiological fluid 460.

Also, upon exposure to said physiological fluid 460, said framework 404 or drug-containing solid 401 transitions to a semi-solid mass 412. The semi-solid mass 412 may maintain its length between 1.3 and 4 times the initial length of said framework 404 or drug-containing solid 401 for prolonged time, FIGS. 4c-4f. The term “the semi-solid mass maintains its length between 1.3 and 4 times the initial length for prolonged time” is referred to herein as a semi-solid mass immersed in an unstirred or lightly stirred dissolution fluid (e.g., acidic water), wherein said immersed semi-solid mass maintains its length between 1.3 and 4 times the initial length for an extended time, such as a time greater than 1 hour, or a time greater than 2 hours, or a time greater than 5 hours, or an even longer time. Generally, also, said semi-solid mass 412 may release said drug 420 into said physiological fluid 460 over time (e.g., over a time greater than 1 hour, or over a time greater than 2 hours, or over a time greater than 5 hours, etc.).

Moreover, upon exposure to said physiological fluid, the one or more strength-enhancing excipients 440 form a fluid-permeable, semi-solid network 442 within one or more elements 410 or semi-solid mass 412 to mechanically support the one or more elements 410, framework 404, or semi-solid mass 412, FIGS. 4c-4f. Also, the one or more fluid-absorptive excipients 430 transition to a viscous mass, or a viscous solution 432, expanding said one or more elements 410 or framework 404 in at least one dimension with absorption of said physiological fluid 460.

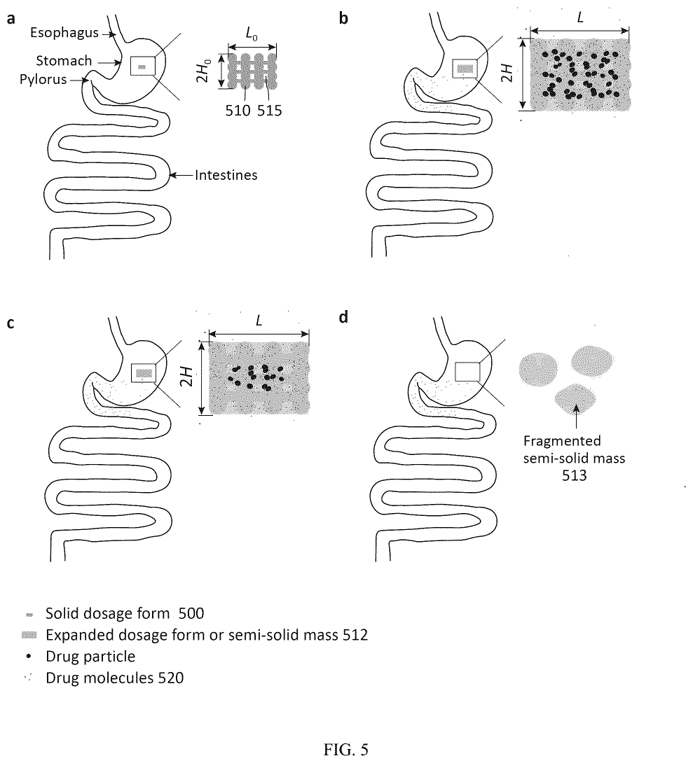

A non-limiting course of a dosage form structure after ingestion by a human or animal subject (e.g., a dog, a pig, etc.) is presented in FIG. 5. Initially, the dosage form 500 is solid and has a swallowable size and geometry. Upon ingestion, the dosage form enters the stomach, and interconnected free space 515 is percolated by gastric fluid (and/or by saliva, oesophageal fluid, etc.), FIG. 5a. The gastric fluid (and/or saliva, oesophageal fluid, etc.) then diffuses into the three dimensional structural framework and/or the elements 510 (e.g., fibers) it surrounds. As a result, the drug-containing solid expands and a semi-solid mass 512 is formed with a size (e.g., a width, diameter, etc.) greater than the diameter or width of the pylorus and a strength or stiffness so large that it is substantially unfragmentable in the gastric environment (e.g., under normal gastric conditions) for prolonged time, FIG. 5b.

Moreover, as the drug-containing solid absorbs gastric fluid and transitions to a semi-solid mass, drug molecules may be released from the drug-containing solid or the semi-solid mass into the gastric fluid over prolonged time, FIGS. 5b and 5c. Thus, because the size and the strength or stiffness of the semi-solid mass 512 may remain sufficiently large to prevent its passage through the pylorus into the intestines for prolonged time, drug release into the stomach can be prolonged and/or controlled. Eventually, however, the stiffness or strength of the semi-solid mass 512, 513 may be so low that it disintegrates, or deforms excessively, or breaks up, or fragments, or dissolves, etc. in the stomach, and passes into the intestines, FIG. 5d. It may be noted that the terms “disintegrate” or “disintegration” are used as equivalents to “fragment”, “fragmentation”, “deform”, “excessive deformation”, “dissolve”, “dissolution”, “erode”, “erosion”, “mechanically weaken”, “soften”, “break up”, “rupture”, and so on.

Additional aspects and embodiments of structural elements or fibers according to the invention herein are described throughout this specification. Any more aspects and embodiments of structural elements or fibers obvious to a person of ordinary skill in the art are all within the spirit and scope of this invention.

### Models of Expansion, Drug Release, and Disintegration of the Dosage Form

The following examples present non-limiting ways by which the expansion and drug release behavior of the disclosed dosage forms may be modeled. They will enable one of skill in the art to more readily understand the details and advantages of the invention. The models and examples are for illustrative purposes only, and are not meant to be limiting in any way.

### (a) Dosage Form Microstructures and Formulation

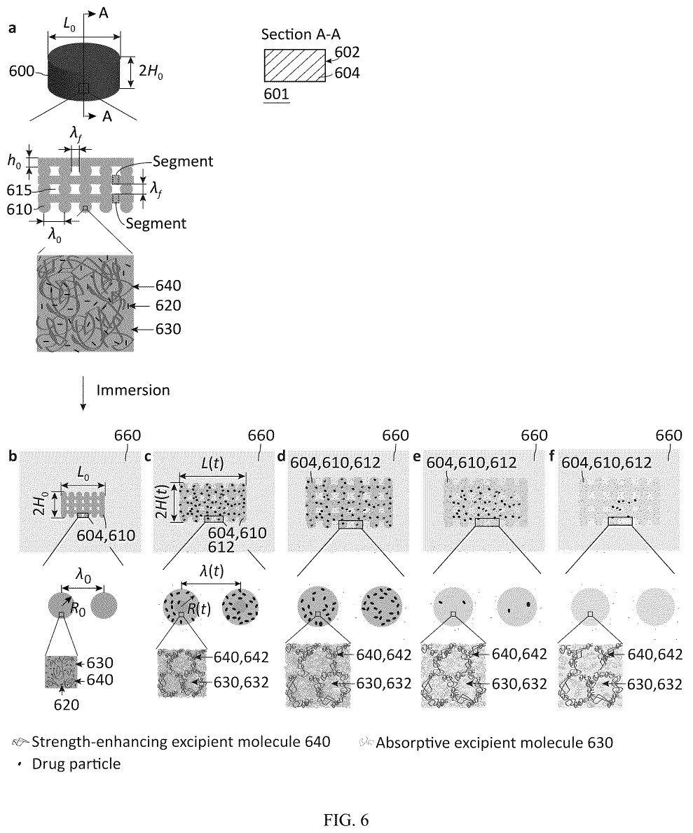

The non-limiting models refer to dosage forms as shown schematically in the non-limiting FIG. 6a. The dosage forms 600 comprise a drug-containing solid 601 having an outer surface 602 and an internal three dimensional structural framework 604 comprising a plurality of criss-crossed stacked layers of one or more fibrous elements 610, said framework contiguous with and terminating at (e.g., and defining) said outer surface 602. The fibrous elements 610 have segments spaced apart from like segments of adjoining elements, thereby defining free spaces 615, wherein a plurality of adjacent free spaces of successive layers combine to define one or more interconnected free spaces 615 through the drug-containing solid 601. At least one of said one or more interconnected free spaces 615 terminates at said outer surface 602 and is filled with matter removable by a physiological fluid under physiological conditions. The fibrous elements 610 further comprise at least one active ingredient 620 and at least two excipients 630, 640 through their thickness. The at least two excipients 630, 640 comprise one or more physiological fluid-absorptive polymeric constituents 630 of molecular weight greater than 50 kg/mol and one or more strength-enhancing constituents 640. Moreover, in the specific non-limiting dosage forms considered in the models, said at least two excipients 630, 640 form at least a solid solution. Also, one or more phases comprising the one or more strength-enhancing excipients 640 are substantially connected or contiguous along the lengths of one or more fibers or the structural framework. Similarly, one or more phases comprising the one or more strength-enhancing excipients 640 are substantially connected or contiguous through the thicknesses of one or more fibers or the structural framework.

Furthermore, in the specific non-limiting examples herein, the physiological fluid-absorptive polymeric excipient 630 generally comprises hydroxypropylmethylcellulose (HPMC) of molecular weight 120 kg/mol. HPMC is mutually soluble with typical physiological fluids. Thus, the solubility of a physiological fluid in said absorptive excipient (e.g., HPMC) can be about 1000 mg/ml, or greater than 750 mg/ml. The strength-enhancing excipient 640 generally comprises methacrylic acid-ethyl acrylate copolymer (also referred to herein as “Eudragit L100-55”). The mechanical properties of Eudragit L100-55 after exposure to a physiological fluid are presented in Experimental example 2.7 and Table 6 of this disclosure. Briefly, after soaking with a physiological fluid, said strength-enhancing excipient 640 (e.g., Eudragit L100-55) comprises an elastic modulus of about 5.7 MPa (e.g., between 0.2 MPa and 200 MPa), a tensile strength of about 1.8 MPa (e.g., between 0.2 MPa and 200 MPa), and a strain at fracture of about 3.5 (e.g., greater than 0.5, or between 0.5 and 20). Moreover, said strength-enhancing excipient 640 (e.g., Eudragit L100-55) is an enteric excipient that is sparingly soluble or practically insoluble in aqueous media with a pH value smaller than about 5.5, but dissolves in aqueous media with a pH value greater than about 5.5-6. The drug 620 in the non-limiting dosage forms modeled herein generally comprises ibuprofen.

### (b) Concept of Expansion, Drug Release, and Disintegration of the Dosage Forms

As shown schematically in the non-limiting FIG. 6b, upon immersion of the dosage form 600 or drug-containing solid 601 in a stirred dissolution fluid 660, such as deionized (DI) water with 0.1 M hydrochloric acid (HCl), said fluid may percolate at least one interconnected free space 615 and wet the structural framework 604, 610. This may allow the fluid 660 to diffuse into one or more said fibrous elements 610, and the framework 604, 610 to expand along all dimensions and to transition to a semi-solid or viscoelastic or viscous mass 650.

Without wishing to be bound to a particular theory, moreover, within the elements or fibers, the solubility of the acidic fluid may be high in absorptive excipient (e.g., HPMC, etc.), but low in the strength-enhancing excipient (e.g., Eudragit L100-55, etc.). Thus, as the water concentration in the fibers increases, the excipients may separate out into at least two phases: a highly viscous solution of water and absorptive excipient (e.g., within polyhedral cells, cavities, etc. of the fibrous elements) and a semi-solid network (e.g., semi-solid membranes, a semi-solid polyhedral network of membranes, a semi-solid framework, a semi-solid network of cell walls, a semi-solid network of fibers, etc. within the fibrous elements) of strength-enhancing excipient, FIG. 6c. Water molecules may readily pass through the cell walls, or membranes, of the strength-enhancing excipient (or semi-solid network) into the cells, but passage of absorptive excipient molecules out of the cells may be hindered. As a result, an internal pressure may develop in the cells due to the inward osmotic flux of water and the cells, fibers, and dosage form may expand. The concentration of absorptive excipient molecules and the internal pressure in the cells, however, may decrease as they expand. Eventually, therefore, expansion may cease and an expanded composite semi-solid or viscoelastic or viscous mass may be formed that may comprise a substantially stable (e.g., a substantially constant or unchanged) geometry for prolonged time.

Moreover, as dissolution fluid (water, etc.) enters the fibers, the fibers may supersaturate with drug and the drug molecules may aggregate as particles until the solubility is reached (FIGS. 6c and 6d). Also the remaining drug molecules may diffuse out from the semi-solid dosage form or semi-solid mass or viscous mass into the dissolution fluid. As drug molecules are released, the drug particles in the fibers may dissolve back until they may be depleted, FIGS. 6d-6e. If the amount of drug per unit volume of the semi-solid or viscous mass is far greater than the solubility, and/or the semi-solid or viscous mass is several millimeters thick, the drug release time can be prolonged.

It may be noted, furthermore, that water-soluble components, such as absorptive excipient, etc. may dissolve slowly from the semi-solid or viscous mass, and the semi-solid or viscous mass may disintegrate with time. The way by which the semi-solid or viscous mass disintegrates may, however, depend on the conditions it is exposed to. By way of example but not by way of limitation, in a lightly stirred dissolution fluid the semi-solid or viscous mass may not deform (e.g., shear) substantially, and it may also not break up. However, if the semi-solid or viscous mass is exposed to repeated compression or exposed to impact, etc., as it might be in the stomach of a human or animal subject, the semi-solid or viscous mass may deform somewhat due to the forces acting on it, and it may eventually break up or rupture.

### (c) Expansion of Single Fibers

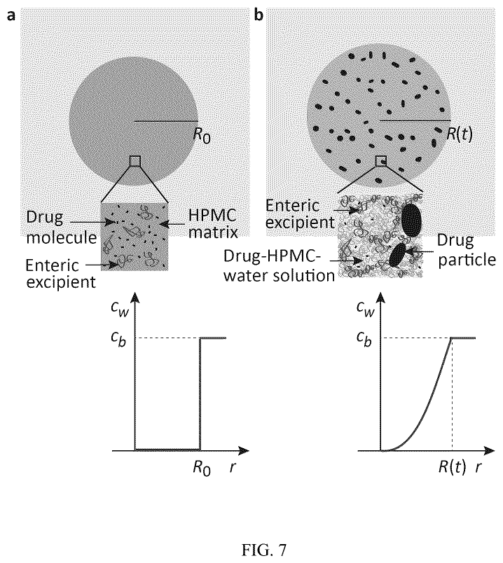

Upon immersion of a fiber in a dissolution fluid, the expansion rate of the fiber may be determined by the diffusive flux of water into the interior, as shown in the non-limiting FIG. 7. The governing diffusion equation in cylindrical coordinates may be written as:

$$\frac{\partial {c}_{w}}{\partial t} = \frac{1}{r} \frac{\partial }{\partial r} ( {rD}_{w} \frac{\partial {c}_{w}}{\partial r} ) 0 \leq r \leq R ( t ) \qquad ( 1 a )$$

where c_{w}(r,t) is the concentration of water in the fiber, D_{w} the diffusion coefficient of water in the fiber, and R(t) the fiber radius at time t.

Let the water concentration in the fiber at the fluid-fiber-interface be c_{b}. The initial and boundary conditions, as shown in FIG. 7, may then be written as:

c_{w}=0 t=0, 0≤r<R₀ (1b)

c_{w}=c_{b} t≥0, r=R(t) (1c)

where R₀ is the initial fiber radius, which increases as the mass of water in the fiber increases.

An analytical solution of Eq. (1a) subject to the initial condition (1b) and the moving-boundary condition (1c) may not be available at present. However, under the highly approximate assumptions that the diffusion coefficient of water through the fiber is constant and the concentration of water in the fiber is very small, the water concentration profile, as shown schematically in the non-limiting FIG. 7, may be approximated by (see, e.g., J. Crank, “The Mathematics of Diffusion”, second edition, Oxford University Press, 1975):

$$\frac{{c}_{w}}{{c}_{b}} = 1 - \frac{2}{R} {\sum }_{n = 1}^{∞} \frac{exp ( - {D}_{w} {α}_{n}^{2} t ) {J}_{0} ( r {α}_{n} )}{{α}_{n} {J}_{1} ( R {α}_{n} )} \qquad ( 2 )$$

where J₀ and J₁ are the Bessel functions of the first kind of order zero and one, respectively, and the αₙ's are the roots of

J₀(Rαₙ)=0 (3)

Integrating Eq. (2) over the fiber volume can give the ratio of the mass of water in the fiber per unit length at time t, M_{w}(t), and that at infinite time, M_{w,∞}. For small times (e.g., t<<R₀²/D_{w}),

$$\frac{{M}_{w} ( t )}{{M}_{w , ∞}} ≅ \frac{4}{\sqrt{π}} {( \frac{{D}_{w} t}{{R}_{0}^{2}} )}^{1 / 2} \qquad ( 4 )$$

The mass of water per unit length of the fiber may be written in terms of the water volume per unit length at time t, V_{w}(t), and the fiber volume per unit length as t→∞. Under the very approximate assumption that the fiber expansion is small,

M_{w}(t)=ρ_{w}V_{w}(t) (5a)

M_{w,∞}=c_{b}V₀ (5b)

where V₀ is the initial fiber volume per unit length.

From Eqs. (4) and (5) the normalized volumetric expansion of the fiber may be expressed as:

$$\frac{Δ V ( t )}{{V}_{0}} ≅ \frac{4}{\sqrt{π}} \frac{{c}_{b}}{{ρ}_{w}} {( \frac{{D}_{w} t}{{R}_{0}^{2}} )}^{1 / 2} \qquad ( 6 )$$

where ΔV(t)=V_{w}.

Further assuming that the fiber expands isotropically, the normalized radial and axial expansions may be about a third of the volumetric expansion. Thus, for small times and small expansions,

$$\frac{Δ R}{{R}_{0}} ≅ \frac{Δ L}{{L}_{0}} ≅ \frac{4}{3 \sqrt{π}} \frac{{c}_{b}}{{ρ}_{w}} {( \frac{{D}_{w} t}{{R}_{0}^{2}} )}^{1 / 2} \qquad ( 7 )$$

From Eq. (7) the rate at which the normalized radius and length of the fiber increases may increase if the boundary concentration and diffusivity of water are increased, and the fiber radius is decreased. Thus, for achieving rapid expansion, the diffusivity of water in the fibers or structural elements should be large, and the fiber radius (or element thickness) should be small.

For further information related to the diffusion of dissolution fluid into fibers or other geometries, see, e.g., J. Crank, “The Mathematics of Diffusion”, second edition, Oxford University Press, 1975. More models for estimating the expansion rate of the fibers obvious to a person of ordinary skill in the art are all within the spirit and scope of this disclosure.

### (d) Expansion of Dosage Forms

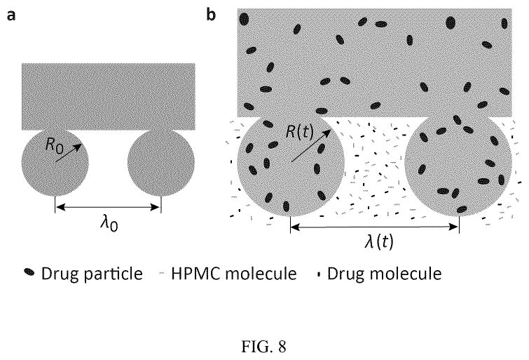

Upon immersion of a fibrous dosage form in a dissolution fluid, the dissolution fluid may percolate one or more free spaces and diffuse into one or more fibers. As a result, the one or more fibers may expand, as shown schematically in the non-limiting FIG. 8.

Because the dosage form may expand due to water diffusion into the fibers, the normalized longitudinal expansion of the dosage form, ΔL/L₀|_{DF}, may be related to normalized longitudinal and axial expansions of the single fiber, ΔL/L₀|_{SF} and ΔR/R₀|_{SF}, as:

$${{{\frac{Δ L}{{L}_{0}} \vert}_{DF} ≅ {k}_{LL} \frac{Δ L}{{L}_{0}} \vert}_{SF} ≅ {k}_{RL} \frac{Δ R}{{R}_{0}} \vert}_{SF} \qquad ( 8 )$$

where k_{LL} and k_{RL} are constants, and ΔL/L₀|_{SF} and ΔR/R₀|_{SF}, respectively, are the normalized longitudinal and radial expansions of the single fibers, Eq. (7). If the fibers expand isotropically, k_{LL} and k_{RL}˜1.

Thus, for some dosage forms where fiber expansion is isotropic, k_{LL} and k_{RL} are in the range of about 0.25 to 4 (this includes, but is not limited to a range of 0.5 to 2).

More models for estimating the dosage form's expansion rate that are obvious to a person of ordinary skill in the art are all within the spirit and scope of this disclosure.

### (e) Drug Release by the Dosage Forms

Because the drug in the non-limiting examples herein is sparingly soluble (e.g., the mass of drug in the expanded fiber per unit volume of the expanded fiber initially is greater than the drug solubility in the expanded fiber), as water diffuses into the fiber, the drug molecules in the fiber may precipitate as particles. The fiber may then be a “uniform” semi-solid or viscous mass of water, drug molecules, and drug particles. From this semi-solid or viscous fiber mass, drug molecules may diffuse out into the fluid-filled void or free space of the dosage form, and subsequently be transported into the dissolution fluid. Moreover, as the drug molecules diffuse out of the fibers, the drug particles in the fibers may dissolve back until they may be depleted.

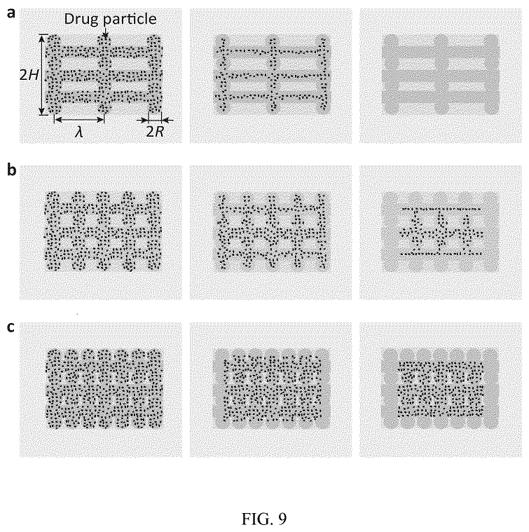

Three cases may be differentiated, FIG. 9. If the volume fraction of fibers in the dosage form is very small, as in FIG. 9a, the rate-determining diffusion length may be the radius of the thin, single fiber, and the drug release rate may be fast. If the fiber volume fraction is very large, however, as in FIG. 9c, the rate-determining diffusion length may be the half-thickness of the corresponding thick, monolithic slab, and the drug release rate may be very slow. If the fiber volume fraction is intermediate, as in FIG. 9b, the drug release rate may be between these two extremes. The two extreme cases are modeled below.

### (e1) Case 1: Drug Release Limited by Diffusion Through the Fiber (2R/λ˜0)

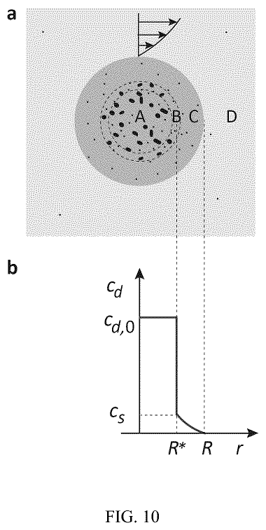

In the first case, the fibers are very far apart and the fluid velocity around the fibers is so large that the drug release rate by the fibrous dosage form is limited by the rate of diffusion within the fibers. In the fibers, two regions may be differentiated, as shown in the non-limiting FIG. 10: a drug particle-dispersed region and a drug particle-depleted region containing only dissolved drug molecules. The two regions may be delineated by a thin, inward-moving boundary with thickness of the order of the inter-particle distance.

In the particle-dispersed region, the total drug mass (drug particles plus drug molecules) per unit volume may be the initial value, and far greater than the drug solubility, FIG. 7b. In the particle-free region, the drug concentration may be governed by the diffusion equation:

$$\frac{\partial {c}_{d}}{\partial t} = \frac{1}{r} \frac{\partial }{\partial r} ( r {D}_{d} \frac{\partial {c}_{d}}{\partial r} ) {R}^{*} ( t ) \leq r \leq R \qquad ( 9 a )$$

subject to the initial, interfacial, and boundary conditions

c_{d}=c_{d,0} t=0, r≤R (9b)

c_{d}=cₛ t>0, r=R*(t) (9c)

c_{d}=0 r≥R (9d)

where D_{d} is the diffusivity of drug molecules, c_{d,0} is the “initial” drug mass (drug particles plus drug molecules) in the expanded fiber per unit volume of the expanded fiber, cₛ the drug solubility in the expanded fiber, R*(t) the radius of the particle-dispersed region, and R the radius of the expanded fiber.

Condition (9c) may stipulate that the mass of drug particles depleted from the moving boundary can be equal to the mass of drug that diffuses out as molecules. Thus, by mass conservation in a differential volume at the interface the following condition may be written:

(c_{d,0}−cₛ)ΔR*=D_{d}(dc_{d}/dr)Δt r=R*(t) (9e)

where ΔR* is the change in the radius of the interface in the time interval Δt. Rearranging and rewriting in differential form

$$\frac{d {R}^{*}}{d t} = \frac{{D}_{d}}{{c}_{d , 0} - {c}_{s}} \frac{{dc}_{d}}{dr} r = {R}^{*} ( t ) \qquad ( 9 f )$$

An analytical solution to Eq. (9a) subject to the conditions (9b) to (9f) may not be available at present. However, if c_{d,0}>>cₛ, as in the present, non-limiting case, the concentration profile in the particle-depleted region may be assumed quasi-steady (for further details related to the quasi-steady state, see, e.g., J. Crank, “The Mathematics of Diffusion”, second edition, Oxford University Press, 1975). That is, the drug concentration in the particle-free region may be expressed as:

$${c}_{d} = \frac{{c}_{s} ln ( r / R )}{ln ( {R}^{*} ( t ) / R )} {R}^{*} ( r ) \leq r \leq R \qquad ( 10 )$$

Differentiating, the concentration gradient at the interface way be written as:

$$\frac{d {c}_{d}}{dr} = \frac{{c}_{s}}{r ln ( {R}^{*} ( t ) / R )} r = {R}^{*} ( t ) \qquad ( 11 )$$

Combining Eq. (9f) and Eq. (11) can give the velocity of the interface as:

$$\frac{d {R}^{*}}{d t} = \frac{{D}_{d}}{{c}_{d , 0} - {c}_{s}} \frac{{c}_{s}}{{R}^{*} ( t ) ln ( {R}^{*} ( t ) / R )} \qquad ( 12 )$$

Rearranging and rewriting in integral form

$${\int }_{R}^{{R}^{*} ( t )} {R}^{*} ( t ) ln ( \frac{{R}^{*} ( t )}{R} ) {dR}^{*} = {\int }_{0}^{t} \frac{{D}_{d} {c}_{s}}{{c}_{d , 0} - {c}_{s}} dt \qquad ( 13 )$$

### Integrating,

$$\frac{1}{4} {{R}^{*} ( t )}^{2} ( 2 ln ( \frac{{R}^{*} ( t )}{R} ) - 1 ) + \frac{1}{4} {R}^{2} = \frac{{D}_{d} {c}_{s}}{{c}_{d , 0} - {c}_{s}} t \qquad ( 14 )$$

### Rewriting,

$${( \frac{{R}^{*} ( t )}{R} )}^{2} ( 1 - 2 ln ( \frac{{R}^{*} ( t )}{R} ) ) = 1 - \frac{4 {c}_{s}}{{c}_{d , 0} - {c}_{s}} \frac{{D}_{d} t}{{R}^{2}} \qquad ( 15 )$$

From geometry the fraction of drug released by the fiber in time t may be written as:

$$\frac{{m}_{d}}{{M}_{0}} = 1 - {( \frac{{R}^{*} ( t )}{R} )}^{2} \qquad ( 16 )$$

Combining Eqs. (15) and (16) gives an implicit equation for the fraction of drug released based on the relevant geometric and physico-chemical parameters:

$$( 1 - \frac{{m}_{d}}{{M}_{0}} ) ( 1 - ln ( 1 - \frac{{m}_{d}}{{M}_{0}} ) ) = 1 - \frac{4 {c}_{s}}{{c}_{d , 0} - {c}_{s}} \frac{{D}_{d} t}{{R}^{2}} \qquad ( 17 )$$

For small times, Eq. (17) can be simplified by expanding m_{d}/M₀ as a power series (e.g., substituting ln(1−m_{d}/M₀)=−m_{d}/M₀) as:

$$\frac{{m}_{d}}{{M}_{0}} = 2 {( \frac{{c}_{s}}{{c}_{d , 0} - {c}_{s}} )}^{1 / 2} {( \frac{{D}_{d} t}{{R}^{2}} )}^{1 / 2} \qquad ( 18 )$$

Moreover, substituting m_{d}/M₀=0.8 in Eq. (18) and rearranging, the time to release 80 percent of the drug content may be estimated by:

$${t}_{0.8} = 0.12 \frac{( {c}_{d , 0} - {c}_{s} ) {R}^{2}}{{c}_{s} {D}_{d}} \qquad ( 19 )$$

Thus, by Eq. (19) the drug release time may increase if the concentration of drug in the fiber divided by the solubility and the fiber radius are increased, and the diffusivity of drug through the fiber is decreased.

For further information related to the diffusion of drug out of fibers or other geometries, see, e.g., J. Crank, “The Mathematics of Diffusion”, second edition, Oxford University Press, 1975. More models for estimating the drug release rate and time by single fibers in a reasonably-well stirred dissolution fluid obvious to a person of ordinary skill in the art are all within the spirit and scope of this disclosure.

### (e2) Case 2: Drug Release Limited by Diffusion Through the Monolithic Semi-Solid Dosage Form (2R/λ˜1)

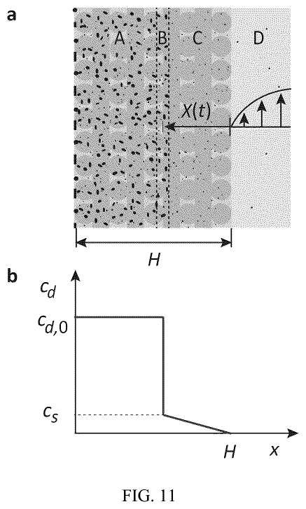

In the second case, the fibers are so tightly packed that the expanded semi-solid or viscous dosage form is essentially a monolithic slab, as illustrated in the non-limiting FIG. 10. If the dissolution fluid is stirred and the dosage form several millimeters thick, drug release is limited by diffusion through the slab. In analogy to the single-fiber diffusion, the particle-dispersed and particle-depleted regions are delineated by an inward-moving interface, FIG. 11. At the moving interface the mass of depleted drug particles may be the same as the mass of drug that diffuses out. Thus

(c_{d,0}−cₛ)ΔX=D_{d}(dc_{d}/dx)Δt x=H−X(t) (20)

where c_{d,0} is the initial drug mass per unit volume of the slab, H the half-thickness of the slab, X(t) the advancement of the interfacial position at time t, and ΔX the incremental advancement of the interfacial position in the time interval Δt. Rearranging and rewriting in differential form

$$\frac{d X}{d t} = \frac{{D}_{d}}{{c}_{d , 0} - {c}_{s}} \frac{\partial {c}_{d}}{\partial x} x = H - X ( t ) \qquad ( 21 )$$

According to the quasi-steady state assumption, now the concentration profile may be linear, FIG. 10b. Thus the velocity of the interface toward the origin, dX/dt, may be expressed as:

$$\frac{d X}{d t} ≅ \frac{{c}_{s}}{{c}_{d , 0}} \frac{{D}_{d}}{X} \qquad ( 22 )$$

Rearranging and integrating,

$$X = {( \frac{2 {c}_{s} {D}_{d} t}{{c}_{d , 0}} )}^{1 / 2} \qquad ( 23 )$$

The fraction of drug released, m_{d}/M₀, may be about equal to X/H, where H is the half-thickness of the semi-solid or viscous dosage form mass. Thus the fraction of drug released may be approximated by

$$\frac{{m}_{d}}{{M}_{0}} ≅ {( \frac{2 {c}_{s} {D}_{d} t}{{c}_{d , 0} {H}^{2}} )}^{1 / 2} \qquad ( 24 )$$

and the time to release eighty percent of the drug content,

$${t}_{0.8} = 0.32 \frac{( {c}_{d , 0} - {c}_{s} ) {H}^{2}}{{c}_{s} {D}_{d}} \qquad ( 25 )$$

From Eq. (25), t_{0.8} may be proportional to H².

More models for estimating the drug release rate and time by a monolithic slab would be obvious to a person of ordinary skill in the art. All are within the spirit and scope of this disclosure.

### (f) Disintegration of the Dosage Form

The rate of disintegration of the semi-solid or viscous dosage form generally depends greatly on the forces it is exposed to. Because a preferred application of the dosage form herein is prolonged drug release into the stomach, herein a highly approximate model for estimating the disintegration time of the dosage form in the stomach is developed.

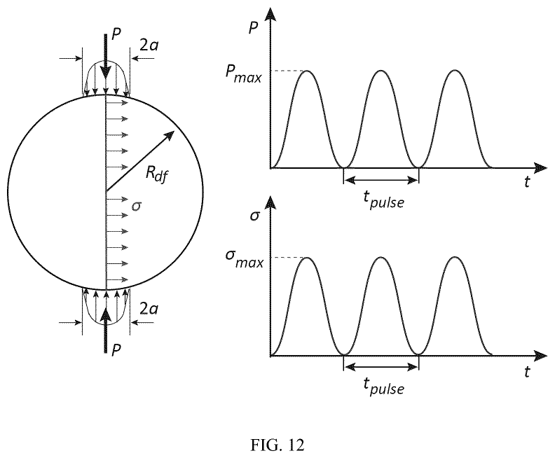

In the stomach, the dosage form generally is exposed to cyclic compressive forces by the stomach walls. A non-limiting force field acting on the expanded semi-solid or viscous dosage form comprises diametrically opposed cyclic loads per unit length, P, with maximum load per unit length, Pₘₐₓ, as shown schematically in FIG. 12. The corresponding maximum cyclic stress (tension) along the axis of symmetry may be approximated as (for further details, see, e.g., A. H. Blaesi and N. Saka, Int. J. Pharm. 509 (2016) 444-453):

$${σ}_{max} = \frac{{P}_{max}}{π {R}_{d f}} \qquad ( 26 )$$

where σₘₐₓ is the maximum cyclic tensile stress along the axis of symmetry of dosage form, Pₘₐₓ the maximum load intensity (load per unit length) applied by the stomach walls, and R_{df} the radius of the expanded dosage form.

To avoid immediate fracture of the dosage form, the tensile strength of the expanded, semi-solid or viscous dosage form should be greater than σₘₐₓ. If the tensile strength of the expanded, semi-solid or viscous dosage form is greater than σₘₐₓ, the dosage form may exhibit fatigue fracture after a number of compression pulses, N_{f}.

Assuming that Pₘₐₓ, σₘₐₓ, and the stiffness, strength, geometry, etc. of the dosage form are time-invariant, in analogy with Basquin's equation, a power function for the fatigue life, or number of compression pulses to failure, N_{f}, of the dosage form may be proposed as:

σₘₐₓ=σ_{f,df}N_{f}^{b} (27)

where σ_{f,df} is the tensile strength of the dosage form, and b is a constant, typically of the order −0.12.

Generally, the tensile strength of the expanded, semi-solid or viscous dosage form may predominantly be determined by the characteristics of the strength-enhancing excipient network. Under the highly approximate assumption that the strength-enhancing excipient network in or around the fibers or elements may be considered a cellular material, the tensile strength of the dosage form may be expressed as (for further details, see, e.g., M. F. Ashby, Metall. Trans. A 14A (1983) 1755-1769):

σ_{f,df}=σ_{f,se}C₈φₛₑ^{3/2} (28)

where σ_{f,se} is the fracture strength of the acidic water-soaked strength-enhancing excipient, φₛₑ its volume fraction in the dosage form, and C₈ a constant, typically about equal to 0.65.

Substituting Eq. (28) in Eq. (27) and rearranging gives:

$${N}_{f} = {( \frac{{σ}_{max}}{{σ}_{f , se} {C}_{8} {φ}_{se}^{3 / 2}} )}^{1 / b} \qquad ( 29 )$$

The gastric residence time, tᵣ˜N_{f}×tₚᵤₗₛₑ, where tₚᵤₗₛₑ is the period of a compression cycle by the stomach walls Substituting this term in Eq. (29) and rearranging gives:

$${t}_{r} ∼ {{t}_{pulse} ( \frac{{σ}_{max}}{{σ}_{f , se} {C}_{8} {φ}_{se}^{3 / 2}} )}^{1 / b} \qquad ( 30 )$$

where tₚᵤₗₛₑ is the period of a compression cycle by the stomach walls.

Combining Eq. (30) with Eq. (26) gives:

$${t}_{r} ∼ {{t}_{pulse} ( \frac{{P}_{max}}{π {R}_{df} {σ}_{f , se} {C}_{8} {φ}_{se}^{3 / 2}} )}^{1 / b} \qquad ( 31 )$$

By Eq. (31), the parameters that may be changed to alter the gastric residence time are the radius of the dosage form, R_{df}, the fracture strength of the acidic water-soaked excipient in monotonic loading, σ_{f,se}, and the volume fraction of the strength-enhancing excipient in the dosage form, σₛₑ. The radius of the dosage form, however, cannot be changed over a large range. Similarly, for the given formulation, σ_{f,se} is generally given. Thus the primary variable that may be adjusted to control the gastric residence time is σₛₑ. From the non-limiting experimental results shown later, for σₛₑ˜0.2-0.5 the gastric residence time of the fibrous dosage form may be prolonged to greater than about a day. Such gastric residence time is sufficient to prolong the delivery of drug into the upper gastrointestinal tract, and improve the efficacy, safety, and convenience of a myriad of drug therapies.

### Embodiments of the Dosage Form

In view of the theoretical models and non-limiting examples above, which are suggestive and approximate rather than exact, and other considerations, the dosage forms disclosed herein may further comprise the following embodiments.

### a) Outer Geometry of Drug-Containing Solid and Three Dimensional Structural Framework of Elements

In some embodiments, the average length, and/or the average width, and/or average thickness of the drug-containing solid (e.g., the three dimensional structural framework of one or more elements) is/are greater than 1 mm. This includes, but is not limited to an average length, and/or average width, and/or average thickness of the drug-containing solid greater than 1.5 mm, or greater than 2 mm, or greater than 3 mm, or in the ranges 1 mm-30 mm, 1.5 mm-30 mm, 2 mm-30 mm, 5 mm-20 mm, 5 mm-18 mm, 6 mm-20 mm, 7 mm-20 mm, 7 mm-19 mm, 7 mm-18 mm, 7 mm-17 mm, 7 mm-16 mm, 8 mm-20 mm, 8 mm-18 mm, 8 mm-16 mm, 8 mm-15 mm, 8 mm-14 mm, 8 mm-13 mm, 8 mm-12 mm. In the invention herein, the length is usually referred to a measure of distance in direction of the longest distance, the thickness is usually referred to a measure of distance in direction of the shortest distance, and the width is smaller than the length but greater than the thickness. Moreover, in some embodiments the direction of the “width” may be perpendicular to the direction of the length and/or to the direction of the thickness.

In some embodiments, moreover, a width perpendicular to the direction of the longest dimension of the dosage form or drug-containing solid herein is greater than 6 mm. This includes, but is not limited to a width perpendicular to the direction of the longest dimension of the dosage form or drug-containing solid greater than 7 mm, or greater than 8 mm, or greater than 9 mm, or in the ranges 6 mm-18 mm, 6 mm-16 mm, 6 mm-15 mm, 7 mm-18 mm, 7 mm-16 mm, 7 mm-15 mm, or 8 mm-18 mm, 8 mm-16 mm, or 8 mm-15 mm.

The dosage forms or drug-containing solids or three dimensional structural frameworks herein can have any common or uncommon outer shape of a drug-containing solid. For non-limiting examples of common tablet shapes, see, e.g., K. Alexander, Dosage forms and their routes of Administration, in M. Hacker, W. Messer, and K. Bachmann, Pharmacology: Principles and Practice, Academic Press, 2009. Any other geometries, outer shapes, or dimensions of dosage forms, drug-containing solids, or three dimensional structural frameworks of elements obvious to a person of ordinary skill in the art are all within the spirit and scope of this invention.

### b) Surface Composition of Elements and Segments

In some embodiments, for enabling rapid percolation of dissolution fluid into the interior of the dosage form structure (e.g., into interconnected free space of the drug-containing solid), the surface composition of at least one element is hydrophilic. Such embodiments include, but are not limited to embodiments where the surface composition of one or more structural elements and/or the surface composition of one or more segments and/or the surface composition of the three dimensional structural framework of elements is hydrophilic. In this disclosure, a surface or surface composition is hydrophilic, also referred to as “wettable by a physiological fluid”, if the contact angle of a droplet of physiological fluid on said surface in air is no more than 90 degrees. This includes, but is not limited to a contact angle of a droplet of said fluid on said solid surface in air no more than 80 degrees, or no more than 70 degrees, or no more than 60 degrees, or no more than 50 degrees, or no more than 40 degrees, or no more than 30 degrees. It may be noted that in some embodiments the contact angle may not be stationary. In this case, a solid surface may be understood “hydrophilic” if the contact angle of a droplet of physiological fluid on said solid surface in air is no more than 90 degrees (including but not limiting to no more than 80 degrees, or no more than 70 degrees, or no more than 60 degrees, or no more than 50 degrees, or no more than 40 degrees) at least 20-360 seconds after the droplet has been deposited on said surface. A non-limiting schematic of a droplet on a surface is presented in U.S. application Ser. No. 15/482,776 titled “Fibrous dosage form”.

Generally, the percolation rate of physiological fluid into interconnected free space is increased if the contact angle between said fluid and the surface of the three dimensional structural framework of one or more elements is decreased. Thus, in some embodiments, at least one element, or at least one segment of an element, or the three dimensional structural framework of elements comprises a hydrophilic or highly hydrophilic coating for enhancing the rate of fluid percolation into the dosage form structure. In the context herein, a solid surface (e.g., a solid material or a solid compound or a surface or a coating) is understood “highly hydrophilic” if the contact angle of a droplet of physiological fluid on the surface of said solid in air is no more is no more than 45 degrees. This includes, but is not limited to a contact angle of a droplet of said fluid on said solid surface in air no more than 35 degrees, or no more than 30 degrees, or no more than 25 degrees, or no more than 20 degrees, or no more than 15 degrees.

Non-limiting examples of hydrophilic (or highly hydrophilic) compounds that may serve as coating of elements (or segments of elements, or the three dimensional structural framework of elements) include polyethylene glycol, polyvinyl alcohol, polyvinyl alcohol-polyethylene glycol copolymer, polyvinyl pyrrolidone, silicon dioxide, sugars or polyols (e.g., mannitol, maltitol, xylitol, maltitol, isomalt, lactitol, sucrose, glucose, fructose, galactose, erythritol, maltodextrin, etc.), and so on.

In preferred embodiments, the coating of one or more elements comprises at least a polyol. In other preferred embodiments, the coating of one or more elements comprises at least a sugar, such as sucrose, fructose, glucose, or galactose. In other preferred embodiments, the coating of one or more elements comprises at least silicon dioxide.

Any other compositions or coatings of the surface of one or more elements or the three dimensional structural framework that would be obvious to a person of ordinary skill in the art are all included in this invention.

### c) Microstructure of Drug-Containing Solid and Three Dimensional Structural Framework

In some embodiments, dissolution fluid may percolate into the interior of the structure (e.g., into at least one free space or into the free spaces) if the drug-containing solid comprises at least a continuous channel or free space having at least two openings in contact with said fluid. The more such channels exist with at least two ends in contact with a dissolution fluid the more uniformly may the structure be percolated. Also, the greater the space over which a continuous channel having at least two ends in contact with a dissolution fluid extends, the more uniformly may the structure be percolated. Uniform percolation is desirable in the invention herein.

Thus, in the invention herein a plurality of adjacent free spaces may combine to define one or more interconnected free spaces (e.g., free spaces that are “contiguous” or “in direct contact” or “merged” or “without any wall in between”) forming an open pore network that extends over a length at least half the thickness of the drug-containing solid, or over a length greater than at least twice the thickness of one or more elements. This includes, but is not limited to a plurality of adjacent free spaces combining to define one or more interconnected free spaces forming an open pore network that extends over a length at least two thirds the thickness of the drug-containing solid, or over a length at least equal to the thickness of the drug-containing solid, or over a length at least equal to the side length of the drug-containing solid, or over a length and width at least equal to half the thickness of the drug-containing solid, or over a length and width at least equal to the thickness of the drug-containing solid, or over a length, width, and thickness at least equal to half the thickness of the drug-containing solid, or over a length, width, and thickness at least equal to two thirds the thickness of the drug-containing solid, or over a length, width, and thickness at least equal to the thickness of the drug-containing solid, or over the entire length, width, and thickness of the drug-containing solid.

Also, in some embodiments an open pore network comprises or occupies at least 30 percent (e.g., at least 40 percent, or at least 50 percent, or at least 60 percent, or at least 70 percent, or at least 80 percent, or 100 percent) of the free space of the drug-containing solid (e.g., at least 30 percent, or at least 40 percent, or at least 50 percent, or at least 60 percent, or at least 70 percent, or at least 80 percent, or at least 85 percent, or at least 90 percent, or at least 95 percent, or at least 98 percent, or 100 percent of the free space of the drug-containing solid are part of the same open pore network).

In preferred embodiments, all free spaces are interconnected forming a continuous, single open pore network. In the invention herein, if all free spaces of a drug-containing solid are interconnected the free space of said drug-containing solid is also referred to as “contiguous”. The elements or three dimensional structural framework may essentially form a three dimensional lattice structure surrounded by contiguous or interconnected free space. In preferred embodiments, moreover, one or more interconnected free spaces terminate at the outer surface of the drug-containing solid.

In drug-containing solids with contiguous free space that terminates at the outer surface of the drug-containing solid, no walls (e.g., walls comprising the three dimensional structural framework of elements) must be ruptured to obtain an interconnected cluster of free space (e.g., an open channel of free space) from the outer surface of the drug-containing solid (or from any point within the free space) to a point (or to any point) in the free space within the internal structure. The entire free space or essentially all free spaces is/are connected and accessible from (e.g., connected to) the outer surface of the drug-containing solid.

FIG. 13 schematically illustrates a pharmaceutical dosage form 1300 comprising a drug-containing solid 1301 having an outer surface 1302 and an internal three dimensional structural framework 1304 comprising a plurality of criss-crossed stacked layers of one or more fibrous elements 1310. Said framework 1304 is contiguous with and terminates at said outer surface 1102. The fibrous elements 1310 further have segments spaced apart from like segments of adjoining elements, thereby defining free spaces 1320. A plurality of adjacent free spaces 1325 combine to define one or more interconnected free spaces forming an open pore network 1330.

As shown in the non-limiting schematic of section A-A, free space 1320 is interconnected through the drug-containing solid 1301, and said open pore network 1330 extends over the entire length and thickness of the drug-containing solid 1301 or the dosage form 1300. In other words, the length, Lₚₒᵣₑ, over which the open pore network 1330 extends is the same as the length or diameter, D, of the dosage form 1300 or drug-containing solid 1301; the thickness, Hₚₒᵣₑ, over which the open pore network 1330 extends is the same as the thickness, H, of the dosage form 1300 or drug-containing solid 1301. It may be noted that the term “section” is understood herein as “plane” or “surface”. Thus a “section” is not a “projection” or “projected view”.

Moreover, in the non-limiting example of FIG. 13 the microstructure is rotationally symmetric. If the plane or section A-A is rotated by 90 degrees about the central axis the microstructure (e.g., the microstructural details) is/are the same. Thus, the open pore network 1330 also extends over the entire width of the drug-containing solid 1301 or the dosage form 1300. In other words, the width over which the open pore network 1330 extends is the same as the width or diameter, D, of the dosage form 1100 or drug-containing solid 1101.

Furthermore, in the non-limiting microstructure of FIG. 13, as shown in section A-A the open pore network 1330 or free space 1320 or free spaces 1325 is/are contiguous, and free space 1320 terminates at the outer surface 1302 of the drug-containing solid 1301. No walls (e.g., walls comprising the three dimensional structural framework 1304 of elements) must be ruptured to obtain an interconnected cluster of free spaces (e.g., an open channel of free space) from the outer surface 1302 of the drug-containing solid 1301 to a point (or to any point or position) in the free space 1320, 1325, 1330. The entire free space 1320, 1325, 1330 is accessible from the outer surface 1302 of the drug-containing solid 1301. Also, no walls (e.g., walls comprising the three dimensional structural framework 1304 of elements) must be ruptured to obtain an interconnected cluster of free space (e.g., an open channel of free space) from any point or position within the free space 1320, 1325, 1330 to any other point or position in the free space 1320, 1325, 1330. The entire free space 1320, 1325, 1330 is accessible from any point, location, or position within the free space 1320, 1325, 1330.

Additionally, the structure shown in FIG. 13 comprises fibers (or fiber segments) in a layer that are aligned unidirectionally (e.g., parallel). The fibers (or fiber segments) in the layers above and below said layer are aligned unidirectionally, too, and are aligned orthogonally to the fibers in said layer (e.g., the fibers in the the layers above and below said layer are aligned orthogonally to the fibers in said layer, and vice versa). The fibers in the layers above and below said layer further touch or “merge with” fibers in said layer at inter-fiber point contacts. Thus the structural framework, network, or 3D-lattice structure may be considered a network comprising nodes or vertices at the inter-fiber point contacts and edges defined by the fiber segments between adjacent nodes or vertices. In the specific example of FIG. 13 the distance, λ, of fiber segments between adjacent point contacts is uniform or constant across the network.

Therefore, in some embodiments, the three dimensional structural framework herein comprises a fibrous network having inter-fiber point contacts and fiber segments between adjacent contacts, and wherein the length of fiber segments between adjacent point contacts is uniform across the fibrous network. It may be noted that in some embodiments of the invention herein, a variable (e.g., a length, distance, width, angle, concentration, etc.) is uniform across the structural framework (e.g., across the fibrous network) if the standard deviation of multiple (e.g., multiple, randomly selected, e.g., at least three or at least 4 or at least 5 or at least 6 or at least 10 or at least 20 randomly selected) counts of said variable across the structural framework is less than the average value. This includes, but is not limited to a standard deviation of multiple (e.g., multiple, randomly selected, e.g., at least three or at least 4 or at least 5 or at least 6 or at least 10 or at least 20 randomly selected) counts of said variable across the structural framework less than half the average value, or less than one third of the average value, or less than a quarter of the average value, or less than one fifth of the average value, or less than one sixth of the average value, or less than one eight of the average value, or less than one tenth of the average value, or less than one fifteenth of the average value. The term “uniform” is also referred to herein as “constant” or “almost constant” or “about constant”.

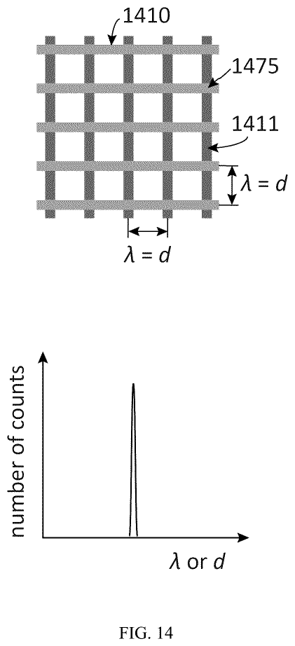

The graph of FIG. 14 is a histogram of the length, λ, of fiber segments between adjacent point contacts. The λ values in this non-limiting example are distributed in a very narrow window or zone around the average, λ_{avg}. Thus the standard deviation of the λ values is very small; λ is precisely controlled; the structure is regular, deterministic, and ordered.

In some embodiments, therefore, the three dimensional structural network herein comprises a fibrous network having inter-fiber point contacts 1475 and fiber segments 1410, 1411 between adjacent contacts, and wherein the length of fiber segments between adjacent point contacts is precisely controlled. It may be noted that the dosage form properties (e.g., the uniformity of fluid percolation into the drug-containing solid, the expansion rate, the drug release rate, etc.) can be optimized if the microstructural parameters are precisely controlled. In the invention herein, the term “precisely controlled” is also referred to as “ordered” or “orderly arranged”. A variable or a parameter (e.g., the spacing of fiber segments between point contacts, the contact width, the fiber thickness, the spacing between fibers, etc.) is precisely controlled if it is deterministic and not stochastic (or random). A variable or parameter may be deterministic if, upon multiple repetitions of a step that includes said variable (e.g., if multiple dosage forms are produced under identical or almost identical conditions), the standard deviation of the values of said variable is smaller than the average value. This includes, but is not limited to a standard deviation of the values of said variable smaller than half the average value, or smaller than one third of the average value, or smaller than a quarter of the average value, or smaller than one fifth or the average value, or smaller than one sixth, or smaller than one seventh, or smaller than one eight, or smaller than one ninth, or smaller than one tenth, or smaller than 1/12, or smaller than 1/15, or smaller than 1/20, or smaller than 1/25 of the average value of said variable, or smaller than 1/30 of the average value of said variable.

Moreover, in some embodiments, the three dimensional structural network or framework herein comprises a fibrous network having inter-fiber point contacts and fiber segments between adjacent contacts, and wherein the average length of fiber segments between adjacent point contacts is between 1 and 15 times the average thickness of the one or more fibers. This includes, but is not limited to fibrous networks having inter-fiber point contacts and fiber segments between adjacent contacts, and wherein the average length of fiber segments between adjacent point contacts is between 1 and 12 times, or between 1 and 10 times, or between 1 and 9 times, or between 1 and 8 times, or between 1 and 7 times, or between 1 and 6 times, or between 1 and 5 times, or between 1 and 4.5 times, or between 1 and 4 times the average thickness of the one or more fibers.

More generally, in some embodiments, the volume fraction of elements (e.g., fibers) in the drug-containing solid (e.g., the element (e.g., fiber) volume divided by the volume of the drug-containing solid) is in the range of 0.1 to 0.95. This includes, but is not limited to a volume fraction of elements in the drug-containing solid in the ranges 0.15-0.95, 0.15-0.9, 0.15-0.85, 0.2-0.95, 0.2-0.9, 0.2-0.85, 0.25-0.95, 0.25-0.9, or 0.25-0.85.

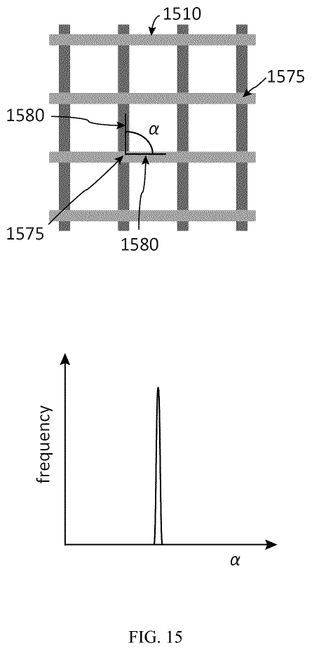

As shown in the structure of FIG. 15, moreover, at the inter-fiber point contacts 1575 the two tangents of two contacting fibers or fiber segments 1580, may form an angle, α. If the distance, λ, of fiber segments between point contacts is uniform or constant across the network, the angle, α, formed by two tangents of contacting fiber segments (e.g., the angle of intersection) at the contact is about 90°. It may be noted, however, that the angle formed by two tangents of contacting fiber segments (e.g., the angle of intersection) can also assume other values, including without limitation an average value greater than 0 degrees. This includes, but is not limited to an angle formed by two tangents of contacting fiber segments (e.g., the angle of intersection) in the ranges between 20 and 90 degrees, or 30-90, or 40-90, or 50-90, or 60-90, or 70-90 degrees. Furthermore, also the angle formed by two tangents of contacting fiber segments (e.g., the angle of intersection) can be precisely controlled. Thus, in some embodiments herein, the three-dimensional structural network of fibers comprises a fibrous network having inter-fiber point contacts defined by intersecting fibers or fiber segments, and wherein the angle of intersection at said point contacts is precisely controlled across said fibrous network.

More examples of fibrous structures according to the invention herein would be obvious to a person of ordinary skill in the art. All of them are within the scope of this disclosure.

It may further be noted, however, that in some embodiments the three dimensional structural framework comprises stacked layers (or plies) of particles, fibers, or sheets, or any combinations thereof. In some embodiments, moreover, one or more layers or plies are bonded to the layers above or below said one or more layers.

Furthermore, many of the above features and characteristics may also apply to (e.g., the features or characteristics may be similar to the features or characteristics of) three-dimensional structural frameworks of stacked layers of sheets, or beads (e.g., particles) shown, for example, in the co-pending International Application No. PCT/US2019/052030 filed on Sep. 19, 2019, and titled “Dosage form comprising structured solid-solution framework of sparingly-soluble drug and method for manufacture thereof”. Such features or characteristics are obvious to a person of ordinary skill in the art who is given all information disclosed in this specification. Application of such features or characteristics to three-dimensional structural frameworks of stacked layers of beads (e.g., particles) or even sheets (e.g., two-dimensional elements), or any combinations of fibers, beads, and/or sheets, is included in the invention herein.

Further non-limiting embodiments of the dosage form structure are presented in U.S. application Ser. No. 15/482,776 titled “Fibrous dosage form”, U.S. application Ser. No. 15/964,058 titled “Method and apparatus for the manufacture of fibrous dosage forms”, the U.S. application Ser. No. 15/964,063 and titled “Dosage form comprising two-dimensional structural elements”, and the International Application No. PCT/US19/19004 titled “Expanding structured dosage form”. More examples of how the elements may be structured or arranged in the three dimensional structural framework of one or more solid elements would be obvious to a person of ordinary skill in the art. All of them are within the spirit and scope of this invention.

### (g) Inter-Element or Inter-Fiber Contact and Bonding

Because the individual elements (e.g., fibers, beads, sheets, etc.) are generally thin and slender they may bend or deform due to the application of mechanical load. Thus, in some embodiments, to provide mechanical support to the structure the three dimensional structural framework of elements may comprise contacts between elements or segments. Such inter-element contacts include, but are not limited to point contacts or line contacts.

In the invention herein, a point contact is referred to as having a contact area or contact zone (e.g., the common surface of the two elements or segments in contact) that extends over a length and width no greater than 2.5 mm. This includes, but is not limited to a contact width between two elements (or two segments) no greater than 2 mm, or no greater than 1.75 mm, or no greater than 1.5 mm. In other examples without limitation, a contact width, 2a, between two elements (or two segments) at a point contact may be no greater than 1.1 times the thickness of the contacting elements (or segments) at the position of the contact. This includes, but is not limited to a contact width, 2a, between two elements (or two segments) no greater than 1 time, or no greater 0.8 times, or no greater than 0.6 times the thickness of the contacting fibers (or segments) at the position of the contact. A line contact is referred to as having a contact area or contact zone that extends over a contact length far greater than the contact width. The contact width is typically no greater than 2.5 mm. Moreover, at the contact (e.g., at the contact zone of a point contact or at the contact zone of a line contact), elements or segments may be deformed. The geometry of said elements or segments at or near the contact (e.g., at or near a point contact or at or near a line contact) then is different form the geometry elsewhere. In some embodiments, at the contact an element is “flat” or “flattened”.

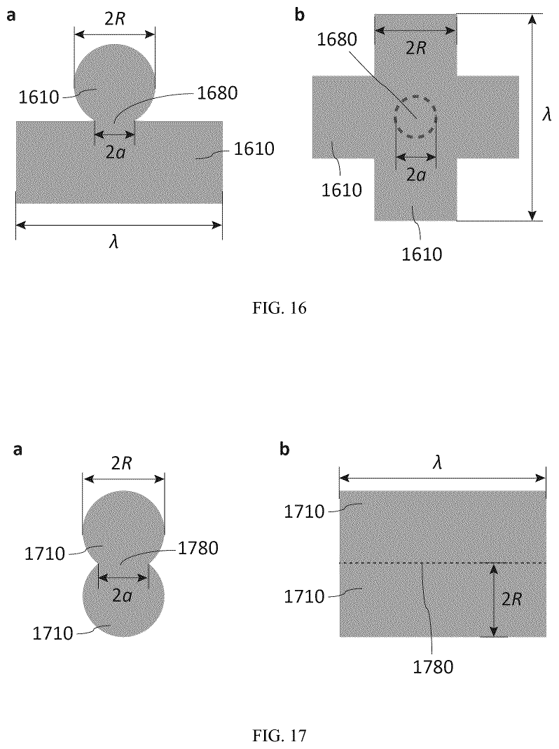

FIG. 16 is a non-limiting example of a point contact 1680 between two orthogonally aligned fiber segments 1610. FIG. 16a is the front view and FIG. 16b the top view of the two segments. The contact area is circular. The diameter of the circle or “contact width”, 2a, is designated in the Figure. FIG. 17 is a non-limiting example of a line contact 1780 between two unidirectionally aligned fiber segments 1710. FIG. 17a is the front view and FIG. 17b the top view. As shown in the Figure the contact width, 2a, is much smaller than the contact length, λ. Generally, because point contacts can enable better connectivity of free space, they may be preferred in some embodiments herein. For further information related to point contacts and line contacts, see, e.g., K. L. Johnson, “Contact mechanics”, Cambridge University Press, 1985.

In some embodiments, the number of point contacts in the three dimensional structural network is at least 10. This includes, but is not limited to a number of point contacts in the three dimensional structural network at least 20, or at least 50, or at least 75, or at least 100, or at least 125, or at least 150, or at least 175, or at least 200, or at least 250, or at least 300. In some embodiments, moreover, the number of point contacts in the three dimensional structural network is precisely controlled. In some embodiments, moreover, the number of line contacts in the three dimensional structural network is at least 10. In some embodiments, however, the number of line contacts in the three dimensional structural network is no greater than 10. In some embodiments, moreover, the number of line contacts in the three dimensional structural network is precisely controlled.

At the contact zone (e.g., at one or more point contacts or at one or more line contacts, etc.) two elements or segments may be bonded, which is understood herein as “fixed”, “joined”, “attached”, “welded” (e.g., by interdiffusion of molecules at the contact, such as interdiffusion of absorptive excipient from one element or segment to another contacting element or segment or interdiffusion of strength-enhancing excipient from one element or segment to another contacting element or segment, etc.), etc. Generally, the bond strength is a fraction of the bulk strength of the contacting elements or segments. Said fraction is typically no greater than 1. This includes, but is not limited to a bond strength no greater than 0.8, or no greater than 0.6, or no greater than 0.4 times the strength of the bulk of elements or segments. For providing mechanical support to the dosage form structure, however, the bond strength should generally be greater than 0.01, or greater than 0.02, or greater than 0.05, or greater than 0.1, or greater than 0.2, or greater than 0.3, or greater than 0.4, or greater than 0.5 times the bulk strength of elements or segments. In some embodiments, moreover, the bond strength is in the ranges 0.001-1, 0.01-1, 0.02-1, 0.05-1, 0.1-1, 0.2-1, 0.3-1, 0.4-1, 0.5-1, 0.001-0.95, 0.001-0.9, 0.005-1, 0.005-0.95, or 0.01-0.9 times the strength of the bulk of elements or segments. For further information about determining and measuring strength of solid materials, see, e.g., J. M Gere, S. Timoshenko, “Mechanics of materials”, fourth edition, PWS Publishing Company, 1997; M. F. Ashby, “Materials selection in mechanical design”, fourth edition, Butterworth-Heinemann, 2011; K. L. Johnson, “Contact mechanics”, Cambridge University Press, 1985.

Thus, in some embodiments, the three dimensional structural framework is a solid forming a continuous structure wherein at least one element (e.g., at least one fiber, etc.) or at least one segment of an element is bonded to another element or another segment. This includes, but is not limited to a three dimensional structural framework of elements forming a continuous solid structure wherein at least two elements or at least two segments, or at least three elements or at least three segments, or at least four elements or at least four segments, or at least five elements or at least five segments, are bonded to another element or another segment of an element.

Furthermore, the inter-element contacts may provide adequate or improved mechanical support to the three dimensional structural framework of elements, or to a semi-solid or viscous mass formed after immersion of said framework in a dissolution fluid, if the contact width between elements or segments is large enough. In some embodiments, therefore a contact width, 2a, between two elements (or two segments) is greater than 1 μm. This includes, but is not limited to a contact width between two elements or two segments greater than 2 μm, or greater than 5 μm, or greater than 10 μm. Moreover, in some embodiments, the average contact width between elements or segments across the three dimensional structural framework of elements is greater than 0.02 times the average thickness of said elements. This includes, but is not limited to average contact width between elements or segments across the three dimensional structural framework of elements greater than 0.05, or greater than 0.1, or greater than 0.2, or greater than 0.3, or greater than 0.4, or greater than 0.5 times the average thickness of elements or segments across the three dimensional structural framework. Moreover, in some embodiments average contact width between elements (or segments) across the three dimensional structural framework is in the ranges 1 μm-1 mm, 1 μm-2 mm, 2 μm-2 mm, 2 μm-1 mm, 5 μm-1.5 mm, 5 μm-1 mm, 10 μm-1.5 mm, 10 μm-1 mm, 15 μm-1 mm, 20 μm-1 mm, or 25 μm-1 mm.

It may be noted, moreover, that in some embodiments, the contact width of contacts between elements or segments in a dosage form or drug-containing solid or three dimensional structural framework of elements is precisely controlled. In some embodiments, furthermore, the number of contacts between elements (e.g., fibers, fiber segments, beads, sheets, etc.) or segments in a dosage form or drug-containing solid or three dimensional structural network is precisely controlled.

Any other features or characteristics of inter-element contacts or bonds obvious to a person of ordinary skill in the art are all included in this invention.

### d) Free Spacing Between Fibers or Elements

Typically, moreover, for dissolution fluid to percolate into the interior of the structure the channel size or diameter (e.g., channel width, or pore size, or free spacing, or effective free spacing) must be on the micro- or macro-scale. Thus, in some embodiments, the effective free spacing, λ_{f,e}, (or average effective free spacing) between elements or segments across one or more free spaces (e.g., interconnected free spaces through the drug-containing solid, or the pore size or pore diameter) is greater than 1 μm. This includes, but is not limited to λ_{f,e} (or average effective free spacing) greater than 1.25 μm, or greater than 1.5 μm, or greater than 1.75 μm, or greater than 2 μm, or greater than 5 μm, or greater than 7 μm, or greater than 10 μm, or greater than 15 μm, or greater than 20 μm, or greater than 25 μm, or greater than 30 μm, or greater than 40 μm, or greater than 50 μm.

Because the dosage form volume is generally limited, however, the drug and excipient masses that can be loaded in the dosage form may be too small if the effective free spacing is too large. Moreover, the free spacing between elements (and the volume fraction of elements) should not be too small to assure that the strength or viscosity of the semi-solid or viscous mass formed after immersion in a physiological fluid is sufficiently large. For these and/or other reasons, in some embodiments, the effective free spacing (or average effective free spacing) across an free space (e.g., an interconnected free space through the drug-containing solid or an open pore network) may be in the ranges 1 μm-5 mm, 1 μm-3 mm, 1 μm-2 mm, 1 μm-1.5 mm, 2 μm-4 mm, 2 μm-3 mm, 2 μm-2 mm, 5 μm-2.5 mm, 5 μm-2 mm, 5 μm-1.5 mm, 10 μm-2 mm, 10 μm-1.5 mm, 10 μm-3 mm, 15 μm-3 mm, 15 μm-1.5 mm, 20 μm-3 mm, 30 μm-3 mm, 40 μm-3 mm, or 40 μm-2 mm.

In some embodiments, moreover, the average effective free spacing between segments or elements across the one or more free spaces (e.g., across all free spaces of the dosage form) is in the range 1 μm-3 mm. This includes, but is not limited to an average effective free spacing between segments or elements across the one or more free spaces in the ranges 1 μm-2.5 mm, or 1 μm-2 mm, or 2 μm-3 mm, or 2 μm-2.5 mm, or 5 μm-3 mm, or 5 μm-2.5 mm, or 10 μm-3 mm, or 10 μm-2.5 mm, or 15 μm-3 mm, or 15 μm-2.5 mm, or 20 μm-3 mm, or 20 μm-2.5 mm. The effective free spacing may be determined experimentally from microstructural images (e.g., scanning electron micrographs, micro computed tomography scans, and so on) of the drug-containing solid. Non-limiting examples describing and illustrating how an effective free spacing may be determined from microstructural images are described and illustrated in the U.S. application Ser. No. 15/482,776 titled “Fibrous dosage form”.

It may be noted, moreover, that in some embodiments herein the free spacing or effective free spacing between elements or segments across the drug-containing solid, or across one or more interconnected free spaces, or across one or more open pore networks is precisely controlled.

Furthermore, the free spacing between elements and the surface composition of elements are generally designed to enable percolation of physiological, body, or dissolution fluid into the dosage form structure upon immersion of the dosage form in said fluid. Thus, in some embodiments the free spacing between segments and the composition of the surface of the one or more elements are so that the percolation time of physiological/body fluid into one or more free spaces (e.g., one or more interconnected free spaces) of the drug-containing solid is no greater than 30 minutes under physiological conditions.

In addition, in some embodiments, upon immersion of the drug-containing solid in a physiological fluid, said fluid percolates more than 20 or 40 percent of the free spaces of said drug-containing solid in no more than 600 seconds of immersion.

It should be obvious to a person of ordinary skill in the art that the free spaces, free spacings, or effective free spacings herein may comprise many more dimensions, characteristics, and features. All of them are included in this disclosure and invention.

### (e) Composition of Free Space

Generally, one or more free spaces (e.g., one or more interconnected free spaces) are filled with a matter that is removable by a physiological fluid under physiological conditions. Such matter that is removable by a physiological fluid under physiological conditions can, for example, be a gas which escapes the free space upon percolation by said physiological fluid. Such matter that is removable by a physiological fluid under physiological conditions can, however, also be a solid that is highly soluble in said physiological fluid, and thus dissolves rapidly upon contact with or immersion in said physiological fluid.

Non-limiting examples of biocompatible gases that may fill free space include air, nitrogen, CO₂, argon, oxygen, and nitric oxide, among others.

Non-limiting examples of solids that are removed or dissolved after contact with physiological/body fluid include sugars or polyols, such as Sucrose, Fructose, Galactose, Lactose, Maltose, Glucose, Maltodextrin, Mannitol, Maltitol, Isomalt, Lactitol, Xylitol, Sorbitol, among others. Other examples of solids include polymers, such as polyethylene glycol, polyvinyl pyrrolidone, polyvinyl alcohol, among others. Typically, a solid material should have a solubility in physiological/body fluid (e.g., an aqueous physiological or body fluid) under physiological conditions greater than 50 g/l to be removed or dissolved rapidly after contact with dissolution medium. This includes, but is not limited to a solubility greater than 75 g/l, or greater than 100 g/l, or greater than 150 g/l, or greater than 200 g/l. The diffusivity of the solid material (as dissolved molecule in physiological/body fluid under physiological conditions) should typically be greater than 4×10⁻¹² m²/s if the solid material must be dissolved rapidly after contact with dissolution medium. This includes, but is not limited to a diffusivity in physiological fluid under physiological conditions greater than 6×10⁻¹² m²/S or greater than 8×10⁻¹² m²/s, or greater than 1×10⁻¹¹ m²/s, or greater than 2×10⁻¹¹ m²/s, or greater than 5×10⁻¹¹ m²/s.

In some embodiments, moreover, a solid that may fill free space has a molecular weight (e.g., average molecular weight, such as number average molecular weight or weight average molecular weight) no greater than 80 kg/mol. This includes, but is not limited to a molecular weight (e.g., average molecular weight, such as number average molecular weight or weight average molecular weight) no greater than 70 kg/mol, or no greater than 60 kg/mol, or no greater than 50 kg/mol, or no greater than 45 kg/mol, or no greater than 40 kg/mol, or no greater than 35 kg/mol, or no greater than 30 kg/mol.

Further compositions of free space obvious to a person of ordinary skill in the art who is given all information of this specification are all included in this invention.

### (f) Element or Fiber Geometry

After percolation of free space or one or more interconnected free spaces, dissolution fluid or physiological fluid may surround one or more elements or segments (e.g., fibers, fiber segments, etc.). For achieving a large specific surface area (i.e., a large surface area-to-volume ratio) of solid in contact with dissolution fluid, in some embodiments the one or more elements (e.g., fibers, etc.) have an average thickness, h₀, no greater than 2.5 mm. This includes, but is not limited to h₀ no greater than 2 mm, or no greater than 1.75 mm, or no greater than 1.5 mm, or no greater than 1.25 mm, or no greater than 1 mm, or no greater than 750 μm, or no greater than 700 μm, or no greater than 650 μm, or no greater than 600 μm, or no greater than 550 μm, or no greater than 500 μm, or no greater than 450 μm.

It may be noted, however, that if the elements are very thin and tightly packed, the spacing and free spacing between the elements can be so small that the rate at which dissolution fluid percolates or flows into the free space is limited. Furthermore, dosage forms with very thin elements may be difficult to manufacture by, for example, 3D-micro-patterning or 3D-printing. Thus, in some embodiments the one or more elements have an average thickness, h₀, greater than 1 μm, or greater than 2 μm, or greater than 5 μm, or greater than 10 μm, or greater than 20 μm, or in the ranges of 5 μm-2 mm, 5 μm-1.5 mm, 5 μm-1.25 mm, 5 μm-1 mm, 5 μm-750 m, 5 μm-500 μm, 10 μm-2 mm, 10 μm-1.5 mm, 10 μm-1.25 mm, 10 μm-1 mm, 15 μm-1 mm, 20 μm-1 mm, 25 μm-1 mm, 30 μm-1 mm, 20 μm-1.5 mm, 25 μm-1.5 mm, 25 μm-1.25 mm, 25 μm-1 mm, 30 μm-1.5 mm, 30 μm-1.25 mm, 30 μm-1 μm, 40 μm-1.5 mm, or 40 μm-1 mm.

In some embodiments, moreover, the average thickness of the one or more elements (e.g., fibers, etc.) comprising (e.g., producing, making up, etc.) the three dimensional structural network (e.g., the average thickness of the elements (e.g., fibers, etc.) in the three dimensional structural network) is precisely controlled. Moreover, for ensuring constraint-free expansion, in some embodiments the thickness of one or more elements (e.g., wetted or wettable elements, fibers, wetted or wettable fibers, etc.) is uniform across said one or more elements. This includes, but is not limited to the thickness of elements uniform across the three dimensional structural framework of elements or the thickness of elements uniform across the drug-containing solid.

The element thickness, h, may be considered the smallest dimension of an element (i.e., h≤w and h≤l, where h, w and l are the thickness, width and length of the element, respectively). The average element thickness, h₀, is the average of the element thickness along the length and/or width of the one or more elements. A non-limiting example illustrating how the average element thickness may be derived is presented in U.S. application Ser. No. 15/482,776 titled “Fibrous dosage form”.

Generally, moreover, one or more elements (e.g., fibers, etc.) or segments (e.g., fiber segments, etc.) may comprise a continuous (e.g., a single, or internally connected) solid matrix through their thickness. In other words, the elements may comprise an outer element surface and an internal, continuous solid matrix that is contiguous with, terminating at, and/or defining said outer element surface.

In some embodiments, furthermore, at least one outer surface of an element (e.g., the outer surface or one or more fibers or the outer surface of a fiber segment) comprises a coating. Said coating may cover part of or the entire outer surface of one or more elements or segments. Said coating may further have a composition that is different or distinct from the composition of one or more elements or a segment. The coating may be a solid, and may or may not comprise or contain a drug.

### (f) Micro- and Nano-Structure and Composition of Drug-Containing Elements or Fibers

In the invention herein, the at least two excipients may have complementary functions or functionalities that may be required or necessary for producing an expandable, gastroretentive dosage form as disclosed herein. The micro- or nanostructure of the elements greatly affects their properties.

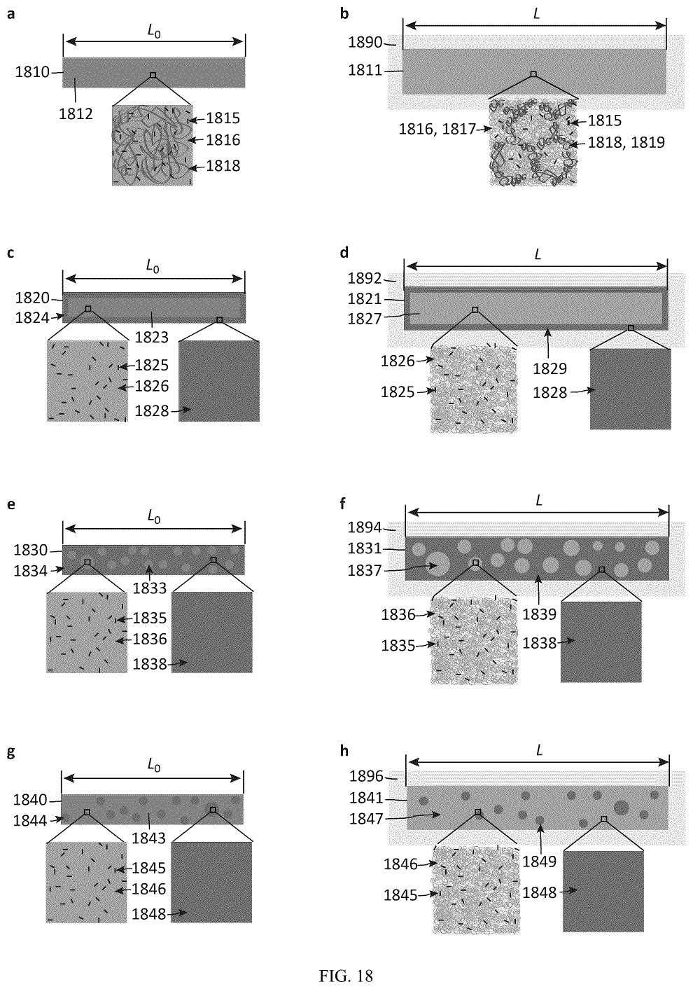

FIG. 18a presents a non-limiting example of an element 1810 (e.g., a fiber) comprising a solid solution 1812 of drug 1815, one or more physiological fluid-absorptive excipients 1816, and one or more strength-enhancing excipients 1818. The solid solution 1812 is a phase comprising strength-enhancing excipient 1818; it 1812 is connected along the length, L₀, of the element 1810. Thus, a phase 1812 comprising strength enhancing excipient 1818 is connected along the length of the element 1810.

Upon exposure to physiological fluid 1890, such as saliva, gastric fluid, a fluid that resembles a physiological fluid, and so on, the one or more strength-enhancing excipients 1818 form a fluid-permeable, semi-solid network 1819 to mechanically support the element 1811, FIG. 18b. Also, the one or more fluid-absorptive excipients 1816 transition to a viscous mass, or a viscous solution 1817, expanding said element 1810, 1811 along at least one dimension (or in all dimensions) with absorption of said physiological fluid 1890.

Because, a phase 1812 comprising strength enhancing excipient 1818 is connected along the length of the element 1810 prior to exposure to said physiological fluid 1890, a semi-solid network 1819 of strength-enhancing excipient 1818 is connected along the length, L, of the expanded element 1811. The connected, semi-solid network 1819 of strength-enhancing excipient 1818 mechanically supports or enforces the expanded element 1811.

FIG. 18c presents a non-limiting example of an element 1820 (e.g., a fiber) comprising a core 1823 of drug 1825 and absorptive excipient 1826 (without any dissolved molecules of strength-enhancing excipient), said core 1823 is surrounded by a layer or shell 1824 of strength-enhancing excipient 1828. The layer or shell 1824 of strength-enhancing excipient 1828 is connected along the length, L₀, of the element 1820. Thus, a phase 1824 comprising strength enhancing excipient 1828 is connected along the length of the element 1820.

Upon exposure to physiological fluid 1892, such as saliva, gastric fluid, a fluid that resembles a physiological fluid, and so on, the one or more strength-enhancing excipients 1828 form a fluid-permeable, semi-solid network 1829 to mechanically support the element 1821, FIG. 18d. Also, the one or more fluid-absorptive excipients 1826 transition to a viscous mass, or a viscous solution 1827, expanding said element 1820, 1821 along at least one dimension (or in all dimensions) with absorption of said physiological fluid 1892.

Because, a phase 1824 comprising strength enhancing excipient 1828 is connected along the length of the element 1820 prior to exposure to said physiological fluid 1892, a semi-solid network 1829 of strength-enhancing excipient 1828 is connected along the length, L, of the expanded element 1821. The connected, semi-solid network 1829 of strength-enhancing excipient 1828 mechanically supports or enforces the expanded element 1821.

FIG. 18e presents a non-limiting example of an element 1830 (e.g., a fiber) comprising dispersed particles 1833 of drug 1835 and absorptive excipient 1836 (without any dissolved molecules of strength-enhancing excipient) in a matrix 1834 of strength-enhancing excipient 1838. The matrix 1834 of strength-enhancing excipient 1838 is connected along the length of the element 1830. Thus, a phase 1834 comprising strength enhancing excipient 1838 is connected along the length of the element 1830.

Upon exposure to physiological fluid 1894, such as saliva, gastric fluid, a fluid that resembles a physiological fluid, and so on, the one or more strength-enhancing excipients 1838 form a fluid-permeable, semi-solid network 1839 to mechanically support the element 1831, FIG. 18f. Also, the one or more fluid-absorptive excipients 1836 transition to a viscous mass, or a viscous solution 1837, expanding said element 1830, 1831 along at least one dimension (or in all dimensions) with absorption of said physiological fluid 1894.

Because, a phase 1834 comprising strength enhancing excipient 1838 is connected along the length of the element 1830 prior to exposure to said physiological fluid 1894, a semi-solid network 1839 of strength-enhancing excipient 1838 is connected along the length, L, of the expanded element 1831. The connected, semi-solid network 1839 of strength-enhancing excipient 1838 mechanically supports or enforces the expanded element 1831.

FIG. 18g presents a non-limiting example of an element 1840 (e.g., a fiber) comprising a matrix 1843 of drug 1845 and absorptive excipient 1846 (without any dissolved molecules of strength-enhancing excipient), and dispersed particles 1844 of strength-enhancing excipient 1848. The dispersed particles 1844 of strength-enhancing excipient 1848 are not connected along the length, L₀, of the element 1840. Thus, the element or fiber 1840 does not include a phase comprising strength enhancing excipient 1848 that is connected along the length of the element 1840.

Upon exposure to physiological fluid 1896, such as saliva, gastric fluid, a fluid that resembles a physiological fluid, and so on, the one or more fluid-absorptive excipients 1846 transition to a viscous mass, or a viscous solution 1847, expanding said element 1840, 1841 along at least one dimension (or in all dimensions) with absorption of said physiological fluid 1896, FIG. 18h.

Because, a phase 1844 comprising strength enhancing excipient 1848 is not connected along the length of the element 1840 prior to exposure to said physiological fluid 1896, however, a semi-solid network of strength-enhancing excipient 1848 may not form along the length, L, of the expanded element 1841. (The strength-enhancing excipient 1848 may comprise dispersed particles 1849 in the expanded element 1841.) A semi-solid network of strength-enhancing excipient 1848 may not mechanically support or enforce the expanded element 1841 substantially. Such embodiments are generally not preferred in the invention herein. In some embodiments, therefore, one or more phases comprising strength-enhancing excipient form a connected or continuous or contiguous (or substantially connected or substantially continuous or substantially contiguous) network or structure or matrix within one or more elements or within the three dimensional structural framework of elements. In some embodiments, moreover, one or more phases comprising strength-enhancing excipient are substantially connected or substantially contiguous along the lengths of one or more structural elements or through the three dimensional structural framework.

It may be noted that generally, a phase or one or more phases comprising strength enhancing excipient is/are substantially connected along the length of one or ore elements or through a structural framework if the mechanical strength or stiffness (e.g., the elastic modulus) of said elements or framework after exposure to a physiological fluid is substantially greater than the mechanical strength or stiffness of an element or framework comprising fluid-absorptive excipient alone (e.g., no strength-enhancing excipient) after exposure to said physiological fluid. By way of example but not by way of limitation, one or more phases comprising strength enhancing excipient are connected along the length of an element if the tensile strength or the elastic modulus of said element after exposure to a physiological fluid is at least two times greater than that of a corresponding element comprising fluid-absorptive excipient alone (e.g., no strength-enhancing excipient) after exposure to said physiological fluid. This includes, but is not limited to the tensile strength or the elastic modulus of an element after exposure to a physiological fluid at least three times greater, or at least four times greater, or at least five times greater, or at least six times greater, or at least seven times greater than that of a corresponding element comprising fluid-absorptive excipient alone (e.g., no strength-enhancing excipient) after exposure to said physiological fluid.

In some embodiments, moreover, one or more phases comprising strength-enhancing excipient form a single continuous (e.g., a connected) structure or a single continuous (e.g., a connected) network structure along or through the elements of the three dimensional structural framework.

In some embodiments, moreover, the concentration of at least a strength-enhancing excipient is substantially uniform within or through or across one or more elements or the three dimensional structural framework of elements.

In some embodiments, the concentration of at least an absorptive excipient is substantially uniform within or through or across one or more elements or the three dimensional structural framework of elements.

In some embodiments, moreover, one or more elements comprise a plurality of (e.g., two or more) segments having substantially the same weight fraction of physiological fluid-absorptive excipient distributed within the segments (e.g., the standard deviation of the weight fraction of absorptive excipient within the elements or segments is no greater than the average value).

In some embodiments, moreover, one or more elements comprise a plurality of (e.g., two or more) segments having substantially the same weight fraction of strength-enhancing excipient distributed within the segments (e.g., the standard deviation of the weight fraction of strength-enhancing excipient within the elements or segments is no greater than the average value).

In some embodiments, moreover, the at least two excipients (e.g., at least an absorptive excipient and at least a strength-enhancing excipient) form a solid solution.

The properties of the combined at least two excipients together may further depend on the weight fractions of the individual constituents. More specifically, by altering the weight fractions of absorptive and strength-enhancing excipient in the three dimensional structural framework, relevant properties, such as expansion rate, extent of expansion, disintegration rate of the three dimensional structural framework, dissolution rate of the drug, etc. may be altered, adjusted, or controlled.

In some embodiments the weight fraction of absorptive polymeric excipient in at least one element with respect to the total weight of said element is greater than 0.1. This includes, but is not limited to a weight fraction of absorptive polymeric excipient in an element with respect to the total weight of said element greater than 0.15, or greater than 0.2, or greater than 0.25, or greater than 0.3, or greater than 0.35, or greater than 0.4.

Similarly, in some embodiments the weight fraction of absorptive polymeric excipient in the three dimensional structural framework of one or more elements with respect to the total weight of said framework is greater than 0.1. This includes, but is not limited to a weight fraction of absorptive, polymeric excipient in the structural framework with respect to the total weight of said framework greater than 0.15, or greater than 0.2, or greater than 0.25, or greater than 0.3, or greater than 0.35, or greater than 0.4.

In some embodiments, moreover, the weight fraction of absorptive polymeric excipient in at least one element with respect to the total weight of absorptive excipient and strength-enhancing excipient in said element is greater than 0.3. This includes, but is not limited to a weight fraction of absorptive polymeric excipient in an element with respect to the total weight of absorptive excipient and viscosity-enhancing excipient in said element greater than 0.4, or greater than 0.5, or greater than 0.6, or greater than 0.65, or greater than 0.7.

Similarly, in some embodiments the weight fraction of absorptive polymeric excipient in the three dimensional structural framework of one or more elements with respect to the total weight of absorptive excipient and strength-enhancing excipient in said framework is greater than 0.1. This includes, but is not limited to a weight fraction of absorptive, polymeric excipient in the structural framework with respect to the total weight of absorptive excipient and strength-enhancing excipient in said framework greater than 0.2, or greater than 0.3, or greater than 0.4, or greater than 0.5, or greater than 0.55.

In some embodiments, the weight fraction of strength-enhancing excipient with respect to the total weight of functional excipient (e.g., strength-enhancing excipient and absorptive excipient) is no greater than 0.9. This includes, but is not limited to a weight fraction of strength-enhancing excipient with respect to the total weight of functional excipient no greater than 0.85, or no greater than 0.8, or no greater than 0.75, or no greater than 0.7, or in the ranges 0.1-0.9, 0.1-0.85, 0.15-0.85, 0.15-0.9, 0.2-0.85, 0.2-0.9, 0.25-0.9, 0.25-0.85, 0.3-0.9, 0.3-0.85, 0.15-0.8, or 0.15-0.7.

In some embodiments, the volume of strength-enhancing excipient per unit volume of the dosage form or of a drug-containing solid (e.g., the volume fraction of strength-enhancing excipient in the dosage form or in a drug-containing solid with respect to the volume of said dosage form or of said drug-containing solid) is greater than 0.05. This includes, but is not limited to a volume of strength-enhancing excipient per unit volume of the dosage form or of a drug-containing solid (e.g., the volume fraction of strength-enhancing excipient in the dosage form or in a drug-containing solid with respect to the volume of said dosage form or of said drug-containing solid) greater than 0.1, or greater than 0.15, or greater than 0.2, or greater than 0.25.

In some embodiments, the weight of strength-enhancing excipient per unit volume of the dosage form or of a drug-containing solid (e.g., the density of strength-enhancing excipient in the dosage form or in a drug-containing solid with respect to the volume of said dosage form or of said drug-containing solid) is greater than 50 kg/m³. This includes, but is not limited to a weight of strength-enhancing excipient per unit volume of the dosage form or of a drug-containing solid (e.g., the density of strength-enhancing excipient in the dosage form or in a drug-containing solid with respect to the volume of said dosage form or of said drug-containing solid) greater than 100 kg/m³, or greater than 150 kg/m³, or greater than 200 kg/m3.

Any further microstructures of elements would be obvious to a person of ordinary skill in the art. All of them are within the spirit and scope of this invention.

### (f) Properties and Composition of Absorptive Excipient

The drug-containing elements herein comprise at least one ore more physiological fluid-absorptive excipients. In some specific embodiments embodiments, an absorptive excipient may be mutually soluble with a relevant physiological fluid under physiological conditions, and thus “absorb” or “mix with” said physiological fluid until its concentration is uniform across said fluid. Accordingly, absorptive excipient may promote expansion and dissolution and/or disintegration of a drug-containing solid or a semi-solid or viscous mass.

In some embodiments, moreover the effective diffusivity of physiological/body fluid in an absorptive excipient (and/or an element or a segment) is greater than 0.05×10⁻¹¹ m²/s under physiological conditions. This includes, but is not limited to an effective diffusivity of physiological/body fluid in an absorptive excipient (and/or an element or a segment) greater than 0.1×10⁻¹¹ m²/s or greater than 0.2×10⁻¹¹ m²/s, or greater than 0.5×10⁻¹¹ m²/s or greater than 0.75×10⁻¹¹ m²/s or greater than 1×10⁻¹¹ m²/s, or greater than 2×10⁻¹¹ m²/s or greater than 3×10⁻¹¹ m²/s or greater than 4×10⁻¹¹ m²/s under physiological conditions.

Alternatively, for absorptive excipients where diffusion of physiological/body fluid to the interior may or may not be Fickian, a rate of penetration may be specified. In some embodiments, the rate of penetration of a physiological/body fluid into a solid, absorptive excipient (and/or an element or a segment) is greater than an average thickness of the one or more drug-containing elements divided by 3600 seconds (i.e., h₀/3600 μm/s). In other examples without limitation, rate of penetration may be greater than h₀/1800 μm/s, greater than h₀/1200 μm/s, greater than h₀/800 μm/s, greater than h₀/600 μm/s, or greater than h₀/500 μm/s, or greater than h₀/400 μm/s, or greater than h₀/300 μm/s.

For determining the effective diffusivity (and/or the rate of penetration) of dissolution medium in a solid, absorptive excipient (and/or an element or a segment) the following procedure may be applied. An element (e.g an element or segment of the dosage form structure, or preferably an element or segment that just consists of the absorptive excipient) may be placed in a still dissolution medium at 37° C. The time t₁ for the element to break apart or deform substantially may be recorded. (By way of example but not by way of limitation, a deformation of an element may generally be considered substantial if either the length, width, or thickness of the element differs by at least 20 to 80 percent (e.g., at least 20 percent, or at least 30 percent, or at least 40 percent, or at least 50 percent, or at least 60 percent, or at least 70 percent, or at least 80 percent, etc.) from its initial value.) The effective diffusivity, D_{eff}, may then be determined according to D_{eff}=hᵢₙᵢₜ²/4t₁ where hᵢₙᵢₜ is the initial element or segment thickness (e.g., the thickness of the dry element or segment). Similarly, the rate of penetration of a physiological/body fluid into the element or segment may be equal to hᵢₙᵢₜ/2t₁. Further non-limiting examples for deriving the effective diffusivity or rate of penetration are presented in U.S. application Ser. No. 15/482,776 titled “Fibrous dosage form”.

To ensure that the drug-containing solid expands substantially, and that the integrity of the expanded semi-solid mass is preserved for prolonged time within a physiological fluid under physiological conditions, the molecular weight of the one or more physiological fluid-absorptive excipients should be quite large. In some embodiments, therefore, the molecular weight of at least one absorptive polymeric excipient is greater than 30 kg/mol. This includes, but is not limited to a molecular weight of an absorptive polymeric excipient greater than 40 kg/mol, or greater than 50 kg/mol, or greater than 60 kg/mol, or greater than 70 kg/mol, or greater than 80 kg/mol.

To ensure that the dosage form can be processed by patterning a viscous drug-excipient paste, and for other reasons, the molecular weight of at least one absorptive excipient (or the absorptive polymeric excipient in its totality) may be limited.

By way of example but not by way of limitation, the molecular weight of at least one absorptive excipient (or the average molecular weight of the absorptive excipient in its totality) may be in the ranges 30 kg/mol-10,000,000 kg/mol, 50 kg/mol-10,000,000 kg/mol, 70 kg/mol-10,000,000 kg/mol, 80 kg/mol-10,000,000 kg/mol, 70 kg/mol-5,000,000 kg/mol, 70 kg/mol-2,000,000 kg/mol. Preferably, a physiological fluid-absorptive excipient comprises hydroxypropyl methylcellulose with a molecular weight in the range between about 50 kg/mol and 500 kg/mol (e.g., 70 kg/mol-300,000 kg/mol).

Thus, in some embodiments, at least one absorptive excipient (or the absorptive excipient in its totality) may comprise a plurality of individual chains or molecules that dissolve or disentangle upon immersion in a physiological fluid.

In some embodiments, moreover, at least one absorptive excipient has a solubility greater than 20 g/l in a relevant physiological/body fluid under physiological conditions. This includes, but is not limited to at least one absorptive excipient (or the absorptive excipient in its totality) having a solubility in a relevant physiological/body fluid under physiological conditions greater than 50 g/l, or greater than 75 g/l, or greater than 100 g/l, or greater than 150 g/l, or greater than 175 g/l, or greater than 200 g/l, or greater than 250 g/l, or greater than 300 g/l, or greater than 350 g/l. In the extreme case, absorptive excipient (e.g., at least one absorptive excipient or the absorptive excipient in its totality) is mutually soluble with a relevant physiological fluid under physiological conditions. The solubility of a material is referred to herein as the maximum amount or mass of said material that can be dissolved at equilibrium in a given volume of physiological fluid under physiological conditions divided by the volume of said fluid or of the solution formed. By way of example but not by way of limitation, the solubility of a solute in a solvent may be determined by optical methods.

Preferably, moreover, at least one absorptive polymeric excipient (or the absorptive polymeric excipient in its totality) comprises an amorphous molecular structure (e.g., an amorphous arrangement of molecules, or an arrangement of molecules without long-range order) in the solid state. A non-limiting method for determining the molecular structure of a solid (e.g., distinguishing amorphous molecular structure from crystalline molecular structure, etc.) is Differential scanning calorimetry.

Non-limiting examples of excipients that satisfy some or all the requirements of an absorptive polymeric excipient include but are not limited to hydroxypropyl methylcellulose, hydroxyethyl cellulose, polyvinyl alcohol, polyvinylpyrrolidone, hydroxypropyl methylcellulose acetate succinate, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), vinylpyrrolidone-vinyl acetate copolymer, among others.

### (f) Properties and Composition of Strength-Enhancing Excipient

The drug-containing elements herein further comprise at least one ore more strength-enhancing excipients. Generally, at least one strength-enhancing excipient (or the strength-enhancing excipient in its totality), too, may be somewhat permeable to a relevant physiological fluid under physiological conditions to promote rapid expansion of the dosage form or drug-containing solid or framework upon immersion. In some embodiments, therefore, the diffusivity of a relevant physiological fluid under physiological conditions in at least one strength-enhancing excipient (or in the strength-enhancing excipient in its totality) is greater than 1×10⁻¹³ m²/s. This includes, but is not limited to a diffusivity of a relevant physiological fluid under physiological conditions in at least one strength-enhancing excipient (or in the strength-enhancing excipient in its totality) greater than 2×10⁻¹³ m²/s, or greater than 5×10⁻¹³ m²/s, or greater than 7×10⁻¹³ m²/s, or greater than 1×10⁻¹² m²/s, or greater than 2×10⁻¹² m²/s, or greater than 3×10⁻¹² m²/s, or greater than 4×10⁻¹² m²/s, or greater than 5×10⁻¹² m²/s, or greater than 6×10⁻¹² m²/s.

In some embodiments, moreover, upon immersion of an element, the three dimensional structural framework, or the dosage form in a relevant physiological fluid under physiological conditions, strength-enhancing excipient reduces or decreases or slows down the rate at which physiological fluid-absorptive excipient is removed, eroded, or dissolved from said element, or said three dimensional structural framework, or said the dosage form or semi-solid mass. By way of example but not by way of limitation, in some embodiments, upon immersion of an element (e.g., a fiber, etc.) in a relevant physiological fluid under physiological conditions, due to the presence of strength-enhancing excipient at a relevant quantity in said element, the rate at which physiological fluid-absorptive excipient is removed, eroded, or dissolved from said element can be substantially limited by the rate of diffusion of said absorptive excipient through said element.

In some embodiments, accordingly, upon immersion of an element in a relevant physiological fluid under physiological conditions, the diffusivity of at least one physiological fluid-absorptive excipient in or through said element is no greater than 5×10⁻¹² m²/s. This includes, but is not limited to a diffusivity of at least one physiological fluid-absorptive excipient in or through an element no greater than 2×10⁻¹² m²/s, or no greater than 1×10⁻¹² m²/s, or no greater than 5×10⁻¹³ m²/s, or no greater than 2×10⁻¹³ m²/s, or no greater than 1×10⁻¹³ m²/s, or no greater than 5×10⁻¹⁴ m²/s, or no greater than 2×10⁻¹⁴ m²/s.

In some embodiments, furthermore, upon immersion of an element in a relevant physiological fluid under physiological conditions, the diffusivity of at least one physiological fluid-absorptive excipient through an element (e.g., through a semi-solid element, or through a physiological fluid-penetrated element) is no greater than 0.3 times the self-diffusivity of said at least one absorptive excipient in a relevant physiological fluid under physiological conditions. This includes, but is not limited to the diffusivity of at least one absorptive excipient through an element (e.g., through a viscous element, or through a water-penetrated element) no greater than 0.2 times, or no greater than 0.1 times, or no greater than 0.05 times, or no greater than 0.02 times, or no greater than 0.01 times, or no greater than 0.005 times, or no greater than 0.002 times, or no greater than 0.001 times the self-diffusivity of said at least one absorptive excipient in a relevant physiological fluid under physiological conditions.

Generally, to assure that a strength-enhancing excipient remains a semi-solid or viscoelastic material and stabilizes, or mechanically supports or enforces one or more elements after exposure to a physiological fluid (e.g., gastric fluid, etc.), the solubility of said physiological fluid in said strength-enhancing excipient may be limited. In some embodiments, therefore, at least one strength-enhancing excipient has a solubility no greater than 1 g/l in a relevant physiological/body fluid under physiological conditions. This includes, but is not limited to at least one strength-enhancing excipient (or one or more strength-enhancing excipients, or the strength-enhancing excipient in its totality) having a solubility in a relevant physiological/body fluid under physiological conditions no greater than 1 g/l, or no greater than 0.5 g/l, or no greater than 0.2 g/l, or no greater than 0.1 g/l, or no greater than 0.05 g/l, or no greater than 0.02 g/l, or no greater than 0.01 g/l, or no greater than 0.005 g/l, or no greater than 0.002 g/l, or no greater than 0.001 g/l. In the extreme case, strength-enhancing excipient (e.g., at least one strength-enhancing excipient or the strength-enhancing excipient in its totality) may be insoluble or at least practically insoluble in a relevant physiological fluid under physiological conditions.

It may be noted that even if the solubility of a relevant physiological fluid is low in a strength-enhancing excipient, said strength-enhancing excipient may soften or plasticize somewhat upon contact with or immersion in said physiological fluid under physiological conditions. As a result, at least a strength-enhancing excipient can be a solid in the dry state, but upon immersion in or exposure to a relevant physiological fluid (e.g., gastric fluid, etc.) under physiological conditions, it may transition to a semi-solid or viscoelastic material.

Generally, the mechanical properties (such as stiffness, yield strength, tensile strength, etc.) of physiological fluid-soaked strength-enhancing excipient should be large enough to stabilize or mechanically support the dosage form or drug-containing solid or framework. However, the stiffness, yield strength, tensile strength, etc. of physiological fluid-soaked strength-enhancing excipient should not be too large, so that the expansion of the dosage form or drug-containing solid or framework after exposure to said physiological fluid is not excessively impaired or constrained. Thus, strength-enhancing excipients that comprise or form a semi-solid material upon exposure to a relevant physiological fluid are typically preferred herein.

In some embodiments, physiological fluid-soaked strength-enhancing excipient (e.g., a film that is immersed in a relevant physiological fluid (e.g., acidic water) for so long that the water concentration in the film is roughly at equilibrium) comprises an elastic modulus, or an elastic-plastic modulus, or a plastic modulus greater than 0.02 MPa. This includes, but is not limited to physiological fluid-soaked strength-enhancing excipient (e.g., a film that was immersed in a relevant physiological fluid (e.g., acidic water) for so long that the water concentration in the film is roughly at equilibrium) comprising an elastic modulus, or an elastic-plastic modulus, or a plastic modulus greater than 0.05 MPa, or greater than 0.1 MPa, or greater than 0.2 MPa, or greater than 0.3 MPa, or greater than 0.4 MPa, or greater than 0.5 MPa, or greater than 0.6 MPa, or greater than 0.7 MPa, or greater than 0.8 MPa, or greater than 0.9 MPa, or greater than 1 MPa. In some embodiments, moreover, physiological fluid-soaked strength-enhancing excipient (e.g., a film that was immersed in a relevant physiological fluid (e.g., acidic water) for so long that the water concentration in the film is roughly at equilibrium) comprises an elastic modulus, or an elastic-plastic modulus, or a plastic modulus no greater than about 1000 MPa (e.g., no greater than 500 MPa, or no greater than 200 MPa, or no greater than 100 MPa, or no greater than 50 MPa, or no greater than 20 MPa, or no greater than 10 MPa). Preferably, an elastic modulus of a physiological fluid-soaked strength-enhancing excipient should be greater than about 0.1 MPa and no greater than about 500 MPa.

In some embodiments, moreover, physiological fluid-soaked (e.g., acidic water-soaked) strength-enhancing excipient (e.g., a film that was immersed in a relevant physiological fluid (e.g., acidic water) for so long that the water concentration in the film is roughly at equilibrium) comprises a yield strength greater than 0.005 MPa. This includes, but is not limited to physiological fluid-soaked strength-enhancing excipient (e.g., a film that was immersed in a relevant physiological fluid (e.g., acidic water) for so long that the water concentration in the film is roughly at equilibrium) comprising a yield strength greater than 0.0075 MPa, or greater than 0.01 MPa, or greater than 0.02 MPa, or greater than 0.05 MPa, or greater than 0.1 MPa, or greater than 0.2 MPa. In some embodiments, moreover, physiological fluid-soaked strength-enhancing excipient (e.g., a film that was immersed in a physiological fluid (e.g., acidic water) for so long that the water concentration in the film is roughly at equilibrium) comprises a yield strength no greater than 500 MPa (e.g., no greater than 200 MPa, or no greater than 100 MPa, or no greater than 75 MPa, or no greater than 50 MPa, or no greater than 20 MPa, or no greater than 10 MPa, or no greater than 5 MPa).

In some embodiments, moreover, physiological fluid-soaked strength-enhancing excipient (e.g., a film that was immersed in a relevant physiological fluid (e.g., acidic water) for so long that the water concentration in the film is roughly at equilibrium) comprises a tensile strength greater than 0.02 MPa. This includes, but is not limited to physiological fluid-soaked strength-enhancing excipient (e.g., a film that was immersed in a relevant physiological fluid (e.g., acidic water) for so long that the water concentration in the film is roughly at equilibrium) comprising a tensile strength greater than 0.05 MPa, or greater than 0.08 MPa, or greater than 0.1 MPa, or greater than 0.2 MPa, or greater than 0.3 MPa, or greater than 0.4 MPa, or greater than 0.5 MPa, or greater than 0.6 MPa. In some embodiments, moreover, physiological fluid-soaked strength-enhancing excipient (e.g., a film that was immersed in a relevant physiological fluid (e.g., acidic water) for so long that the water concentration in the film is roughly at equilibrium) comprises a tensile strength no greater than 500 MPa (e.g., no greater than 200 MPa, or no greater than 100 MPa, or no greater than 75 MPa, or no greater than 50 MPa, or no greater than 20 MPa, or no greater than 10 MPa).

In some embodiments, moreover, physiological fluid-soaked strength-enhancing excipient (e.g., a film that was immersed in a relevant physiological fluid (e.g., acidic water) for so long that the water concentration in the film is roughly at equilibrium) comprises a strain at fracture greater than 0.2. This includes, but is not limited to physiological fluid-soaked strength-enhancing excipient (e.g., a film that was immersed in a relevant physiological fluid (e.g., acidic water) for so long that the water concentration in the film was roughly at equilibrium) comprising a strain at fracture greater than 0.5, or greater than 0.75, or greater than 1, or greater than 1.25, or greater than 1.5, or greater than 1.75, or greater than 2, or greater than 2.25, or greater than 2.5. Preferably, the strain at fracture of a physiological fluid-soaked strength-enhancing excipient should be greater than about 1.

Furthermore, in some embodiments, the solubility of at least one strength-enhancing excipient (or the solubility of the strength-enhancing excipient in its totality) can differ in different physiological fluids under physiological conditions. By way of example but not by way of limitation, in some embodiments the solubility of at least one strength-enhancing excipient in aqueous physiological fluid may depend on the pH value of said physiological fluid. More specifically, in some embodiments at least one strength-enhancing excipient can be sparingly-soluble or insoluble or practically insoluble in an aqueous physiological fluid that is acidic (e.g., in gastric fluid, or in fluid with a pH value smaller than about 4, or in fluid with a pH value smaller than about 5, etc.), but it can be soluble in an aqueous physiological fluid having a greater pH value (e.g., in a fluid with a pH value greater than about 6, or greater than about 6.5, or greater than about 7, or greater than about 7.5, etc.), such as intestinal fluid. A strength-enhancing excipient comprising a solubility that is smaller in acidic solutions than in basic solutions is also referred to herein as “enteric excipient”.

In some embodiments, therefore, at least one strength-enhancing excipient comprises a solubility in aqueous fluid with a pH value no greater than 4 at least 10 (e.g., at least 20, or at least 50, or at least 100, or at least 200, or at least 500) times smaller than the solubility of said strength-enhancing excipient in an aqueous fluid with pH value greater than 7.

A non-limiting example of such a strength-enhancing excipient that is sparingly-soluble in gastric or acidic fluid, but dissolves in intestinal fluid (e.g., aqueous fluid with a pH value greater than about 5.5), is methacrylic acid-ethyl acrylate copolymer.

Other non-limiting examples of strength-enhancing excipients herein may include hydroxypropyl methyl cellulose acetate succinate, methacrylic acid-ethyl acrylate copolymer, methacrylate-copolymers (e.g., poly(ethyl acrylate-co-methyl methacrylate-co-trimethylammonioethyl methacrylate chloride) 1:2:0.2, poly(ethyl acrylate-co-methyl methacrylate-co-trimethylammonioethyl methacrylate chloride) 1:2:0.1, Poly(ethyl acrylate-co-methyl methacrylate) 2:1, etc.), and so on.

### (h) Expansion of Drug-Containing Solid and Formation of a Semi-Solid Mass

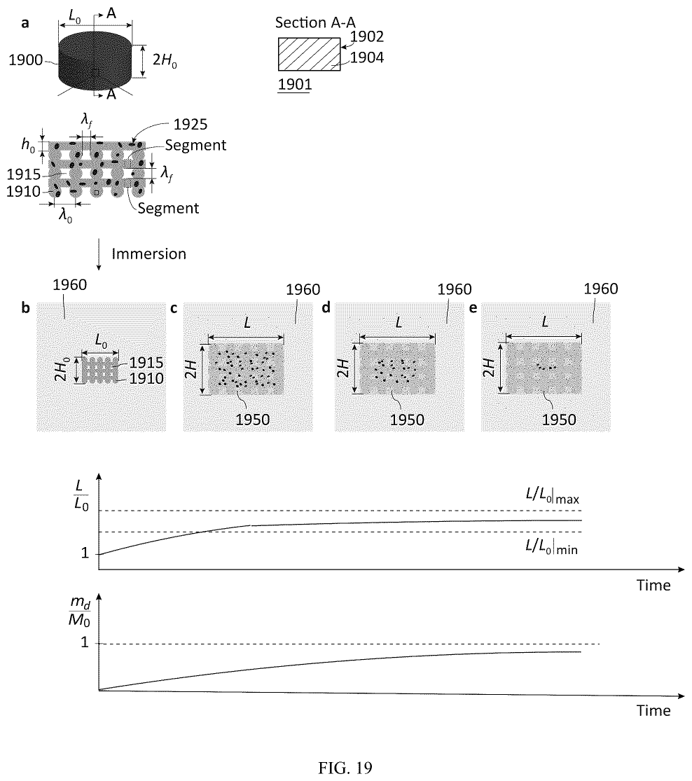

FIG. 19 presents a non-limiting example of a pharmaceutical dosage form 1900 comprising a drug-containing solid 1901 having an outer surface 1902 and an internal three dimensional structural framework 1904 of one or more substantially orderly arranged thin structural elements 1910. The framework 1904 is contiguous with and terminates at said outer surface 1902. The structural elements comprise a hydrophilic surface composition. The structural elements 1910 further comprise segments spaced apart from adjoining segments 1910, thereby defining free spaces 1915. A plurality of adjacent free spaces 1915 may combine to define one or more interconnected, free spaces 1915 forming an open pore network that extends over a length at least half the thickness of the drug-containing solid 1901 (e.g., over the entire length, width, and thickness of the drug-containing solid and terminating at its outer surface 1902) or over a length at least twice the thickness of one or more elements 1910. The structural elements 1910 further comprise at least one active ingredient (e.g., at least one drug) dissolved as drug molecules 1920 or dispersed as particles in an excipient matrix 1930, 1950. Thus the drug forms a solid solution or a solid dispersion with said excipient matrix 1930, 1950. The excipient matrix 1930, 1950 comprises at least an absorptive polymeric excipient 1930 and at least a strength-enhancing polymeric excipient.

Upon immersion in a relevant physiological fluid, said fluid percolates interconnected free space and diffuses into one or more said elements, so that the framework expands in all dimensions. Because dosage forms (or drug-containing solids) herein may comprise a structural framework of thin elements with hydrophilic surface composition surrounded by interconnected free space that may terminate at the outer surface of the drug-containing solid, the rates of fluid percolation and diffusion, and consequently also the rate of expansion of the drug-containing solid or three dimensional structural framework of elements can be substantial.

In some embodiments of the invention herein, accordingly, at least one dimension (e.g., a side length or the thickness) of the drug-containing solid expands to at least 1.3 times the initial value (e.g., the initial length prior to exposure to said physiological fluid) as it transitions to a fluidic or viscous medium within no more than 300 minutes of immersion in a physiological or body fluid under physiological conditions. This includes, but is not limited to at least one dimension of the drug-containing solid reaching a length at least 1.3 times the initial length within no more than 250 minutes, or within no more than 200 minutes, or within no more than 150 minutes, or within no more than 100 minutes, or within no more than 50 minutes, or within no more than 40 minutes, or within no more than 30 minutes, or within no more than 20 minutes of immersion in said physiological or body fluid under physiological conditions. This may also include, but is not limited to at least one dimension of the drug-containing solid or framework expanding to a length at least 1.35 times the initial length, or at least 1.4 times the initial length, or at least 1.45 times the initial length, or at least 1.5 times the initial length, or at least 1.55 times the initial length, or at least 1.6 times the initial length, or at least 1.65 times the initial length within no more than 300 minutes of immersing in or exposing to a physiological or body fluid under physiological conditions.

Furthermore, in some embodiments the drug-containing solid expands to at least 2 times its initial volume within no more than about 300 minutes of immersing in a physiological or body fluid under physiological conditions. This includes, but is not limited to a drug-containing solid that expands to at least 3 times, or at least 4 times, or at least 4.5 times, or at least 5 times, or at least 5.5 times, or at least 6 times, or at least 6.5 times its initial volume within no more than about 300 minutes of immersing in a physiological or body fluid under physiological conditions.

In some embodiments, the drug-containing solid (or the three dimensional structural framework) expands isotropically (e.g., uniformly in all directions) while transitioning to a semi-solid mass. In the invention herein, a solid mass is generally understood to expand isotropically if the normalized expansion (e.g., the ratio of a length difference and the initial length, such as (L(t)−L₀)/L₀, (H(t)−H₀)/H₀, etc.) deviates by less than about 50-75 percent of its maximum value by changing direction or orientation. Thus, in an isotropically expanding solid, semi-solid mass, or framework, the normalized expansion is roughly the same in all directions. FIG. 19 shows a non-limiting schematic illustration of a drug-containing solid that expands isotropically. For further information related to isotropic expansion of a drug-containing solid, see, e.g., the International Application No. PCT/US19/19004 filed on Feb. 21, 2019 and titled “Expanding structured dosage form”.

In some embodiments, upon prolonged exposure to a physiological fluid (e.g., longer than 2, 4, 6, 8, or 10 hours in a lightly stirred dissolution fluid such as acidic water), said expanded framework or semi-solid mass maintains its length between 1.3 and 4 times the initial length for prolonged time.

In some embodiments, the semi-solid mass comprises a substantially continuous or connected network of one or more strength-enhancing excipients.

In some embodiments, the semi-solid mass comprises a substantially continuous or connected network of strength-enhancing excipient that extends over the length, width, and thickness of said semi-solid mass.

### j) Mechanical Properties of Expanded Semi-Solid Mass

In some embodiments, moreover a semi-solid mass (e.g., an expanded drug-containing solid or dosage form) formed after immersion of a drug-containing solid in a physiological fluid under physiological conditions comprises an elastic modulus greater than 0.005 MPa. This includes, but is not limited to a viscous or semi-solid mass (e.g., an expanded drug-containing solid or dosage form) formed after immersion of a drug-containing solid in a dissolution fluid comprising an elastic modulus greater than 0.007 MPa, or greater than 0.01 MPa, or greater than 0.015 MPa, or greater than 0.02 MPa, or greater than 0.025 MPa, or greater than 0.03 MPa, or greater than 0.035 MPa, or greater than 0.04 MPa, or greater than 0.045 MPa, or greater than 0.05 MPa, or greater than 0.055 MPa, or greater than 0.06 MPa, or greater than 0.065 MPa, or greater than 0.07 MPa, or greater than 0.075 MPa. In some embodiments, therefore, a viscous or semi-solid mass (e.g., an expanded drug-containing solid or dosage form) formed after immersion of a drug-containing solid in a dissolution fluid is a highly elastic mass or semi-solid or structure that may not break or permanently deform for prolonged time in a stomach (e.g., under the compressive forces of stomach walls, etc.).

In some embodiments, moreover a viscous or semi-solid mass (e.g., an expanded drug-containing solid or dosage form) formed after immersion of a drug-containing solid in a dissolution fluid comprises an elastic modulus no greater than 50 MPa (e.g., no greater than 40 MPa, or no greater than 30 MPa, or no greater than 20 MPa, or no greater than 10 MPa, or no greater than 5 MPa).

In some embodiments, moreover a semi-solid mass formed after immersion of a drug-containing solid in a dissolution fluid comprises a yield strength or a fracture strength greater than 0.002 MPa. This includes, but is not limited to a viscous or semi-solid mass formed after immersion of a drug-containing solid in a dissolution fluid comprising a yield strength or a fracture strength greater than 0.005 MPa, or greater than 0.007 MPa, or greater than 0.01 MPa, or greater than 0.02 MPa, or greater than 0.025 MPa, or greater than 0.03 MPa, or greater than 0.035 MPa, or greater than 0.04 MPa, or greater than 0.045 MPa, or greater than 0.05 MPa, or greater than 0.055 MPa, or greater than 0.06 MPa, or greater than 0.065 MPa, or greater than 0.07 MPa, or greater than 0.075 MPa, or greater than 0.8 MPa.

In some embodiments, moreover a semi-solid mass (e.g., an expanded drug-containing solid or dosage form) formed after immersion of a drug-containing solid in a dissolution fluid comprises a yield strength or a fracture strength no greater than 50 MPa (e.g., no greater than 20 MPa, or no greater than 10 MPa, or no greater than 5 MPa, or no greater than 2 MPa, or no greater than 1 MPa).

In some embodiments, therefore, upon ingestion the dosage form is retained in the stomach for a prolonged time to deliver drug into the blood stream over a prolonged time (e.g., 80 percent of the drug is released in 30 mins-200 hours, 1 hour to 200 hours; 1 hour-150 hours; 3 hours-200 hours; 5 hours-200 hours; 3 hours-60 hours; 5 hours-60 hours; 2 hours-30 hours; 5 hours-24 hours; 30 mins-96 hours, 30 mins-72 hours, 30 mins-48 hours, 30 mins-36 hours, 30 mins-24 hours, 1-10 hours, 45 min-10 hours, 30 min-10 hours, 45 min-8 hours, 45 min-6 hours, 30 min-8 hours, 30 min-6 hours, 30 min-5 hours, 30 min-4 hours, etc.) and at a precisely controlled rate. This enables improved control of drug concentration in the blood stream, and improved efficacy or reduced side effects of numerous drug therapies.

### j) Drug Release Properties of Drug-Containing Solid, Dosage Form, and Viscous Mass

In some embodiments, moreover, eighty percent of the drug content in the drug-containing solid is released in more than 30 minutes after immersion in a physiological or body fluid under physiological conditions. This includes, but is not limited to a drug-containing solid that releases eighty percent of the drug content in more than than 40 minutes, or in more than 50 minutes, or in more than 60 minutes, or in more than 100 minutes, or in 30 minutes-150 hours, 30 minutes-48 hours, 30 minutes-36 hours, or 45 minutes-24 hours after immersion in a physiological fluid under physiological conditions.

### k) Mechanical Properties of Drug-Containing Solid and Solid Dosage Form

In some embodiments the tensile strength of a drug-containing solid or a three dimensional structural framework of one or more elements is between 0.01 MPa and 100 MPa (this includes, but is not limited to tensile strength of at least one element is greater than 0.02 MPa, or greater than 0.05 MPa, or greater than 0.1 MPa, or greater than 0.2 MPa, or greater than 0.5 MPa, or greater than 1 MPa, or greater than 1.5 MPa, or greater than 2 MPa, or greater than 3 MPa, or greater than 5 MPa).

Finally, in some embodiments the tensile strength of a drug-containing solid or a three dimensional structural framework of one or more elements is between 0.01 MPa and 100 MPa (this includes, but is not limited to tensile strength of at least one element is greater than 0.02 MPa, or greater than 0.05 MPa, or greater than 0.1 MPa, or greater than 0.2 MPa, or greater than 0.5 MPa, or greater than 1 MPa, or greater than 1.5 MPa, or greater than 2 MPa, or greater than 3 MPa, or greater than 5 MPa).

### EXPERIMENTAL EXAMPLES

### Part 1

The following examples present ways by which the fibrous dosage forms may be prepared and analyzed, and will enable one of skill in the art to more readily understand the principle thereof. The examples are presented by way of illustration and are not meant to be limiting in any way.

### Example 1.1: Preparation of Fibrous Dosage Forms

The non-limiting experimental examples 1-7 refer to single fibers and fibrous dosage forms consisting of 20 wt % ibuprofen drug, 60 wt % hydroxypropyl methyl cellulose (HPMC) with a molecular weight of 120 kg/mol (an absorptive excipient), and 20 wt % methacrylic acid-ethyl acrylate copolymer (1:1) with a molecular weight of about 250 kg/mol (a strength-enhancing and enteric excipient, also referred to herein as “Eudragit L100-55”).

To prepare the dosage forms, ibuprofen drug particles were first dissolved in dimethyl sulfoxide (DMSO) solvent to form a uniform solution with a drug concentration of 60 mg/ml DMSO. Then the ibuprofen-DMSO solution was mixed with the excipients (75 wt % hydroxypropyl methylcellulose (HPMC) with a molecular weight of 120 kg/mol and 25 wt % Eudragit L100-55) at the ratio 240 mg excipient/ml DMSO.

The mixture was extruded through a laboratory extruder to form a uniform viscous paste. The viscous paste was then put in a syringe equipped with a hypodermic needle of inner radius, Rₙ=76 μm. The paste was extruded through the needle to form a wet fiber that was either deposited as a single fiber or as a fibrous dosage form with cross-ply structure as in previous disclosures (for further details, see, e.g., the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, or the International Application No. PCT/US19/52030 filed on Sep. 19, 2019 and titled “Dosage form comprising structured solid-solution framework of sparingly-soluble drug and method for manufacture thereof”).

As mentioned above and listed in Table 1, single fibers with nominal fiber radius, Rₙ=76 μm, were deposited. Also, three dosage forms with the same Rₙ and the nominal inter-fiber spacing,λₙ=1250 μm (dosage form A), 500 μm (dosage form B), and 350 μm (dosage form C) were prepared.

After depositing or patterning, to solidify the fibers and the dosage forms, the solvent was evaporated by blowing warm air at about 40-60° C. and 1 m/s over them for a day. Post evaporation, the solvent concentration in the solid fibers and dosage forms was below the limit specified by the regulatory authorities, 0.5 wt %.

Finally, the solid dosage forms were trimmed with a microtome blade to square disks of nominal dimensions about 7.5 mm×7.5 mm×2 mm.

### Example 1.2: Estimation of Microstructural Parameters

A non-limiting example to estimate some microstructural parameters of the dosage forms is as follows. Under the rough assumption that the fibers and dosage forms contract isotropically during solvent evaporation, the radius, R₀, and length or inter-fiber spacing, λ₀, of the solid fibers and the solid dosage forms may be derived as:

$$\frac{{R}_{0}}{{R}_{n}} = \frac{{λ}_{0}}{{λ}_{n}} = {( 1 - \frac{{c}_{solv}}{{ρ}_{solv}} )}^{1 / 3} \qquad ( 32 )$$

where Rₙ is the nominal fiber radius, λₙ the nominal inter-fiber spacing, cₛₒₗᵥ the concentration of solvent in the wet fiber (e.g., in the viscous paste during depositing or micro-patterning the fibers), and ρₛₒₗᵥ is the density of the solvent (e.g., DMSO).

The volume fraction of fibers in the solid cross-ply structure of the dosage forms may be expressed as (for further details, see, e.g., A. H. Blaesi, N. Saka, Mater. Sci. Eng. C (2021) 110211, and references therein):

$$φ = ξ \frac{π {R}_{0}}{2 {λ}_{0}} \qquad ( 33 a )$$

where ξ is the ratio of the “nominal” thickness of the dosage form (point contacts between fibers) and the “real” thickness of the dosage form (flattened fiber-to-fiber contacts):

$$ξ = \frac{2 {R}_{0} {n}_{l}}{2 {H}_{0}} = \frac{{R}_{0} {n}_{l}}{{H}_{0}} \qquad ( 33 b )$$

Here nₗ is the number of stacked layers, and H₀ the half-thickness of the solid dosage form with flattened contacts.

Table 1 below lists the nominal and estimated microstructural parameters of the various dosage forms prepared as described in the non-limiting experimental example 1.1.

TABLE 1
Microstructural parameters of single fibers
and fibrous dosage forms.

| | Rₙ | λₙ | | R₀ | λ₀ | |
|---|---|---|---|---|---|---|
| | (μm) | (μm) | 2Rₙ/λₙ | (μm) | (μm) | φ |
| Single fibers | 76 | — | — | 46 | — | — |
| Fibrous dosage forms | | | | | | |
| A | 76 | 1250 | 0.12 | 46 | 763 | 0.16 |
| B | 76 | 500 | 0.30 | 46 | 305 | 0.39 |
| C | 76 | 350 | 0.43 | 46 | 214 | 0.56 |

Rₙ: nominal fiber radius; λₙ: nominal inter-fiber distance; R₀: solid fiber radius; λ₀: inter-fiber distance in solid dosage form; φ: fiber volume fraction in solid dosage form.
R₀ and λ₀ were calculated by Eq. (32) using cₛₒₗᵥ = 850 mg/ml and pₛₒₗᵥ = 1100 mg/ml.
φ was calculated by Eq. (33) using ξ = R₀n_{layers}/H₀ ≈ 1.65, where R₀ = 46 μm, n₁ = 36, and H₀ ≈ 1 mm.

### Example 1.3: Measurement of Microstructural Parameters

A fiber and a dosage form were imaged by a Zeiss Merlin High Resolution SEM with a GEMINI column. Images were taken without any preparation of the sample. Imaging was done with an in-lens secondary electron detector. An accelerating voltage of 5 kV and a probe current of 95 pA were applied to operate the microscope.

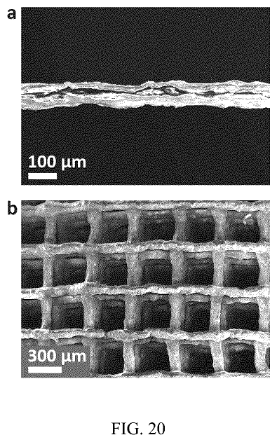

FIG. 20a is a scanning electron micrograph of the single fiber. The average fiber radius was about 49.5 μm. FIG. 20b presents a top view of the microstructure of a fibrous dosage form B. The fiber radius, R₀=45.2±6 μm and the inter-fiber distance, λ₀=390.3±18 μm. Both values are in rough agreement with the estimated values presented in Table 1.

It may be noted, furthermore, that cross-sectional images of the fibers or dosage forms may be taken to further characterize the microstructures. (For non-limiting examples of cross sectional images of fibrous cross-ply structures, see, e.g., the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, or the International Application No. PCT/US19/52030 filed on Sep. 19, 2019 and titled “Dosage form comprising structured solid-solution framework of sparingly-soluble drug and method for manufacture thereof”).

### Example 1.4: Expansion of Single Fibers

To determine the expansion rate of a single fiber, the fiber was immersed in a beaker filled with 400 ml dissolution fluid (0.1 M hydrogen chloride (HCl) in deionized water at a temperature of 37° C.). The fluid was stirred with a paddle rotating at 50 rpm. The immersed sample was continuously imaged by a Nikon DX camera.

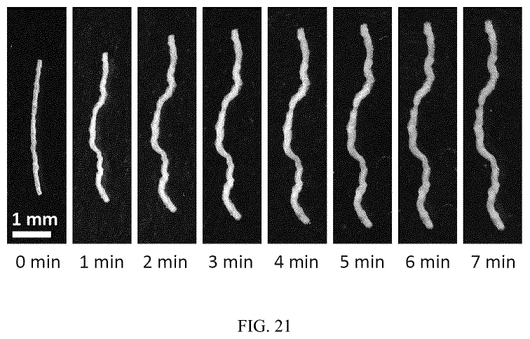

Images of the single fiber at various times after immersion in the dissolution fluid are shown in FIG. 21. The fiber transitioned from solid to semi-solid or viscous and expanded in both radial and axial directions. The integrity of the expanded fiber was preserved for more than an hour.

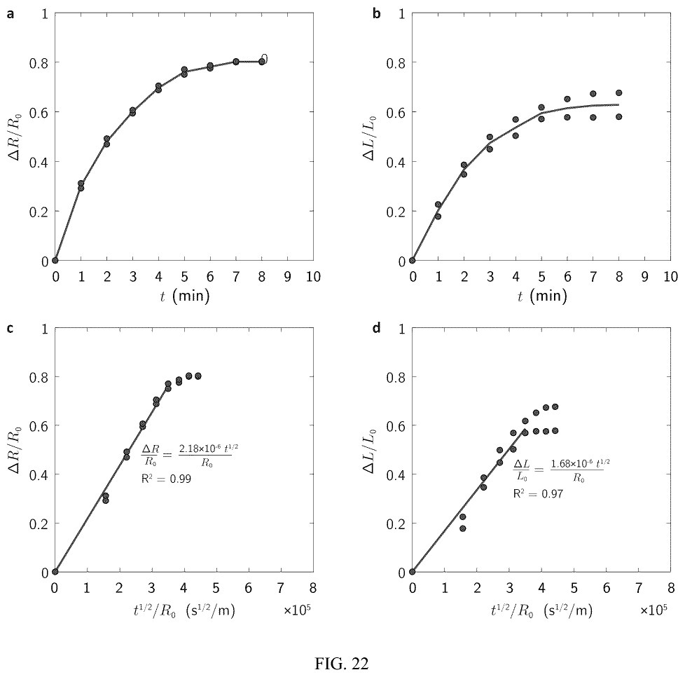

FIGS. 22a and 22b present plots of the normalized radial and axial expansions, ΔR/R₀ and ΔL/L₀, of the single fiber versus time. Both ΔR/R₀ and ΔL/L₀ steadily increased with time, but at a decreasing rate. The radial expansion was slightly greater than the axial expansion. Eight minutes after immersion, ΔR/R₀ was 0.8 and ΔL/L₀ was about 0.63.

FIGS. 22c and 22d are plots of the normalized radial and axial expansions of the single fiber versus t^{1/2}/R₀. In agreement with the model Eq. (7), for short times (e.g., t≤5 min):

$$\frac{Δ R}{{R}_{0}} = {{k}_{R} ( \frac{t}{{R}_{0}^{2}} )}^{1 / 2} and \qquad ( 34 a )$$

$$\frac{Δ L}{{L}_{0}} = {{k}_{L} ( \frac{t}{{L}_{0}^{2}} )}^{1 / 2} \qquad ( 34 b )$$

where k_{R} and k_{L}, respectively, are radial and longitudinal expansion rate constants.

From Eqs. (34) and (7) the diffusivity of dissolution fluid in the fiber may be estimated as:

$${D}_{w} = \frac{9 π}{16} {( \frac{{ρ}_{w}}{{c}_{b}} )}^{2} {k}_{R}^{2} and \qquad ( 35 a )$$

$${D}_{w} = \frac{9 π}{16} {( \frac{{ρ}_{w}}{{c}_{b}} )}^{2} {k}_{L}^{2} \qquad ( 35 b )$$

Using ρ_{w}/c_{b}˜1 and the k_{R} and k_{L} values from FIG. 22, by Eq. (35a) D_{w}˜1.76×10⁻¹¹ m²/s, and by Eq. (35b) D_{w}˜1.05×10⁻¹¹ m²/s.

### Example 1.5: Expansion of Fibrous Dosage Forms

To determine the expansion rate of fibrous dosage forms, the dosage form was immersed in a beaker filled with 400 ml dissolution fluid (0.1 M HCl in deionized water at 37° C.). The fluid was stirred with a paddle rotating at 50 rpm. The immersed sample was continuously imaged by a Nikon DX camera.

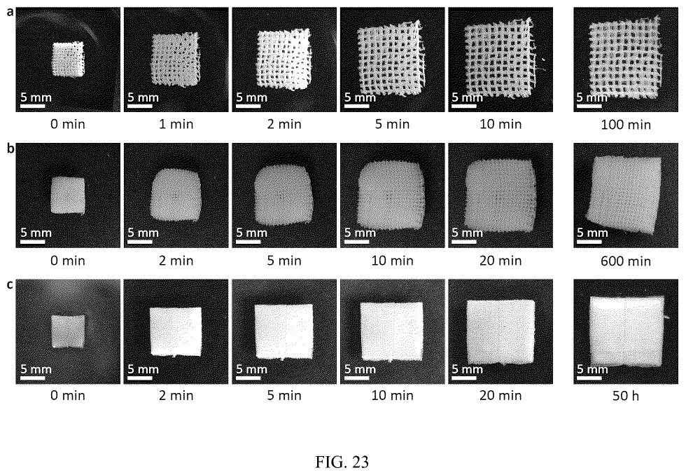

For all dosage forms A, B, and C, upon immersion of the dosage form in the dissolution fluid, the fluid percolated the inter-fiber void space rapidly. The solid dosage form then expanded isotropically and transformed into a highly viscous or semi-solid mass, FIG. 23. The geometry of the expanded viscous or semi-solid masses (or the expanded viscous or semi-solid dosage forms) A, B, and C was stabilized by the enteric excipient and preserved or maintained for more than 2, 10, and 50 hours, respectively.

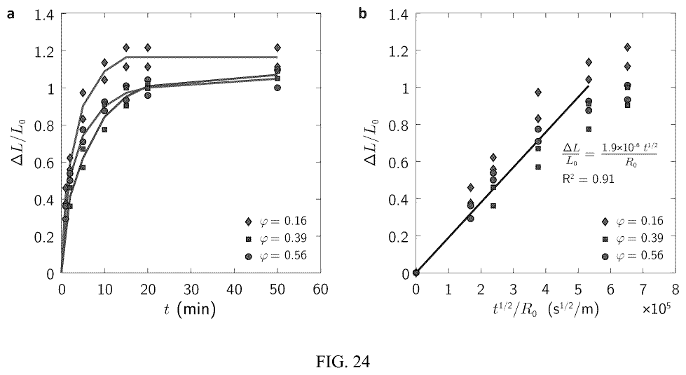

FIG. 24a is a plot of the normalized longitudinal expansion, ΔL/L₀, versus time. The ratio ΔL/L₀ increased with time at decreasing rate. The ratio of the dosage form length after 15 minutes and the initial length, L₁₅/L₀, was about two, Table 2. After about 20 minutes, the dosage form did not expand any further.

FIG. 24b is a plot of ΔL/L₀ versus t^{1/2}/R₀. In agreement with Eqs. (7) and (8), ΔL/L₀ was proportional to t^{1/2}/R₀ initially. Thus

$$\frac{Δ L}{{L}_{0}} ≅ {{k}_{e x} ( \frac{t}{{R}_{0}^{2}} )}^{1 / 2} \qquad ( 36 )$$

where kₑₓ is an expansion rate constant.

From FIGS. 23 and 24, kₑₓ was about the same as k_{L} and k_{R} of the single fiber. The constants, k_{LL}=kₑₓ/k_{L}˜1.2, and k_{RL}=kₑₓ/k_{R}˜0.92.

Thus, the normalized longitudinal expansion rate of the dosage forms was about the same as the normalized axial and radial expansion rates of the single fibers.

### Example 1.6: Drug Release by Single Fibers

Drug release by single fibers was monitored using the same setup and under the same conditions as in Example 1.4. In addition, at regular time intervals an aliquot of the dissolution fluid was sampled, and its UV absorbance spectrum was measured using a Perkin Elmer Lambda 950 UV/Vis Spectrophotometer. The fraction of drug released was determined by subtracting the UV absorbance at a wavelength of 235 nm from that at 230 nm, and dividing the resulting value with the value obtained at “infinite” time (i.e., when all drug was dissolved).

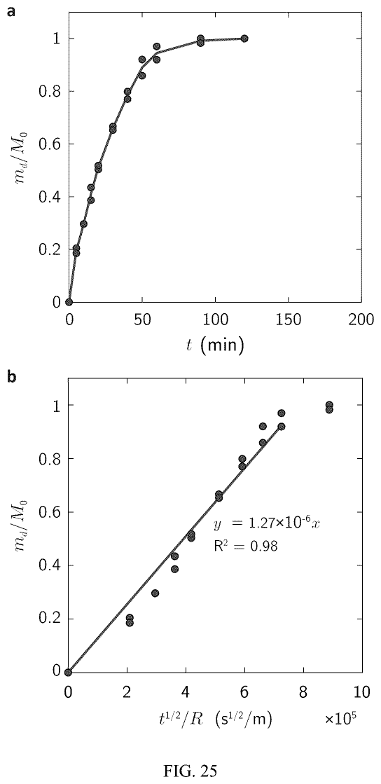

The fraction of drug released by single fibers, m_{d}/M₀, is plotted versus time, t, in FIG. 25a. The time to release 80 percent of the initial amount of drug, t_{0.8}, was 42 minutes. Moreover, as predicted in the modeling section, the fraction of drug released obeyed an equation of the form (FIG. 25b):

$$\frac{{m}_{d}}{{M}_{0}} = {{k}_{d} ( \frac{t}{{R}^{2}} )}^{1 / 2} \qquad ( 37 )$$

where k_{d} is a drug release rate constant.

From Eqs. (24) and (37) the diffusivity of drug through the expanded fiber may be written as:

$${D}_{d} = \frac{( {c}_{d , 0} - {c}_{s} ) {k}_{d}^{2}}{4 {c}_{s}} \qquad ( 38 )$$

For the non-limiting parameters c_{d,0}˜37.9 mg/ml, cₛ˜0.05 mg/ml, and k_{d}˜1.27×10⁻⁶ m/s^{1/2} (FIG. 25), the diffusivity, D_{d}˜3×10⁻¹⁰ m²/s. This is about half of that of the drug molecules in water. Thus, neither the absorptive excipient nor the strength-enhancing excipient appeared to substantially block drug diffusion out.

TABLE 2
Microstructural parameters and expansion and drug release properties
of single fibers and fibrous dosage forms.

| | R₀ | λ₀ | | | | t_{0.8} |
|---|---|---|---|---|---|---|
| | (μm) | (μm) | 2R₀/λ₀ | φ | L₁₅/L₀ | (min) |
| Single fibers | 46 | — | — | — | — | 42 |
| Fibrous dosage forms | | | | | | |
| A | 46 | 763 | 0.12 | 0.16 | 2.2 | 120 |
| B | 46 | 305 | 0.3 | 0.39 | 1.95 | 620 |
| C | 46 | 214 | 0.43 | 0.56 | 2.0 | 2280 |

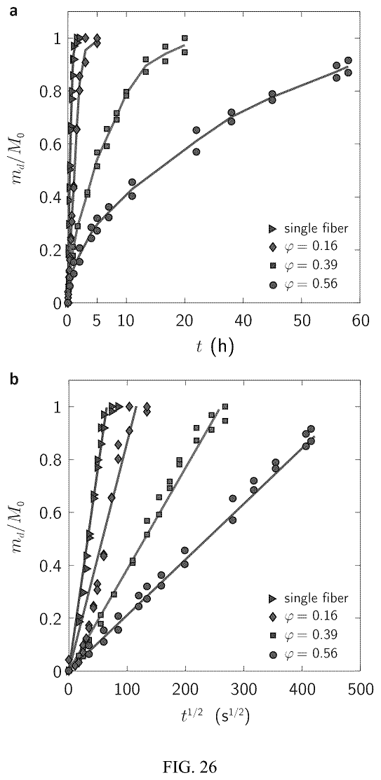

R₀: solid fiber radius; λ₀: inter-fiber distance in solid dosage form; φ: fiber volume fraction in solid dosage form; L₁₅/L₀: ratio of the side length at 15 minutes and the initial length; t_{0.8}: time to release 80% of the drug content.
R₀, λ₀, and φ are as derived in Table 1.
L₁₅/L₀ is the average of two samples obtained from the results shown in FIG. 24a. The values of the individual samples were 2.216 and 2.113 (A), 2 and 1.903 (B), and 2.011 and 1.933 (C).
t_{0.8} is the average of two samples obtained from the results shown in FIG. 26a. The values of the individual samples were 42 and 43 min (single fiber), 112 and 120 min (A), 600 and 640 min (B), and 37 and 38 h (C).

### Example 1.7: Drug Release by Fibrous Dosage Forms

Drug release by fibrous dosage forms was monitored using the same setup and under the same conditions as in Example 1.5. In addition, at regular time intervals an aliquot of the dissolution fluid was sampled, and its UV absorbance spectrum was measured using a Perkin Elmer Lambda 950 UV/Vis Spectrophotometer. The fraction of drug released was determined by subtracting the UV absorbance at a wavelength of 235 nm from that at 230 nm, and dividing the resulting value with the value obtained at “infinite” time (i.e., when all drug was dissolved).

FIG. 26a is a plot of the fraction of drug released by the dosage forms versus time. The t_{0.8} times of the dosage forms with fiber volume fractions, φ=0.16, 0.39, and 0.56 were 2, 10, and 38 hours, respectively, Table 2. Thus, t_{0.8} increased greatly with φ.

The derivation of analytical equations for calculating the fraction of drug released and the t_{0.8} time of fibrous dosage forms is beyond the scope of this disclosure. However, semi-analytical equations may be obtained as shown below.

From the drug release models shown in the section “Models of expansion, drug release, and disintegration of the dosage form”, if the initial drug concentration in the expanded fibers, c_{d,0}>>cₛ, the solubility, the fraction of drug released by the single fiber (φ=0) may be written as:

$$\frac{{m}_{d} ( φ = 0 )}{{M}_{0}} = 2 {( \frac{{c}_{s} {D}_{d} t}{{c}_{d , 0} {R}^{2}} )}^{1 / 2} \qquad ( 39 a )$$

and that by the monolithic dosage form (φ=1) may be expressed as:

$$\frac{{m}_{d} ( φ = 1 )}{{M}_{0}} = \sqrt{2} {( \frac{{c}_{s} {D}_{d} t}{{c}_{d , 0} {H}^{2}} )}^{1 / 2} \qquad ( 39 b )$$

Both Eqs. (39a) and (39b) are of the same form. Thus, the fraction of drug released by the fibrous dosage form may follow the equation:

$$\frac{{m}_{d} ( φ )}{{M}_{0}} = κ ( φ ) {( \frac{{c}_{s} {D}_{d} t}{{c}_{d , 0} {ζ ( φ )}^{2}} )}^{1 / 2} \qquad ( 40 )$$

where κ(φ) is a dimensionless constant and ζ(φ) a diffusion length.

The constant κ(φ) may be assumed to follow the weighted geometric mean of the constant of the single fiber (κ=2) and that of the monolithic slab (κ=√2):

κ(φ)=2^{1-φ}\sqrt{2}^{φ} (41a)

Similarly, ζ(φ) may be assumed to be the weighted geometric mean of the fiber radius, R, and the half-thickness of the monolithic dosage form, H:

ζ(φ)=R^{1-φ}H^{φ} (41b)

Substituting Eqs. (41a) and (41b) in Eq. (40) gives:

$$\frac{{m}_{d} ( φ )}{{M}_{0}} = ( \frac{2}{{\sqrt{2}}^{φ}} ) {( \frac{{c}_{s}}{{c}_{d , 0}} )}^{1 / 2} \frac{{D}_{d}^{1 / 2} {t}^{1 / 2}}{{R}^{1 - φ} {H}^{φ}} \qquad ( 42 )$$

FIG. 26b plots the calculated curves of m_{d}/M₀ versus t^{1/2} for the non-limiting experimental parameters (cₛ=0.05 mg/ml, c_{d,0}=37.9 mg/ml, D_{d}=3.24×10⁻¹⁰ m²/s, R=83 μm, H=2 mm), and compares them with the experimental data points. Indeed, for all dosage forms, the calculated and measured values agree. Thus, the model may be valid.

The time to release eighty percent of the drug content may be obtained by substituting m_{d}/M₀=0.8 in Eq. (42) and rearranging as:

$${t}_{0.8} ( φ ) = 0.16 \times {2}^{φ} ( \frac{{c}_{d , 0}}{{c}_{s}} ) {( \frac{{R}^{1 - φ} {H}^{φ}}{{D}_{d}^{1 / 2}} )}^{2} \qquad ( 43 )$$

Thus, t_{0.8} may scale with the square of the weighted diffusion length, R^{1-φ}H^{φ}.

By simplifying Eq. (16), the t_{0.8} time may be written as an exponential function of φ as:

$${t}_{0.8} ( φ ) = 0 \text{.16} ( \frac{{c}_{d , 0}}{{c}_{s}} ) ( \frac{{R}^{2}}{{D}_{d}} ) {( \frac{\sqrt{2} H}{R} )}^{2 φ} \qquad ( 44 )$$

Taking the logarithm on both sides of Eq. (44) and rearranging,

$$ln ( {t}_{0.8} ( φ ) ) = ln ( 0 \text{.16} ( \frac{{c}_{d , 0}}{{c}_{s}} ) ( \frac{{R}^{2}}{{D}_{d}} ) ) + φln ( \frac{2 {H}^{2}}{{R}^{2}} ) \qquad ( 45 )$$

Exponentiating and rearranging again, Eq. (45) may be rewritten as:

$${t}_{0.8} ( φ ) = α exp ( β φ ) where \qquad ( 46 a )$$

$$α = 0. 16 ( \frac{{c}_{d , 0}}{{c}_{s}} ) ( \frac{{R}^{2}}{{D}_{d}} ) \qquad ( 46 b )$$

$$β = ln ( \frac{2 {H}^{2}}{{R}^{2}} ) \qquad ( 46 c )$$

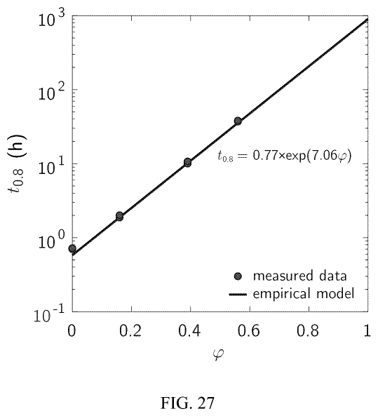

FIG. 27 plots the calculated values of log(t_{0.8}) versus φ for the relevant experimental parameters (c_{d,0}=37.9 mg/ml, cₛ=0.05 mg/ml, R=83 μm, D_{d}=3.24×10⁻¹⁰ m²/s H=2 mm), and compares them with the experimental data points. The calculated and measured values roughly agree.

Thus, by varying φ the t_{0.8} time of the non-limiting experimental fibrous dosage forms increased exponentially from that of the thin, single fibers to that of the thick, monolithic dosage form.

### Example 1.8: Diffusivity of Absorptive Excipient (e.g., HPMC 120 k) Through the Disintegrating Single Fiber

A single fiber of radius ˜80 μm was immersed in a dissolution fluid (deionized water with 0.1 M HCl at 37 degree Celsius) that was stirred with a paddle rotating at 50 rpm. The fiber was removed from the dissolution bath at specific time points, and the weight of the disintegrating fiber was determined by a Mettler Toledo analytical balance.

In the experiments, the time to remove 63 percent of the initial weight of HPMC excipient in the fiber was greater than 8 hours.

For an approximate, order-of-magnitude analysis of the diffusivity of absorptive excipient through the expanded fiber, the diffusivity of absorptive excipient molecules, Dₐₑ, through the fiber is assumed constant. The absorptive excipient concentration in the expanded fiber, cₐₑ(t), may then be governed by:

$$\frac{\partial {c}_{a e}}{\partial t} = {D}_{a e} \frac{{\partial }^{2} {c}_{a e}}{\partial {r}^{2}} r \leq {R}_{f} \qquad ( 47 a )$$

subject to the initial and boundary conditions:

cₐₑ=c₀ r≤R_{f} t=0 (47b)

cₐₑ=0 r=R_{f} (47c)

where r is the radial coordinate, t is time, R_{f} the radius of the expanded fiber, and c₀ is the initial concentration of absorptive excipient in the expanded fiber.

According to Crank, an analytical solution of Eq. (47) may be written as:

$$\frac{{c}_{0} - {c}_{a e}}{{c}_{0}} = 1 - 2 {\sum }_{i = 1}^{∞} \frac{{J}_{0} ( r {β}_{i} / {R}_{f} )}{{β}_{i} {J}_{1} ( {β}_{i} )} exp ( - {β}_{i}^{2} {D}_{a e} t / {R}_{f}^{2} ) \qquad ( 48 )$$

where the βᵢ's are the roots of

J₀(βᵢ)=0 (49)

Here J₀ is the Bessel function of the first kind of order zero.

The ratio of the mass of absorptive excipient in the fiber at time t, M(t), to the mass at t=0, M₀, may then be approximated by an adapted form of the equation presented by Crank:

$$\frac{M ( t )}{{M}_{0}} = {\sum }_{i = 1}^{∞} \frac{4}{{β}_{i}^{2}} exp ( - {β}_{i}^{2} {D}_{ae} t / {R}_{f}^{2} ) \qquad ( 50 a )$$

Substituting only the first root, β₁=2.4, gives:

$$\frac{M ( t )}{{M}_{0}} ≅ exp ( - 5.76 {D}_{a e} t / {R}_{f}^{2} ) \qquad ( 50 b )$$

From Eq. (50b), a rough estimate of the time constant for removing the absorptive excipient from the expanded fiber may be written as:

$${τ}_{f} ≅ \frac{{R}_{f}^{2}}{5.76 {D}_{a e}} \qquad ( 51 )$$

In the non-limiting experiment, the time constant, τ_{f}˜R_{f}²/5.76D_{HPMC}, was greater than about 8 hours. Thus, using R_{f}˜80 μm, the diffusivity, Dₐₑ˜R_{f}²/5.76τ_{f}, was smaller than about 4×10⁻¹⁴ m²/s.

By contrast, the self-diffusivity of an absorptive excipient, D_{self}, in water or a physiological fluid may be estimated by an adapted form of the Stokes-Einstein equation:

$${D}_{self} = \frac{{k}_{b} T}{6 π {r}_{e} μ} \qquad ( 52 )$$

where k_{b} is Boltzmann's constant, T the temperature of the fluid, rₑ is the radius of the excipient molecule, and μ the viscosity of water or the physiological fluid. The radius of an excipient molecule may be approximated as:

$${r}_{e} ≅ {( \frac{3 {M}_{w , e}}{4 π {N}_{A} {ρ}_{e}} )}^{1 / 3} \qquad ( 53 )$$

where M_{w,e} is the molecular weight of the excipient, N_{A} is Avogadro's number, and ρₑ the density of the excipient. For the non-limiting parameters of HPMC 120 k in water, M_{w,e}=120 kg/mol, ρₑ=1300 kg/m³, T=310 K, and μ=0.001 Pa·s, k_{b}=1.38×10⁻²³ m²kg/s²K, N_{A}=6.022×10²³/mol, the self-diffusivity, D_{self}≅6.7×10⁻¹¹ m²/s.

The results and calculations above suggests that the diffusivity of HPMC 120 k through the fiber was at least about 3 orders of magnitude smaller than the self-diffusivity of HPMC 120 k in water at 37 degree Celsius.

### EXPERIMENTAL EXAMPLES

### Part 2

The following examples present additional ways by which the disclosed dosage forms may be prepared and analyzed, and will enable one of skill in the art to more readily understand the principle of the invention herein. The examples are presented by way of illustration and are not meant to be limiting in any way.

### Example 2.1: Preparation of Fibrous Dosage Forms

First, particles of ibuprofen (a non-limiting model drug), Eudragit L100-55 (a strength-enhancing, enteric excipient), and barium sulfate (a gastrointestinal contrast agent) were mixed with liquid dimethylsolfoxide (DMSO) solvent to form a uniform suspension. Then the hydroxypropyl methylcellulose with a molecular weight of 120 kg/mol (HPMC 120 k) was mixed with the suspension. The masses of ibuprofen, Eudragit L100-55, barium sulfate, and HPMC 120 k per ml of DMSO in the formulation were 64, 64, 137, and 192 mg.

The mixture was extruded through a laboratory extruder to form a uniform viscous paste. The viscous paste was then put in a syringe equipped with a hypodermic needle of inner radius, Rₙ=84 μm. The paste was extruded through the needle to form a wet fiber that was patterned layer-by-layer as a fibrous dosage form with cross-ply structure. The nominal fiber radius in the dosage forms, Rₙ, was 84 μm, and the nominal inter-fiber spacing, λₙ, in a layer was 450 μm.

After patterning, the solvent was evaporated to solidify the dosage forms. The dosage forms were first put in a vacuum chamber maintained at a pressure of 100 Pa and a temperature of 20° C. for a day. Then they were exposed to an airstream of 60° C. and velocity 1 m/s for 60 min at ambient pressure.

After solvent evaporation, the solid dosage forms consisted of 42% HPMC 120 k, 30% barium sulfate, 14% ibuprofen, and 14% Eudragit L100-55 by weight. They were trimmed to 5 mm thick circular disks with nominal diameter 13-14 mm.

Two types of dosage form were produced. The first dosage form was coated with a hydrophilic sugar coating. The coating solution consisted of ethanol saturated with sucrose; it was held at −20° C. The dosage form was dipped into the coating solution and exposed to a pressure of 200 Pa right after for about an hour to evaporate the ethanol. The dipping-evaporation process was repeated three times. Because the hydrophilic sugar coating dissolves rapidly upon contact with water, this dosage form is referred to in the non-limiting experimental examples herein as “uncoated”.

The second dosage form was coated with an enteric coating. Two coating solutions were used: (I) 1.33 mg Eudragit L100-55 in 40 ml acetone, and (II) 2 ml Kollicoat SR in 20 ml deionized water. Both coating solutions were held at room temperature. The dosage form was dipped into the coating solution and exposed to a pressure of 200 Pa right after for about an hour to evaporate the solvent. The dipping-evaporation process was repeated 6 times for solution I, and 3 times for solution II. Because the enteric coating does not dissolve in acidic water, this dosage form is referred to in the non-limiting experimental examples herein as “coated”.

### Example 2.2 Scanning Electron Micrographs

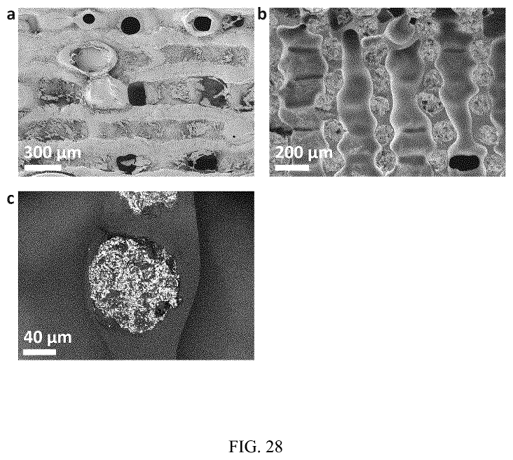

The microstructures of the fibrous dosage forms dip-coated with enteric excipient were imaged by a Zeiss Merlin High Resolution SEM with a GEMINI column. The top surfaces were imaged after coating the sample with a 10-nm thick layer of gold. The cross-sections were imaged after the sample was cut with a thin blade (MX35 Ultra, Thermo Scientific, Waltham, Mass.) and coated with gold as above. The specimens were imaged with either an in-lens secondary electron or a backscattered electron detector, at an accelerating voltage of 5 kV, and a probe current of 95 pA.The microstructures of the dosage forms dip-coated with enteric excipient are shown in FIGS. 28a-28c. FIG. 28a illustrates the top view of the dosage form. The top layer was mostly covered by the coating, but voids of about 100-300 μm in diameter were also present.

FIGS. 28b and 28c show the cross-sectional images. The fibers in the interior were coated; the coating bridged the neighboring fibers vertically, but not horizontally. Thus, the microstructure of the enteric-excipient-coated dosage forms comprised vertical walls of thickness, 2R₀, and vertical square channels of width, λ₀-2R₀. From FIGS. 28b and 28c, the fiber radius, R₀, was about 55 μm, and the inter-fiber spacing, λ₀, was 294 μm, Table 3.

TABLE 3
Microstructural parameters of the fibrous dosage forms.

| | R₀ (μm) | λ₀ (μm) | φₛ | φ_{f} | φ_{ec} |
|---|---|---|---|---|---|
| Enteric coated | 55 | 294 | 0.61 | 0.40 | 0.21 |

R₀: fiber radius; λ₀: inter-fiber distance; φₛ: volume fraction of solid; φ_{f}: volume fraction of fibers; φ_{ec}: volume fraction of enteric coating.
R₀ and λ₀ were obtained from FIG. 28.
The volume fractions were obtained from Eqs. (54)-(58).
The nominal process parameters were: Rₙ = 84 μm, λₙ = 450 μm.
Moreover, the half-thickness of the dosage form, H₀ = 2.5 mm, and the number of patterned layers, nₗ = 60.

Several microstructural parameters can be derived for this microstructure. The volume fraction of voids may be expressed as:

$${φ}_{v} = \frac{{( {λ}_{0} - 2 {R}_{0} )}^{2}}{{λ}_{0}^{2}} \qquad ( 54 )$$

The volume fraction of the solid walls (fiber and coating) may be written as:

$${φ}_{s} = 1 - {φ}_{v} = 1 - \frac{{( {λ}_{0} - 2 {R}_{0} )}^{2}}{{λ}_{0}^{2}} = \frac{4 {R}_{0}}{{λ}_{0}} - \frac{4 {R}_{0}^{2}}{{λ}_{0}^{2}} \qquad ( 55 )$$

The volume fraction of fibers (without the coating) may be expressed as:

$${φ}_{f} = ξ \frac{π {R}_{0}}{2 {λ}_{0}} \qquad ( 56 )$$

where ξ is the ratio of the “nominal” thickness of the dosage form (point contacts between fibers) and the “real” thickness of the dosage form (flattened fiber-to-fiber contacts):

$$ξ = \frac{2 {R}_{0} {n}_{l}}{2 {H}_{0}} = \frac{{R}_{0} {n}_{l}}{{H}_{0}} \qquad ( 57 )$$

Here nₗ is the number of stacked layers and H₀ the half-thickness of the solid dosage form.

The volume fraction of the enteric coating may be written as:

$${φ}_{e c} = {φ}_{s} - {φ}_{f} = \frac{4 {R}_{0}}{{λ}_{0}} - \frac{4 {R}_{0}^{2}}{{λ}_{0}^{2}} - ξ \frac{π {R}_{0}}{2 {λ}_{0}} \qquad ( 58 )$$

As listed in Table 3, for the relevant parameters of the dosage forms with enteric-excipient-coated fibers, φₛ=0.61, φ_{f}=0.4, and φ_{ec}=0.21.

The microstructures of the dosage forms dip-coated with sugar were similar to those with enteric-excipient coating. Because the sugar coating only serves the purpose to minimize the percolation time into the dosage form, and dissolves upon contact with water or gastric fluid, its volume fraction is not further characterized.

### Example 2.3 Expansion of the Dosage Forms Due to Water Diffusion

The dosage forms were immersed in a beaker filled with 800 ml of the dissolution fluid (0.1 M hydrochloric acid (HCl) in deionized (DI) water at 37° C.). The fluid was stirred with a paddle rotating at 50 rpm. The samples were imaged at different times by a Nikon DX camera. Expansion was monitored by imaging the samples at regular time intervals with a Nikon DX digital camera.

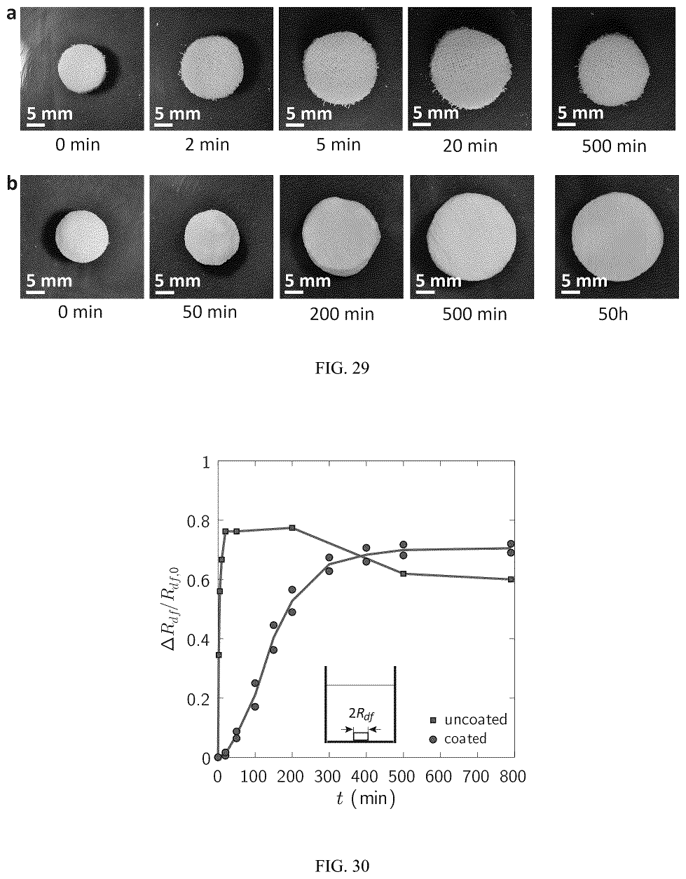

Images of the dosage forms at various times after immersion in the dissolution fluid are shown in FIG. 29. The normalized radial expansion of the dosage form, ΔR_{df}/R_{df,0}, is plotted versus time in FIG. 30.

The uncoated dosage form rapidly expanded and transformed into a semi-solid or highly viscous mass, FIG. 29a. The normalized expansion was 0.56 by 5 min and 0.76 by 20 min. The semi-solid or viscous mass was stabilized for over 10 hours, albeit the normalized expansion slightly decreased, from 0.77 at 200 minutes to 0.6 at 800 minutes, FIGS. 29a and 30.

The enteric coated dosage form expanded slower; ΔR_{df}/R_{df,0} was about 0.08 at 50 minutes, and then it increased gradually to 0.53 by 200 minutes, and plateaued to 0.7 by 500 min, FIGS. 29b and 30. The dimensions of the expanded dosage form then were essentially unchanged for more than a day.

### Example 2.4 Diametral Compression Tests

To determine the mechanical properties of the expanded, semi-solid masses (or dosage forms), the dosage forms were first soaked in a dissolution fluid (0.1 M HCl in deionized water at 37° C.) until they did not expand any further. The uncoated dosage forms were soaked for 30 mins, and the enteric-excipient-coated forms for 6 hours.

Diametral compression tests were then conducted using a Zwick Roell mechanical testing machine equipped with a 10 kN load cell and compression platens. The relative velocity of the platens was 2 mm/s. The test was stopped as soon as the specimen fractured visibly.

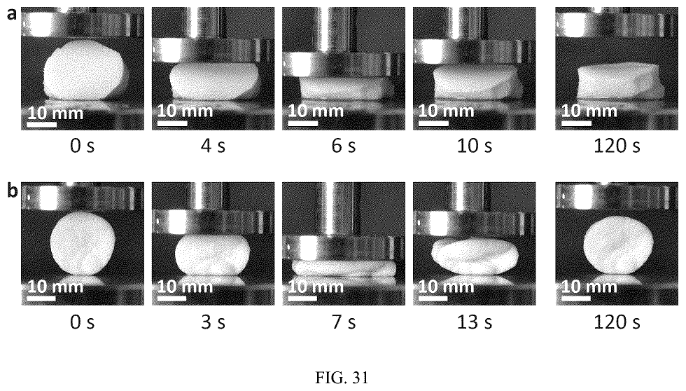

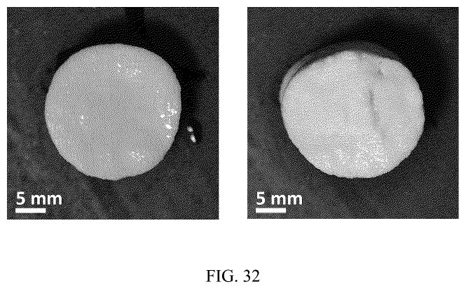

FIG. 31 presents images of diametral compression of the expanded semi-solid masses or dosage forms. The expanded, uncoated dosage form barely supported its own weight, FIG. 31a. Upon compression the dosage form deformed further and fractured. As the load was released, the dosage form did not regain its original shape.

The expanded, coated dosage form, by contrast, was much stiffer, FIG. 31b. Upon compression, the dosage form deformed, and as the load was released it sprang back and regained a shape and size similar to that of the original form. Nonetheless, the dosage form exhibited a crack along the axis of symmetry after compression, as shown in FIG. 32.

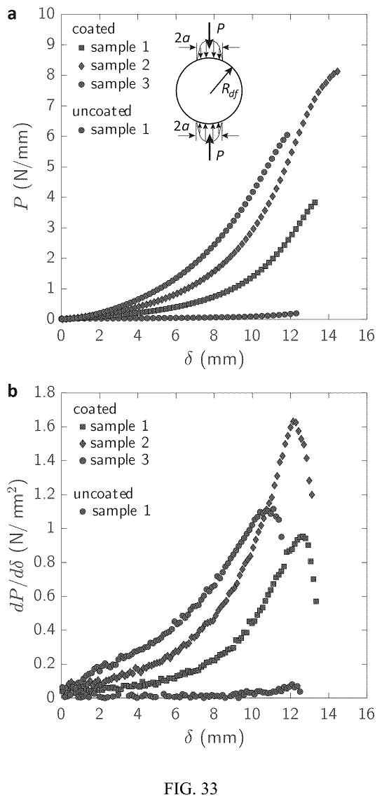

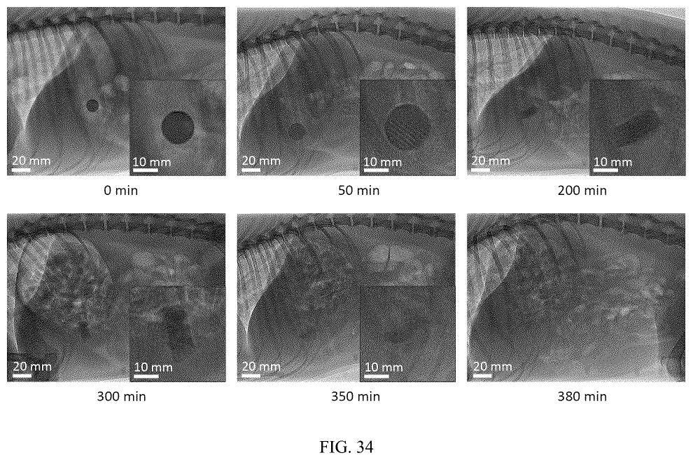

FIG. 33a presents the results of the load per unit length, P, versus displacement, δ, during diametral compression of the two types of dosage form. The slopes, dP/dδ, are plotted in FIG. 33b. For all dosage forms, up to a displacement of about 10-13 mm the load and its slope increased with displacement. But after that the P-δ curve exhibited an inflection point and the slope decreased. At a given displacement, both P and dP/dδ of the dosage forms with enteric-excipient-coated fibers were about 20-30 times those of the dosage forms with sugar-coated fibers.

For data analysis, the expanded dosage form is considered a linear elastic cylinder of radius, R_{df}, subjected to diametral compression by two hard, flat platens as shown in the inset of FIG. 33a. From the equations of elasticity, for small displacements the relative displacement of the platens may be approximated by (for further details, see, e.g., A. H. Blaesi, N. Saka, Int. J. Pharm. 509 (2016) 444-453; or K. L. Johnson, “Contact Mechanics”, Cambridge University Press, Cambridge, UK, 1985):

$$δ = 2 P \frac{1 - {v}^{2}}{π {E}_{df}} ( 2 ln \sqrt{\frac{4 π {R}_{df} {E}_{df}}{P}} - 1 ) \qquad ( 59 )$$

where P is the force per unit length along the cylinder surface (e.g., the load per unit length along the thickness of the expanded dosage form), v the Poisson's ratio, and E_{df} the elastic modulus of the expanded dosage form.

By inserting the experimental P and δ values from FIG. 33a in Eq. (59), and using v≈0.5, the elastic modulus of the dosage form can be estimated. For the expanded, uncoated dosage form, E_{df}=0.0075 MPa (7.5 kPa or ˜10⁻⁵ GPa), Table 4. This elastic modulus is of the order of that of gelatin (e.g., as in Jello); it is so low that the dosage form may also be considered a viscous gel or a viscous mass rather than an elastic solid or semi-solid (for further details related to materials classification, see, e.g., M. F. Ashby, Materials selection in mechanical design, Third ed., Butterworth-Heinemann, Oxford, UK, 2005). For the expanded, coated dosage form, E_{df}=0.098 MPa (˜10⁻⁴ GPa). This value is comparable to the modulus of low-stiffness, highly flexible polymer foams, such as foams of natural rubber or silicone (for further details related to materials classification, see, e.g., M. F. Ashby, Materials selection in mechanical design, Third ed., Butterworth-Heinemann, Oxford, UK, 2005).

Excessive plastic deformation, or fracture, of the dosage form may be observed if Eq. (59) is severely violated, i.e., if dP/dδ is at a maximum or P is at an inflection point. From FIG. 33 the inflection points, or loads at fracture, P_{f,df}=0.18 N/mm for the uncoated dosage form, and P_{f,df}=4.66 N/mm for the coated dosage forms, Table 4.

From the load at fracture the tensile strength of the dosage form may be estimated:

$${σ}_{f , df} ≅ \frac{{P}_{f , df}}{π {R}_{df}} \qquad ( 60 )$$

As listed in Table 4, for the expanded, uncoated dosage form, σ_{f,df}≈0.005 MPa, and for the coated, σ_{f,df}≈0.135 MPa. Again, the tensile or fracture strength of the expanded uncoated dosage form was so low that the dosage form may also be considered a viscous gel rather than an elastic solid. The tensile or fracture strength of the expanded coated form was more than an order of magnitude greater than that of the uncoated, and comparable to that of low-stiffness, highly flexible polymer foams (for further details related to materials classification, see, e.g., M. F. Ashby, Materials selection in mechanical design, Third ed., Butterworth-Heinemann, Oxford, UK, 2005, and references therein).

Thus, the stiffness and strength of the expanded dosage forms was substantially increased by the enteric coating (e.g., by coating the fibers with strength-enhancing excipient). In other words, the stiffness and strength of the expanded dosage forms increase greatly by increasing the weight fraction of strength-enhancing excipient in the dosage form, or by increasing the density of strength-enhancing excipient in the dosage form (e.g., by increasing the mass of strength-enhancing excipient in the dosage form per unit volume of the dosage form).

Moreover, it should be noted that both the expanded uncoated and the expanded coated dosage forms were soft materials that are unlikely to injure the gastrointestinal mucosa.

TABLE 4
Mechanical properties of expanded fibrous dosage forms.

| | E_{df} (MPa) | P_{f,df} (N/mm) | σ_{f,df} (MPa) |
|---|---|---|---|
| Uncoated dosage form | | | |
| Sample 1 | 0.0075 | 0.18 | 0.005 |
| Coated dosage forms | | | |
| Sample 1 | 0.076 | 3.31 | 0.096 |
| Sample 2 | 0.117 | 5.61 | 0.162 |
| Sample 3 | 0.102 | 5.05 | 0.146 |
| Average | 0.098 | 4.66 | 0.135 |
| Std | 0.017 | 0.98 | 0.028 |

E_{df}: elastic modulus of expanded dosage form; P_{f,df}: load per unit length at fracture; σ_{f,df}: stress at fracture
The properties were obtained from the diametral compression tests reported in FIG. 33, and Eqs. (59) and (60).
The properties of the acidic water-soaked enteric coating, E = 5.7 MPa and σ_{f} = 1.8 MPa (Table 6).

### Example 2.5 Gastric Residence Time of the Dosage Forms in Dogs

Two healthy beagle dogs (13-15 kg; three-year old; female; not castrated) were assigned five experiments comprising either coated and uncoated dosage forms. The dogs fasted for 18 hours prior to the experiment.

All dosage forms were administered to an awake dog, together with 30 ml water. The position of the dosage form was monitored by fluoroscopic imaging at the time points shown in FIGS. 34-36 (using a Philips Allura Clarity biplanar fluoroscopy system). Between imaging the dogs were allowed to roam about freely.

At 4-6 hours and at 30 hours after ingestion, 180 grams of basic dry food (Sensinesse 25/13, Petzeba AG, Alberswil, Switzerland) was given. No sedatives, anesthesia, or other supplements were administered before, during, or after the experiment.

The study was designed aiming to Replace animal experiments with non-sentient alternatives, Reduce animal experiments to minimize the number of animals used, and Refine animal experiments so that they cause minimum pain and distress. All procedures were conducted in compliance with the Swiss animal welfare act, and were approved by governmental authorities.

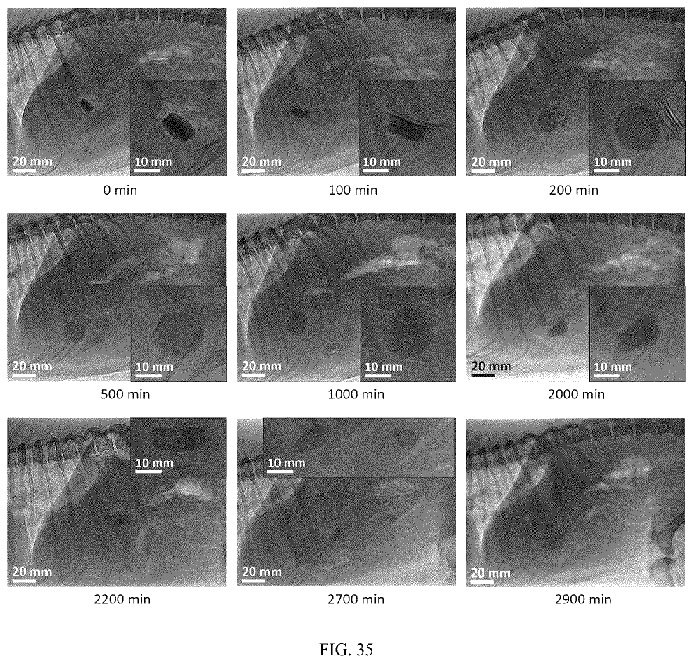

FIGS. 34 and 35 present fluoroscopic images of the dosage forms at various times after administration to a dog.

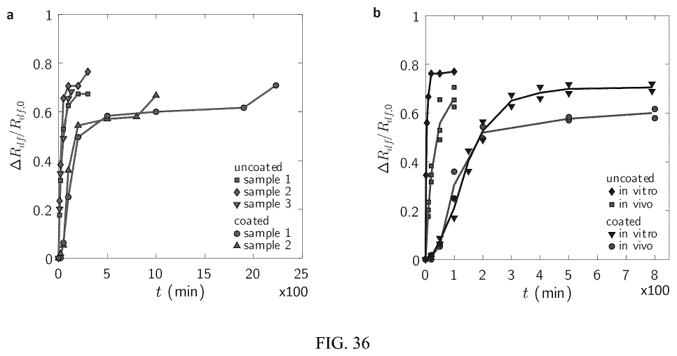

As shown in FIG. 34, the uncoated dosage form passed from the mouth into the stomach in less than a minute. In the stomach it expanded to a normalized radial expansion, ΔR_{df}/R_{fd,0}=0.63 by 100 minutes, and then plateaued to ΔR_{df}/R_{df,0}=0.67, FIG. 36a and Table 5. Thus the in vivo expansion rate was about a tenth of that measured in vitro, FIG. 36b. After about 300 minutes, as food was given to the dog, the dosage form showed visible cracks. The cracks grew rapidly and resulted in fracture at about 350 minutes. The fragments then passed into the intestines where they dissolved. By about 380 minutes (6.3 hours) the entire dosage form was essentially dissolved.

As shown in FIG. 35, the coated dosage form, too, passed from the mouth into the stomach in less than a minute. Similar to the in vitro results, it then expanded at a moderate rate to a normalized radial expansion, ΔR_{df}/R_{df,0}=0.5 by 200 minutes, and 0.6 by 500 minutes (FIG. 36b and Table 5). The integrity of the dosage form was mostly preserved until 2200-2700 minutes (37-45 hours) after ingestion. At 2700 minutes, fragments were seen in the intestine. The fragments dissolved rapidly; by 2900 minutes (48 hours), they were essentially invisible.

TABLE 5
Properties of fibrous dosage forms in vivo.

| | tₑₓₚ (min) | ΔR_{df}/R_{df,0} | δₘₐₓ (mm) | tᵣₑₛ (h) |
|---|---|---|---|---|
| Uncoated | | | | |
| Sample 1 | 100 | 0.67 | 10 | 6.3 |
| Sample 2 | 50 | 0.11 | 10 | 5.5 |
| Sample 3 | 100 | 0.68 | 11 | 2.5 |
| Average | 83 | 0.71 | 10.3 | 4.8 |
| Coated | | | | |
| Sample 1 | 200 | 0.60 | 6.5 | 41 |
| Sample 2 | 200 | 0.58 | 6.5 | 20 |
| Average | 200 | 0.59 | 6.5 | 30.5 |

tₑₓₚ: time to expand dosage form to greater than 90% of the terminal value; ΔR_{df}/R_{df,0}: terminal nominal expansion; δₘₐₓ: maximum deformation due to contracting stomach walls; tᵣₑₛ: gastric residence time
The data were derived from FIGS. 34-37.

Thus, unlike in vitro, in vivo the dosage forms fragmented and dissolved eventually. Fragmentation was due to contraction pulses by the stomach walls that occurred about every 10-30 seconds.

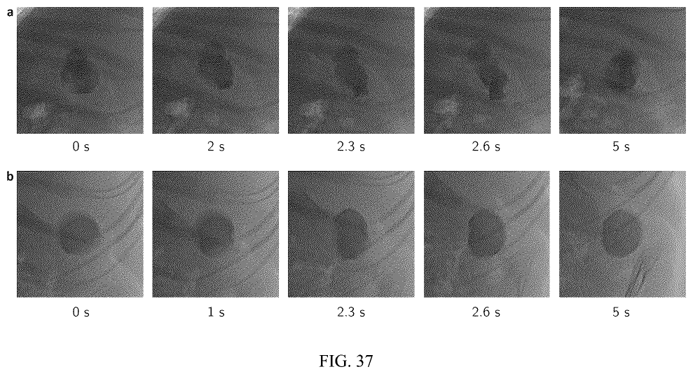

FIG. 37a shows a fluoroscopic image sequence of an uncoated dosage form during a contraction pulse by the stomach walls at about 2 hours after ingestion. The (expanded) dosage form was circular and of diameter 23 mm initially. At 2.6 s, the dosage form was squeezed by about 11 mm to a width of roughly 12 mm. At 5 s the dosage form regained a round shape of roughly the initial diameter. Soon after the images were taken, however, the dosage form fractured.

FIG. 37b shows a fluoroscopic image sequence of a coated dosage form during a contraction pulse at about 7 hours after ingestion. Initially, the (expanded) dosage form was circular and of diameter 23 mm. At 1 s, the dosage form was diametrically pinched, and at 2.3 s it was diametrically compressed by about 6.5 mm to a width of about 16.5 mm. The dosage form regained its original shape after about 5 s. The compression-spring back cycles were repeated for several more hours as the coated dosage form was retained in the stomach.

For an analysis of the forces applied on the dosage form and the gastric residence, we may consider the non-limiting force field shown in FIG. 12 comprising diametrically opposed cyclic loads per unit length, P, with maximum load per unit length, Pₘₐₓ, acting on the expanded semi-solid or viscous dosage form. From the results, the maximum compression, δₘₐₓ, was about 6.5 mm in vivo, FIG. 37b and Table 5. In the in vitro experiments, at δ=6.5 mm P was about 1 N/mm, FIG. 33a. Thus, in the in vivo experiments the maximum cyclic load intensity imposed by the stomach walls, Pₘₐₓ˜1 N/mm.

The corresponding cyclic stress (tension) along the axis of symmetry may be approximated as:

$${σ}_{max} = \frac{{P}_{max}}{π {R}_{df}} \qquad ( 61 )$$

where R_{df} is the radius of the expanded dosage form. For Pₘₐₓ=1 N/mm and R_{df}=11.5 mm, by Eq. (61) σₘₐₓ=0.028 MPa. This is one-fifth of the fracture strength, σ_{f,df}=0.135 MPa, obtained from the monotonic, in vitro diametral compression test, Table 4. Thus, in vivo the dosage form may have exhibited fatigue fracture.

By Eq. (31), if the dosage form disintegrates due to fatigue fracture, the gastric residence time may be estimated as:

$${t}_{r} ∼ {{t}_{pulse} ( \frac{{P}_{max}}{π {R}_{df} {σ}_{f , se} {C}_{8} {φ}_{se}^{3 / 2}} )}^{1 / b} \qquad ( 62 )$$

where σ_{f,se} is the fracture strength of the strength-enhancing excipient, σₛₑ the volume fraction of the strength-enhancing excipient in the dosage form (e.g., the volume fraction of the enteric coating), and C₈ a constant, typically about 0.65.

Thus, for tₚᵤₗₛₑ=20 s, Pₘₐₓ=1 N/mm, R_{df}=11.5 mm, σ_{f,se}=1.8 N/mm², C₈=0.65, and φₛₑ≈φ_{c}=0.21, by Eq. (62) the gastric residence time, tᵣ˜31 hours if the constant, b˜−0.162.

### Example 2.6 Solubility and Sorption of Deionized Water with 0.1 M HCl in Strength-Enhancing Excipient

Strength-enhancing excipient (Methacrylic acid-ethyl acrylate copolymer (1:1), with a molecular weight of about 250 kg/mol, also referred to herein as “Eudragit L100-55”) was received from Evonik, Essen, Germany.

Solid films of the strength-enhancing excipient were prepared by dissolution of Eudragit L100-55 in DMSO to form a viscous solution, pouring the solution in a metal dish to form a film, and evaporating DMSO in a vacuum chamber at a pressure of about 1 mbar and a temperature of about 50° C. for about a day. The thickness of the solid, frozen films, h₀, was about 250 μm.

For determining the properties of the solid films, the solid films were first immersed in a relevant dissolution fluid (water with 0.1 M HCl at 37° C.). The weight of the film was then measured at specific time points with a Mettler Toledo analytical balance. The weight fraction of water (or dissolution fluid) in the film, w_{w}, was determined by:

$${w}_{w} = \frac{m ( t ) - {m}_{0}}{m ( t )} \qquad ( 63 )$$

where m(t) is the mass of the water-soaked film at time t after immersion in the dissolution fluid, and m₀ is the mass of the solid film initially.

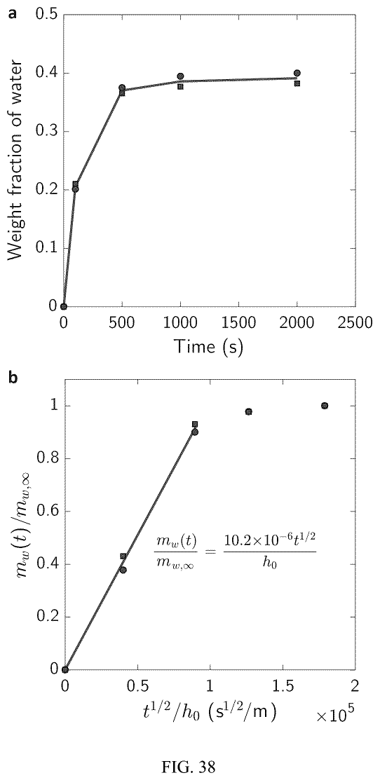

FIG. 38a is a plot of the weight fraction of water (or dissolution fluid) in the films versus time after immersion. The weight fraction of water increased with time at a degressive rate, and plateaued out at about 2000 seconds to a value of about 0.39. Thus, the “solubility” of the dissolution fluid in the strength-enhancing Eudragit L100-55 excipient film was about 39 weight percent, or roughly 390 mg/ml.

FIG. 38b is a plot of the mass of dissolution fluid absorbed by the film at time t, m_{w}(t)=m(t)−m₀, divided by the mass absorbed at “infinite” time (e.g., at 2000 seconds), m_{w,∞}=m(t=2000 s)−m₀, versus t^{1/2}/h₀. For small times, the fit of the data was linear; thus, initially the data followed a curve of the form

$$\frac{{m}_{w} ( t )}{{m}_{w , ∞}} = {{k}_{s} ( \frac{t}{{h}_{0}^{2}} )}^{1 / 2} \qquad ( 64 )$$

where kₛ is a sorption constant. From FIG. 38b, the sorption constant, kₛ=10.2×10⁻⁶ m/s^{1/2}.

According to Crank, in Fickian diffusion, for small times the mass of water sorbed at time t, m_{w}(t) divided by the mass of water (or physiological fluid) sorbed at “infinite” time, m_{w,∞}, by a plane film may be approximated by:

$$\frac{{m}_{w} ( t )}{{m}_{w , ∞}} ≅ \frac{4}{\sqrt{π}} {( \frac{{D}_{w} t}{{h}_{0}^{2}} )}^{1 / 2} \qquad ( 65 )$$

where D_{w} is the diffusivity of water (or physiological fluid) in the film.

Thus, D_{w}, may be estimated from the data plotted in FIG. 38b as:

$${D}_{w} ≅ \frac{π {k}_{s}^{2}}{16} \qquad ( 66 )$$

For kₛ˜10.2×10⁻⁶ m/s^{1/2}, D_{w}˜2.04×10⁻¹¹ m²/s.

### Example 2.7 Mechanical Properties of Acidic Water-Penetrated Eudragit L100-55 Films

Solid films of Eudragit L100-55 were prepared by dissolving 3 g Eudragit powder in 40 ml Acetone, pouring the solution in a polyethylene box with dimensions about 100 mm×60 mm to form a film, and drying at room temperature for about a day. The solid, frozen films were then punched into tensile specimen according to DIN 53504, type S 3A. The specimen thickness was 150-250 μm.

The tensile specimens were soaked in a dissolution fluid (water with 0.1 M HCl at 37° C.) for about an hour. Subsequently, the water-soaked specimen were loaded in a Zwick Roell Mechanical Testing machine equipped with a 20-N load cell. The initial distance between grips was 28 mm. During tensile testing the grips receded at a relative velocity of 2 mm/s, and the force and distance between grips were recorded. The test was stopped when the sample ruptured, and the load decreased to less than 80% of the maximum load.

From the recordings of force and distance and the geometry of the tensile specimen, the nominal stress, σ, and strain, ε, in the specimen can be derived as:

$$σ = \frac{F}{W h} \qquad ( 67 )$$

$$ε = \frac{Δ L}{{L}_{0}} \qquad ( 68 )$$

where F is the tensile force applied on the film, W the width of the narrow section of the water-soaked tensile specimen, h its thickness, ΔL the distance travelled by the grips, and L₀ the initial distance.

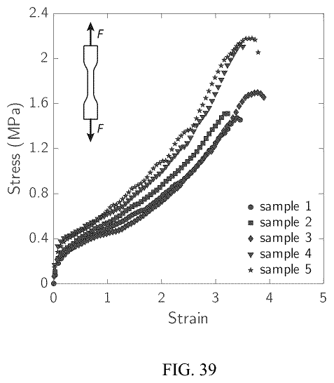

FIG. 39 plots the nominal stress, σ, versus engineering strain, ε, of the acidic water-soaked tensile specimen films comprising strength-enhancing Eudragit L100-55 excipient. Initially, the stress increased steeply and roughly linearly with strain. At a strain of about 0.06-0.12, the slope decreased substantially. The stress then increased with strain at a non-linear, progressive rate. Eventually, when the sample ruptured, the stress dropped abruptly.

From the stress-strain curves several properties of the acidic water-soaked films can be derived. The elastic modulus,

$$E = \frac{Δ σ}{Δ ε} {\vert}_{σ < {σ}_{y}} \qquad ( 69 )$$

where the yield strength, σ_{y}, is defined here as the first stress on the curve at which an increase in strain occurs without an increase in stress. The fracture strength, σ_{f}, (also referred to herein as “tensile strength”) is the maximum stress on the curve.

As listed in Table 6, the average values of the measured properties, E=5.7 MPa (5×10⁻³ GPa), σ_{y}=0.26 MPa, and σ_{f}=1.8 MPa. These values are comparable to the properties of typical low-strength elastomers or rubbers (for further details related to materials classification, see, e.g., M. F. Ashby, Materials selection in mechanical design, Third ed., Butterworth-Heinemann, Oxford, UK, 2005).

TABLE 6
Properties of acidic water-soaked Eudragit L100-55 films
derived from tension tests.

| | E (MPa) | σ_{y} (MPa) | ε_{y} | σ_{f} (MPa) | ε_{f} |
|---|---|---|---|---|---|
| Sample 1 | 3.6 | 0.19 | 0.10 | 1.48 | 3.43 |
| Sample 2 | 4.9 | 0.26 | 0.12 | 1.52 | 3.21 |
| Sample 3 | 5.7 | 0.21 | 0.06 | 1.70 | 3.76 |
| Sample 4 | 7.3 | 0.34 | 0.08 | 2.14 | 3.47 |
| Sample 5 | 6.9 | 0.30 | 0.09 | 2.18 | 3.64 |
| Average | 5.7 | 0.26 | 0.09 | 1.80 | 3.50 |
| Std | 1.35 | 0.06 | 0.02 | 0.3 | 0.19 |

E: elastic modulus; σ_{y}: yield strength; ε_{y}: strain at yield; σ_{f}: of stress at fracture; ε_{f}: strain at fracture.
The properties were obtained from tensile tests reported in FIG. 39.
The elastic modulus, E, was derived from Eq. (69).
σ_{y} was defined as the first stress at which an increase in strain occured without an increase in stress.
σ_{f} the maximum stress.

### APPLICATION EXAMPLES

In some embodiments, the amount of active ingredient contained in a dosage form disclosed in this invention is appropriate for administration in a therapeutic regimen that shows a statistically significant probability of achieving a predetermined therapeutic effect when administered to a relevant population. By way of example but not by way of limitation, active ingredients may be selected from the group consisting of acetaminophen, aspirin, caffeine, ibuprofen, an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antihyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, or a vasodilator.

Moreover, while useful for improving almost any drug therapy, the disclosed dosage forms can be particularly beneficial for therapies that require tight control of the concentration in blood of drugs that are soluble or fairly soluble in acidic but sparingly soluble or practically insoluble in basic solution.

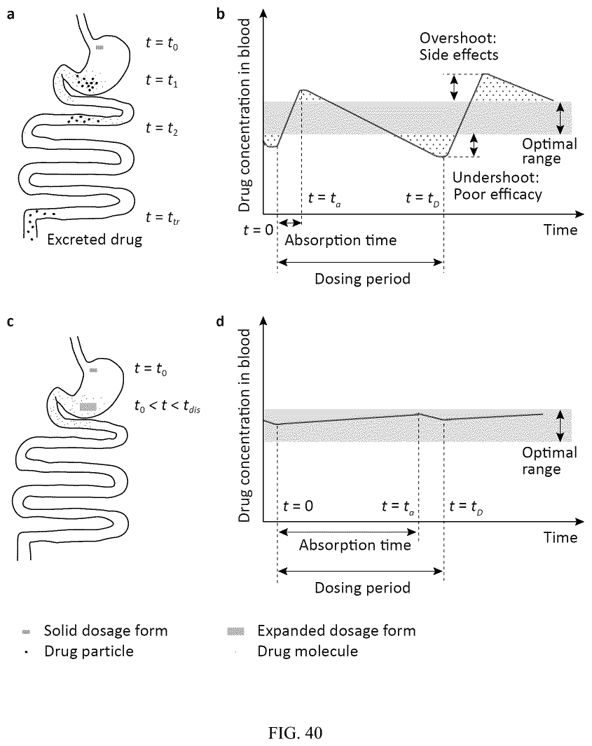

More specifically, as shown schematically in the non-limiting FIG. 40a, upon ingestion of a traditional, granular dosage form comprising drug that is soluble in acidic but insoluble in basic solution, the dosage form may fragment into its constituent particulates in the stomach, and release drug as particles and molecules. As the drug molecules may pass into the upper, acidic part of the intestines, they may enter the blood stream, and the drug concentration in blood may increase steeply, as shown in the non-limiting FIG. 40b. As the drug molecules may enter the lower, basic part, however, they may precipitate back as particles, which may not (or only very slowly) be absorbed, and the drug concentration in blood may decrease with time, FIG. 40b. Moreover, because drug may be excreted the bioavailability, understood herein as the mass of drug absorbed by the blood after ingestion of a dosage form divided by the mass of drug in the dosage form initially, may be low. The bioavailability may further be variable, because the transit times through and physico-chemical environment in the stomach and upper intestines can be variable. Consequently, the drug concentration in blood may fluctuate beyond the optimal range, and the efficacy and safety of the drug therapy may be compromised, FIG. 40b.

The disclosed dosage forms, by contrast, enable retention of the dosage form in the stomach and slower drug delivery rate over a prolonged time. For example, the disclosed dosage forms may be smaller than the diameter of the oesophagus (˜15-20 mm) to facilitate ingestion, as shown in the non-limiting FIG. 40c. But in the stomach they may expand rapidly to a size greater than the diameter of the pylorus (˜13-20 mm). Moreover, as the dosage forms expand they may transition from solid to semi-solid or highly viscous, and remain in a semi-solid or highly viscous state for prolonged time, thus precluding their immediate passage into the small intestine and eliminating any risk of mechanically injuring the gastric mucosa. Drug molecules may be released into the stomach slowly and over prolonged time, and be predominantly absorbed in the upper part of the gastrointestinal tract after their release. As a result, the drug absorption rate may be fairly steady over prolonged time, the bioavailability may be high, and the variability of the bioavailability may be low. Consequently, the drug concentration in blood may be well controlled within the optimal range, as shown in the non-limiting FIG. 40d; the efficacy of the drug therapy may be increased, and/or side effects of the therapy may be decreased.

In some embodiments, therefore, the dosage forms herein comprise one or more active ingredients or drugs that are more soluble in acidic solutions (e.g., in the stomach or duodenum) than in basic solutions (e.g., in the bowel or large intestine). Thus, in some embodiments, the dosage form comprises at least one active pharmaceutical ingredient having a pH-dependent solubility in a physiological or body fluid.

Furthermore, in some embodiments, the dosage form herein comprises at least one active pharmaceutical ingredient having a solubility that is at least five times greater in acidic solution than in basic solution. This includes, but is not limited to at least one active ingredient having a solubility that is at least 10 times, or at least 15 times, or at least 20 times, or at least 30 times, or at least 50 times greater in acidic solution than in basic solution. In the invention herein, a solution is understood “acidic” if the pH value of said solution is no greater than about 5.5. A solution is understood “basic” if the pH value of said solution is greater than about 5.5.

Moreover, in some embodiments, the dosage form herein comprises at least one active pharmaceutical ingredient that is a basic compound. In the invention herein, a compound is understood “basic” if the acid dissociation constant (e.g., the pKa value) of said compound is greater than about 5.5.

More generally, furthermore, the disclosed dosage forms can be beneficial for therapies that require tight or fairly tight control of the concentration in blood of drugs that are sparingly-soluble (e.g., poorly soluble) in an aqueous physiological fluid or gastro-intestinal fluid.

Thus, in some embodiments, the dosage form herein comprises at least one active pharmaceutical ingredient having a solubility no greater than 5 g/l in an aqueous physiological/body fluid under physiological conditions. This includes, but is not limited to at least one active ingredient having a solubility no greater than 2 g/l, or no greater than 1 g/l, or no greater than 0.5 g/l, or no greater than 0.2 g/l, or no greater than 0.1 g/l in an aqueous physiological or body fluid under physiological conditions.

It may be noted, moreover, that due to the greater bioavailability, with the disclosed dosage form the mass of drug a patient is recommended to or supposed to ingest to achieve a therapeutic effect may be lower than with the traditional dosage form.

Additionally, due to the capability of releasing drug into the upper gastrointestinal tract over prolonged time, the disclosed dosage form may enable to reduce the dosing frequency for treatment of a specific disease or medical condition. The “dosing frequency” is understood herein as the number of times a patient may ingest, or is recommended to ingest (e.g., by medical personnel such as a doctor, pharmacist, etc.), a drug dose in a given time. In other words, the “dosing frequency” may be understood as the reciprocal of the recommended time interval between two drug doses to be ingested by or administered to a patient. A “drug dose” may be understood as a specific drug mass to be ingested by or administered to a patient at a specific time. The specific drug mass may be included in one or more dosage forms.

The disclosed dosage form, therefore, can be beneficial for therapies comprising a drug with short half-life in blood or a human or animal body. The “half-life” is understood herein as the period of time required for a “maximum” concentration or “maximum” amount of drug in blood or in the body to be reduced by one-half, under the condition that no drug is delivered into the blood or body during said time period. The concentration of drug in blood may generally be estimated from measurements of the concentration of drug in blood plasma.

In some embodiments, accordingly, the dosage form herein comprises at least one active pharmaceutical ingredient having a half-life in a human or animal body (e.g., a physiological system) no greater than one day or 24 hours. This includes, but is not limited to a half-life in a human or animal body no greater than 22 hours, or no greater than 20 hours, or no greater than 18 hours, or no greater than 16 hours, or no greater than 14 hours, or no greater than 12 hours, or no greater than 10 hours, or no greater than 8 hours, or no greater than 6 hours, or no greater than 4 hours, or in the ranges 0.5-24 hours, 0.5-20 hours, 0.5-16 hours, 0.5-12 hours, 0.5-10 hours, 0.5-8 hours, or 0.5-6 hours.

Finally, the disclosed dosage forms can be manufactured by an economical process enabling more personalized medicine.

We claim:

1. A pharmaceutical dosage form comprising:
a drug-containing solid comprising an outer surface and an internal three dimensional structural framework of one or more structural elements, said framework contiguous with and terminating at said outer surface;
said elements having segments spaced apart from adjoining segments, thereby defining one or more free spaces in the drug-containing solid;
said elements further comprising at least one active ingredient and at least two excipients;
said at least two excipients comprising at least one physiological fluid-absorptive polymeric constituent and at least one strength-enhancing polymeric constituent;
wherein
upon exposure to a physiological fluid, said strength-enhancing excipient forms a fluid-permeable, semi-solid network mechanically supporting said framework; and
said fluid-absorptive excipient transitions to a viscous mass or a viscous solution expanding said framework along at least one dimension with absorption of said physiological fluid.

2. The dosage form of claim 1, wherein one or more phases comprising strength-enhancing excipient form a substantially continuous or connected structure along the lengths of one or more structural elements.

3. The dosage form of claim 1, wherein one or more free spaces are interconnected.

4. The dosage form of claim 3, wherein upon ingestion by a human or animal subject, physiological fluid percolates at least one interconnected free space and diffuses into one or more said elements, thereby expanding said framework in all dimensions and transitioning said framework to a semi-solid mass releasing said drug over time.

5. The dosage form of claim 1, wherein upon exposure to a physiological fluid, said framework expands to a length between 1.3 and 4 times its length prior to exposure to said physiological fluid.

6. The dosage form of claim 5, wherein upon prolonged exposure to a physiological fluid, said expanded framework or semi-solid mass maintains its length between 1.3 and 4 times the initial length for prolonged time.

7. The dosage form of claim 1, wherein upon exposure to a physiological fluid, said framework transitions to a semi-solid mass, and wherein said semi-solid mass comprises a substantially continuous or connected network of one or more strength-enhancing excipients.

8. The dosage form of claim 1, wherein the average thickness of the one or more structural elements is in the range of 1 μm to 1.5 mm.

9. The dosage form of claim 1, wherein the three dimensional structural framework comprises criss-crossed stacked layers of fibers.

10. The dosage form of claim 1, wherein the solubility of a physiological fluid in at least one absorptive excipient is greater than 600 mg/ml.

11. The dosage form of claim 1, wherein at least one absorptive excipient comprises hydroxypropyl methylcellulose.

12. The dosage form of claim 1, wherein at least one absorptive excipient is selected from the group comprising hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, or vinylpyrrolidone-vinyl acetate copolymer.

13. The dosage form of claim 1, wherein the solubility of a relevant physiological fluid in at least a strength-enhancing excipient is no greater than 750 mg/ml under physiological conditions.

14. The dosage form of claim 1, wherein at least a strength-enhancing excipient comprises an elastic modulus in the range of 0.5 MPa-100 MPa after soaking with a physiological fluid under physiological conditions.

15. The dosage form of claim 1, wherein at least a strength-enhancing excipient comprises a tensile strength in the range of 0.05 MPa-200 MPa after soaking with a physiological fluid under physiological conditions.

16. The dosage form of claim 1, wherein at least a strength-enhancing excipient comprises a strain at fracture greater than 0.5 after soaking with a physiological fluid under physiological conditions.

17. The dosage form of claim 1, wherein the volume or weight fraction of the one or more absorptive excipients in the three dimensional structural framework of one or more elements is in the range between 0.1 and 0.85.

18. The dosage form of claim 1, wherein the volume or weight fraction of the one or more strength-enhancing excipients in the three dimensional structural framework of one or more elements is in the range between 0.15 and 0.9.

19. The dosage form of claim 1, wherein at least one strength-enhancing excipient comprises an enteric polymer, said enteric polymer having a solubility at least 10 times greater in a basic solution having a pH value greater than 7 than in an acidic solution having a a pH value no greater than 5.

20. The dosage form of claim 1, wherein at least one strength-enhancing excipient comprises methacrylic acid-ethyl acrylate copolymer.

21. The dosage form of claim 1, wherein at least one strength-enhancing excipient is selected from the group comprising hydroxypropyl methyl cellulose acetate succinate, polyvinyl acetate, ethyl acrylate polymers (e.g., polymers including ethyl acrylate), methacrylate polymers (e.g., polymers including methacrylate), ethyl acrylate-methylmethacrylate copolymers, Poly[Ethyl acrylate, methyl methacrylate, trimethylammonioethyl methacrylate chloride], Poly[Ethyl acrylate, methyl methacrylate, trimethylammonioethyl methacrylate chloride], and ethylcellulose.

22. The dosage form of claim 1, wherein said at least two excipients form a solid solution through the thickness of one or more elements.

23. The dosage form of claim 1, wherein an element or framework comprises a plurality of segments having substantially the same weight fraction of physiological fluid-absorptive and/or strength-enhancing excipient distributed within the segments.

24. The dosage form of claim 1, wherein at least one free space is filled with matter removable by a physiological fluid under physiological conditions.

25. The dosage form of claim 1, wherein upon immersion in a physiological fluid the drug-containing solid transitions to a semi-solid mass, and wherein said semi-solid mass comprises an elastic modulus in the range of 0.005 MPa-15 MPa.

26. The dosage form of claim 1, wherein upon immersion in a physiological fluid the drug-containing solid transitions to a semi-solid mass, and wherein said semi-solid mass comprises a tensile strength in the range between 0.002 MPa and 15 MPa.

27. The dosage form of claim 1, wherein eighty percent of the drug content is released from the drug containing solid into a physiological fluid within 1 hour to 30 days after immersion of the drug-containing solid into said physiological fluid under physiological conditions.

28. The dosage form of claim 1, wherein upon ingestion by a human or animal subject, said dosage form is gastroretentive.

29. A pharmaceutical dosage form comprising:
a drug-containing solid comprising an outer surface and an internal three dimensional structural framework of one or more thin structural elements, said framework contiguous with and terminating at said outer surface;
said elements having segments spaced apart from adjoining segments, thereby defining one or more interconnected free spaces through the drug-containing solid;
said elements further comprising at least one active ingredient and at least two excipients;
said at least two excipients comprising at least one physiological fluid-absorptive polymeric constituent and at least one strength-enhancing polymeric constituent;
whereby
upon immersion in a physiological fluid, said fluid percolates at least one interconnected free space and diffuses into one or more said elements, so that the framework expands in at least one dimension and transitions to a semi-solid mass; wherein
said semi-solid mass releases drug over prolonged time.

30. A pharmaceutical dosage form comprising:
a drug-containing solid comprising an outer surface and an internal three dimensional structural framework of one or more structural elements, said framework contiguous with and terminating at said outer surface;
said elements having segments spaced apart from adjoining segments, thereby defining one or more interconnected free spaces through the drug-containing solid;
said elements further comprising at least one active ingredient and at least two excipients;
said at least two excipients comprising one or more fluid-absorptive polymeric constituents within which the solubility of a physiological fluid is greater than 600 mg/ml;
said at least two excipients further comprising one or more strength-enhancing polymeric constituents;
said one or more strength-enhancing polymeric constituents having an elastic modulus in the range between 0.2 MPa and 200 MPa, and a strain at fracture greater than 0.2 after soaking with a physiological fluid under physiological conditions;
wherein
upon exposure to a physiological fluid, said strength-enhancing excipient forms a fluid-permeable, semi-solid network mechanically supporting said framework; and
said fluid-absorptive excipient transitions to a viscous mass or a viscous solution expanding said framework along at least one dimension with absorption of said physiological fluid.
